(19) United States
(12) Patent Application Publication
(10) Pub. No.: US 2022/0348967 A1
(43) Pub. Date: Nov. 3, 2022

(54) Microorganisms With Improved Nitrogen Utilization For Ethanol Production

(71) Applicants: Novozymes A/S, Bagsvaerd (DK); Microbiogen Pty. Ltd., SYDNEY (AU)

(72) Inventors: Hamid Rismani Yazdi, Raleigh, NC (US); Sarah Schultheis Elliott, Wake Forest, NC (US); Paul Vincent Harris, Carnation, WA (US); Michael Glenn Catlett, West Sacramento, CA (US)

(73) Assignees: Novozymes A/S, Bagsvaerd (DK); Microbiogen Pty. Ltd., SYDNEY (AU)

(21) Appl. No.: 16/963,084

(22) PCT Filed: Jan. 29, 2019
(86) PCT No.: PCT/US2019/015682

§ 371 Date: Jul. 17, 2020

Related U.S. Application Data
(60) Provisional application No. 62/629,321, filed on Feb. 12, 2018.
(60) Provisional application No. 62/623,372, filed on Jan. 29, 2018.

(51) Int. Cl. C12P 7/06; C07K 14/39; C12N 15/52

(52) U.S. Cl. CPC C12P 7/06; C07K 14/39; C12N 15/52

(57) ABSTRACT

Described herein are fermention organisms, such as yeasts, comprising a genetic modification that increases or decreases expression of a transporter or regulator thereof, such as yeasts that express the FOT2 and FOTX transporters of SEQ ID NOs: 163 and 164. Also described are processes for producing a fermentation product, such as ethanol, from starch or cellulosic-containing material with the fermenting organisms.

### REFERENCE TO A SEQUENCE LISTING

This application contains a Sequence Listing in computer readable form, which is incorporated herein by reference.

### BACKGROUND

Production of ethanol from starch and cellulosic containing materials is well-known in the art.

The most commonly industrially used commercial process for starch-containing material, often referred to as a “conventional process”, includes liquefying gelatinized starch at high temperature (about 85° C.) using typically a bacterial alpha-amylase, followed by simultaneous saccharification and fermentation (SSF) carried out anaerobically in the presence of typically a glucoamylase and a Saccharomyces cerevisae yeast.

There are several processes in the art for saccharification of cellulose and hemicelluloses, and for fermentation of hydrolysates containing glucose, mannose, xylose and arabinose. Glucose and mannose are efficiently converted to ethanol during natural anaerobic metabolism. To obtain an economically relevant process at industrial scale, advances have been made to improve fermentation xylose within the hydrolysates.

Yeasts which are used for production of ethanol for use as fuel, such as in the corn ethanol industry, require several characteristics to ensure cost effective production of the ethanol. These characteristics include ethanol tolerance, low by-product yield, rapid fermentation, and the ability to limit the amount of residual sugars remaining in the ferment. Such characteristics have a marked effect on the viability of the industrial process.

Yeast of the genus Saccharomyces exhibits many of the characteristics required for production of ethanol. In particular, strains of Saccharomyces cerevisiae are widely used for the production of ethanol in the fuel ethanol industry. Strains of Saccharomyces cerevisiae that are widely used in the fuel ethanol industry have the ability to produce high yields of ethanol under fermentation conditions found in, for example, the fermentation of corn mash. An example of such a strain is the yeast used in commercially available ethanol yeast product called ETHANOL RED®.

The addition of exogenous protease to corn mash has been a strategic approach to increase availability amino nitrogen and accelerate rates of ethanol fermentation (See, e.g., Biomass 16 (1988) 2, pp. 77-87; U.S. Pat. No. 5,231,017; WO2003/066826; WO2007/145912; WO2010/008841; WO2014/037438; WO2015/078372). We also described expression of heterologous proteases in Saccharomyces cerevisiae for ethanol fermentation (WO2018/222990, the content of which is incorporated herein by reference).

Despite significant improvement of ethanol production processes over the past decade there is still a desire and need for providing improved processes of ethanol fermentation from starch and cellulosic containing material in an economically and commercially relevant scale.

### SUMMARY

Described herein are, inter alia, methods for producing a fermentation product, such as ethanol, from starch or cellulosic-containing material, and yeast suitable for use in such processes.

A first aspect relates to a yeast cells (e.g., recombinant yeast cells) comprising a genetic modification that increases or decreases expression of a transporter, or regulator thereof.

In one embodiment, the yeast cell is a Saccharomyces cerevisiae yeast cell comprising: (1) a heterologous polynucleotide encoding a transporter, and (2) a heterologous polynucleotide encoding a glucoamylase, alpha-amylase, or protease; wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164.

In one embodiment, the yeast cell comprises a heterologous polynucleotide encoding a transporter, wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164, and wherein the yeast cell comprises a recombinant genetic modification that increases expression of the transporter.

In one embodiment, the yeast cell comprises a heterologous polynucleotide encoding a transporter, wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164, and wherein the yeast further comprises a disruption to an endogenous transporter gene.

In one embodiment, the yeast cell is a Saccharomyces cerevisiae yeast cell comprising a heterologous polynucleotide encoding a transporter, wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164; and with the proviso that the yeast cell is other than: Saccharomyces cerevisiae MBG4851 (deposited under Accession No. V14/004037 at National Measurement Institute, Victoria, Australia) or a derivative thereof, Saccharomyces cerevisiae MBG4911 (deposited under Accession No. V15/001459 at National Measurement Institute, Victoria, Australia) or a derivative thereof, Saccharomyces cerevisiae MBG4913 (deposited under Accession No. V15/001460 at National Measurement Institute, Victoria, Australia) or a derivative thereof, Saccharomyces cerevisiae MBG4914 (deposited under Accession No. V15/001461 at National Measurement Institute, Victoria, Australia) or a derivative thereof, Saccharomyces cerevisiae MBG4930 (deposited under Accession No. V15/004035 at National Measurement Institute, Victoria, Australia) or a derivative thereof, Saccharomyces cerevisiae MBG4931 (deposited under Accession No. V15/004036 at National Measurement Institute, Victoria, Australia) or a derivative thereof, Saccharomyces cerevisiae MBG4932 (deposited under Accession No. V15/004037 at National Measurement Institute, Victoria, Australia) or a derivative thereof.

A second aspect relates to methods of producing a fermentation product from a starch-containing or cellulosic-containing material comprising: (a) saccharifying the starch-containing or cellulosic-containing material; and (b) fermenting the saccharified material of step (a) with the yeast cells of the first aspect.

In one embodiment, the method comprises liquefying the starch-containing material at a temperature above the initial gelatinization temperature in the presence of an alpha-amylase and a protease prior to saccharification. In one embodiment, the fermentation product is ethanol.

A third aspect relates to methods of producing a derivative of a yeast strain of the first aspect, comprising culturing a yeast strain of the first aspect with a second yeast strain under conditions which permit combining of DNA between the first and second yeast strains, and screening or selecting for a derivatived yeast strain comprising the heterologous polynucleotide encoding the trasnporter.

A fourth aspect relates to compositions comprising the yeast strain of the first aspect with one or more naturally occurring and/or non-naturally occurring components, such as components are selected from the group consisting of: surfactants, emulsifiers, gums, swelling agents, and antioxidants.

### BRIEF DESCRIPTION OF THE FIGURES

FIG. 1 shows improvement of tripeptide and tetrapeptide uptake by MBG4994 vs Ethanol Red® at 24 h during corn mash ethanol fermentation.

FIG. 2 shows final ethanol titer after 52 h fermentation of industrially prepared corn mash using the strains listed in Table 7.

FIG. 3 shows residual tripeptide after 29 h fermentation using industrially prepared corn mash with the strains listed in Table 7.

FIG. 4 shows residual tetrapeptide after 29 h fermentation using industrially prepared corn mash with the strains listed in Table 7.

FIG. 5 shows final ethanol titer after 53 h fermentation with industrially prepared mash using strains listed in Table 8.

FIG. 6 shows a plasmid map of pMBin369 described in Example 2.

FIG. 7 shows final ethanol titer after 68 h fermentation in corn mash with varied nitrogen concentration using Ethanol Red® (ER) and MBG4994.

### DEFINITIONS

Unless defined otherwise or clearly indicated by context, all technical and scientific terms used herein have the same meaning as commonly understood by one of ordinary skill in the art.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Auxiliary Activity 9: The term “Auxiliary Activity 9” or “AA9” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic-containing material by an enzyme having cellulolytic activity. Cellulolytic enhancing activity can be determined by measuring the increase in reducing sugars or the increase of the total of cellobiose and glucose from the hydrolysis of a cellulosic-containing material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5L (Novozymes A/S, Bagsværd, Denmark) and beta-glucosidase as the source of the cellulolytic activity, wherein the beta-glucosidase is present at a weight of at least 2-5% protein of the cellulase protein loading. In one embodiment, the beta-glucosidase is an Aspergillus oryzae beta-glucosidase (e.g., recombinantly produced in Aspergillus oryzae according to WO 02/095014). In another embodiment, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic-containing material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Catalase: The term “catalase” means a hydrogen-peroxide:hydrogen-peroxide oxidoreductase (EC 1.11.1.6) that catalyzes the conversion of 2 H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Catalytic domain: The term “catalytic domain” means the region of an enzyme containing the catalytic machinery of the enzyme.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., two, several) enzymes that hydrolyze a cellulosic-containing material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic-containing material by cellulolytic enzyme(s) under the following conditions: 1-50 mg of cellulolytic enzyme protein/g of cellulose in pretreated corn stover (PCS) (or other pretreated cellulosic-containing material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences that provide for its expression.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose, and may be the result of pretreating the source by enzymatic hydrolysis (saccharification). The term fermentation medium is understood herein to refer to a medium before the fermenting organism is added, such as, a medium resulting from a saccharification process, as well as a medium used in a simultaneous saccharification and fermentation process (SSF).

Hemicellulolytic enzyme or hemicellulase: The term “hemicellulolytic enzyme” or “hemicellulase” means one or more (e.g., two, several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, and the like with a nucleic acid construct or expression vector comprising a polynucleotide described herein (e.g., a polynucleotide encoding a trasporter, or regulator thereof). The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides introduced using recombinant techniques.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Pretreated corn stover: The term “Pretreated Corn Stover” or “PCS” means a cellulosic-containing material derived from corn stover by treatment with heat and dilute sulfuric acid, alkaline pretreatment, neutral pretreatment, or any pretreatment known in the art.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, California, including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Proteases may be endopeptidases (EC 3.4.21). Protease activity may be determined using methods described herein (See, Examples), known in the art (e.g., US 2015/0125925) or using commercially available assay kits (e.g., Sigma-Aldrich).

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Referenced Sequence−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylose Isomerase: The term “Xylose Isomerase” or “XI” means an enzyme which can catalyze D-xylose into D-xylulose in vivo, and convert D-glucose into D-fructose in vitro. Xylose isomerase is also known as “glucose isomerase” and is classified as E.C. 5.3.1.5.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value, and can include a range of plus or minus two standard deviations around the stated value.

Likewise, reference to a gene or polypeptide that is “derived from” another gene or polypeptide X, includes the gene or polypeptide X.

As used herein and in the appended claims, the singular forms “a,” “or,” and “the” include plural referents unless the context clearly dictates otherwise.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

### DETAILED DESCRIPTION

Described herein, inter alia, are methods for producing a fermentation product, such as ethanol, from starch or cellulosic containing material.

During industrial scale fermentation, yeast encounter various physiological challenges including variable concentrations of sugars, high concentrations of yeast metabolites such as ethanol, glycerol, organic acids, osmotic stress, as well as potential competition from contaminating microbes such as wild yeasts and bacteria. As a consequence, many yeasts are not suitable for use in industrial fermentation. The most widely used commercially available industrial strain of Saccharomyces (i.e. for industrial scale fermentation) is the Saccharomyces cerevisiae strain used, for example, in the product Ethanol Red®. This strain is well suited to industrial ethanol production; however, it requires significant amounts of added nitrogen, such urea and ammonia, to promote yeast growth.

The Applicant has developed yeast strains for ethanol fermentation that are capable of improved utilization of nitrogen, such as nitrogen from peptides (e.g., tripeptides/tetrapeptides) in the fermentation medium. Applicant's resulting yeast can be used in fermentation methods that provide fast rates and high yields without the dependence on large amounts of exogenously added protease and/or supplemental nitrogen source.

In one aspect is a method of producing a fermentation product from a starch-containing or cellulosic-containing material comprising:

(a) saccharifying the starch-containing or cellulosic-containing material; and
(b) fermenting the saccharified material of step (a) with a fermenting organism;

wherein the fermenting organism comprises a genetic modification that increases or decreases expression of transporter/permease or regulator thereof.

Steps a) and b) are carried out either sequentially or simultaneously (SSF). In one embodiment, steps a) and b) are carried out simultaneously (SSF). In another embodiment, steps a) and b) are carried out sequentially.

### Fermenting Organism

The fermenting organism described herein may be derived from any host cell known to the skilled artisan capable of producing a fermentation product, such as ethanol. As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cells for preparing the genetically modified cells described herein can be from any suitable host, such as a yeast strain, including, but not limited to, a Saccharomyces, Rhodotorula, Schizosaccharomyces, Kluyveromyces, Pichia, Hansenula, Issatchenkia, Rhodosporidium, Candida, Yarrowia, Lipomyces, Cryptococcus, or Dekkera sp. cell. In some embodiments, the yeast cell is a I. orientalis, C. lambica, S. bulderi or a S. cerevisiae cell. In particular, Saccharomyces host cells are contemplated, such as Saccharomyces cerevisiae, bayanus or carlsbergensis cells. In one embodiment, the yeast cell is a Saccharomyces cerevisiae cell.

Suitable cells can, for example, be derived from commercially available strains and polyploid or aneuploid industrial strains, including but not limited to those from Superstart™, THERMOSACC®, C5 FUEL™, XyloFerm®, etc. (Lallemand); RED STAR and ETHANOL RED® (Fermentis/Lesaffre, USA); FALI (AB Mauri); Baker's Best Yeast, Baker's Compressed Yeast, etc. (Fleishmann's Yeast); BIOFERM AFT, XP, CF, and XR (North American Bioproducts Corp.); Turbo Yeast (Gert Strand AB); and FERMIOL® (DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The genetic modifications may be introduced using methods known in the art and described herein, such as recombinant techniques, as well as non-recombinant breeding techniques (e.g., methods described and concerned in U.S. Pat. No. 8,257,959).

The strain may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference) or any strain described in WO2017/087330 (incorporated herein by reference).

The fermenting organisms according to the invention have been generated in order to improve fermentation yield and to improve process economy by cutting enzyme costs since part or all of the necessary enzymes needed to improve method performance are be produced by the fermenting organism.

The fermenting organisms described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the heterologous polynucleotide encoding the transporter is operably linked to a promoter foreign to the polynucleotide. The promoters may be identical to or share a high degree of sequence identity (e.g., at least about 80%, at least about 85%, at least about 90%, at least about 95%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, Journal of Bacteriology 177: 3465-3471).

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (See, e.g., Sambrook et al., 1989, supra).

Additional procedures and techniques known in the art for the preparation of recombinant cells for ethanol fermentation, are described in, e.g., WO2016/045569, the content of which is hereby incorporated by reference.

The fermenting organism may be in the form of a composition comprising a fermenting organism (e.g., a yeast strain described herein) and a naturally occurring and/or a non-naturally occurring component.

The fermenting organism described herein may be in any viable form, including crumbled, dry, including active dry and instant, compressed, cream (liquid) form etc. In one embodiment, the fermenting organism (e.g., a Saccharomyces cerevisiae yeast strain) is dry yeast, such as active dry yeast or instant yeast. In one embodiment, the fermenting organism (e.g., a Saccharomyces cerevisiae yeast strain) is crumbled yeast. In one embodiment, the fermenting organism (e.g., a Saccharomyces cerevisiae yeast strain) is compressed yeast. In one embodiment, the fermenting organism (e.g., a Saccharomyces cerevisiae yeast strain) is cream yeast.

In one embodiment is a composition comprising a fermenting organism described herein (e.g., a Saccharomyces cerevisiae yeast strain), and one or more of the component selected from the group consisting of: surfactants, emulsifiers, gums, swelling agent, and antioxidants and other processing aids.

The compositions described herein may comprise a fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

### Transporters/Permeases

In some embodiments, the fermenting organism (e.g., recombinant yeast cell) comprises a genetic modification that increases or decreases expression of a transporter/permease. The transporter may be any transporter that is suitable for improved nitrogen utilization of the fermenting organisms, such as a naturally occurring transporter (e.g., a native transporter from another species or an endogenous transporter expressed from a modified expression vector) or a variant thereof that retains transporter activity.

The transporters/permeases include, e.g., amino acid transporters, peptide transporters (such as any polypeptide capable of transporting dipeptides, tripeptides, and/or oligopeptides (n>3)), mitochondrial transporters, vacuole transporters, and ammonium permeases.

Transporter/permease activity can be measured using any suitable assay known in the art.

In some embodiments, the genetic modification is a heterologous polynucleotide encoding a transporter/permease.

In some embodiments, the fermenting organism has an increased level of transporter activity compared to the fermenting organism without the genetic modification, when cultivated under the same conditions. In some embodiments, the fermenting organism has an increased level of transporter activity of at least 5%, e.g., at least 10%, at least 15%, at least 20%, at least 25%, at least 50%, at least 100%, at least 150%, at least 200%, at least 300%, or at 500% compared to the fermenting organism without the genetic modification, when cultivated under the same conditions.

In some embodiments, the fermenting organism has increased or decreased expression of a transporter when compared to Saccharomyces cerevisiae strain Ethanol Red® (deposited under Accession No. V14/007039 at National Measurement Institute, Victoria, Australia) under the same conditions. In some embodiments, the fermenting organism has an increased expression of at least 5%, e.g., at least 10%, at least 15%, at least 20%, at least 25%, at least 50%, at least 100%, at least 150%, at least 200%, at least 300%, or at 500% compared to Saccharomyces cerevisiae strain Ethanol Red® (deposited under Accession No. V14/007039 at National Measurement Institute, Victoria, Australia) under the same conditions (e.g., under conditions described herein, such as on or after 53 hours fermentation).

Exemplary transporters that may be expressed with the fermenting organisms and methods of use described herein include, but are not limited to, transporters shown in Table 1 (or derivatives thereof).

TABLE 1

| | | | | Transporter |
|---|---|---|---|---|
| Gene Name | Description | Gene reference | Coding SEQ ID NO. | SEQ ID NO. |
| AGP1 | Amino Acid Permease | DAA07460.1 | 1 | 86 |
| BAP2 | Amino Acid Permease | CAA85012.1 | 2 | 87 |
| BAP3 | Amino Acid Permease | CAA52970.1 | 3 | 88 |
| GNP1 | Amino Acid Permease | AAB48002.1 | 4 | 89 |
| TAT1 | Amino Acid Permease | AAA50552.1 | 5 | 90 |
| TAT2 | Amino Acid Permease | CAA55777.1 | 6 | 91 |
| GAP1 | Amino Acid Permease | CAA36858.1 | 7 | 92 |
| HIP1 | Amino Acid Permease | AAA34673.1 | 8 | 93 |
| MMP1 | Amino Acid Permease | CAA97514.1 | 9 | 94 |
| SAM3 | Amino Acid Permease | CAA98010.1 | 10 | 95 |
| CAN1 | Amino Acid Permease | CAA27416.1 | 11 | 96 |
| ALP1 | Amino Acid Permease | CAA52199.1 | 12 | 97 |
| LYP1 | Amino Acid Permease | CAA47729.1 | 13 | 98 |
| PUT4 | Amino Acid Permease | AAA34925.1 | 14 | 99 |
| DIP5 | Amino Acid Permease | CAA65074.1 | 15 | 100 |
| SSY1 | Amino Acid Permease | CAA90380.1 | 16 | 101 |
| AGP2 | Amino Acid Permease | CAA53491.1 | 17 | 102 |
| AGP3 | Amino Acid Permease | BAA09186.1 | 18 | 103 |
| MUP1 | Amino Acid Permease | AAB63529.1 | 19 | 104 |
| MUP3 | Amino Acid Permease | AAB65048.1 | 20 | 105 |
| UGA4 | Amino Acid Permease | CAA47101.1 | 21 | 106 |
| TPO5 | Amino Acid Permease | CAA81512.1 | 22 | 107 |
| HNM1 | Amino Acid Permease | AAA34537.1 | 23 | 108 |
| BIO5 | Amino Acid Permease | AAB50012.1 | 24 | 109 |
| MEP1 | Ammonium Permease | CAA54699.1 | 25 | 110 |
| MEP2 | Ammonium Permease | CAA58587.1 | 26 | 111 |
| MEP3 | Ammonium Permease | AAB68278.1 | 27 | 112 |
| DAL5 | Other Transporters/Permease | AAA34555.1 | 28 | 113 |
| YCT1 | Other Transporters/Permease | CAA88002.1 | 29 | 114 |
| TNA1 | Other Transporters/Permease | CAA69082.1 | 30 | 115 |
| VHT1 | Other Transporters/Permease | CAA97067.1 | 31 | 116 |
| FEN2 | Other Transporters/Permease | CAA42320.1 | 32 | 117 |
| SEO1 | Other Transporters/Permease | AAC04968.1 | 33 | 118 |
| FUR4 | Other Transporters/Permease | CAA53678.1 | 34 | 119 |
| DAL4 | Other Transporters/Permease | CAA78826.1 | 35 | 120 |
| FUI1 | Other Transporters/Permease | CAA55059.1 | 36 | 121 |
| THI7 | Other Transporters/Permease | AAB67405.1 | 37 | 122 |
| THI72 | Other Transporters/Permease | CAA99401.1 | 38 | 123 |
| NRT1 | Other Transporters/Permease | CAA94556.1 | 39 | 124 |
| FCY2 | Other Transporters/Permease | CAA36040.1 | 40 | 125 |
| TPN1 | Other Transporters/Permease | CAA62788.1 | 41 | 126 |
| DUR3 | Other Transporters/Permease | AAA34582.1 | 42 | 127 |
| PTR2 | Other Transporters/Permease | AAC37368.1 | 43 | 128 |
| OPT1 | Other Transporters/Permease | CAA83999.1 | 44 | 129 |
| OPT2 | Other Transporters/Permease | AAB64623.1 | 45 | 130 |
| ATO3 | Other Transporters/Permease | AAB64820.1 | 46 | 131 |
| AQR1 | Other Transporters/Permease | AAA99646.1 | 47 | 132 |
| TPO1 | Other Transporters/Permease | CAA97477.1 | 48 | 133 |
| AGC1 | Mitochondrial Transporter | CAA95017.1 | 49 | 134 |
| ODC1 | Mitochondrial Transporter | AAB68225.1 | 50 | 135 |
| ODC2 | Mitochondrial Transporter | CAA63185.1 | 51 | 136 |
| ORT1 | Mitochondrial Transporter | CAA60862.1 | 52 | 137 |
| CTP1 | Mitochondrial Transporter | AAC48984.1 | 53 | 138 |
| OAC1 | Mitochondrial Transporter | AAA34886.1 | 54 | 139 |
| AVT5 | Mitochondrial Transporter | CAA56013.1 | 55 | 140 |
| LEU5 | Mitochondrial Transporter | AAB68424.1 | 56 | 141 |
| YMC1 | Mitochondrial Transporter | CAA47602.1 | 57 | 142 |
| YMC2 | Mitochondrial Transporter | CAA55607.1 | 58 | 143 |
| PET8 | Mitochondrial Transporter | AAA64802.1 | 59 | 144 |
| AVT1 | Vacuole Transporter | CAA60922.1 | 60 | 145 |
| AVT2 | Vacuole Transporter | AAB65023.1 | 61 | 146 |
| AVT3 | Vacuole Transporter | CAA81508.1 | 62 | 147 |
| AVT4 | Vacuole Transporter | CAA90525.1 | 63 | 148 |
| AVT5 | Vacuole Transporter | CAA56013.1 | 64 | 149 |
| AVT6 | Vacuole Transporter | AAC03217.1 | 65 | 150 |
| AVT7 | Vacuole Transporter | CAA86706.1 | 66 | 151 |
| VBA1 | Vacuole Transporter | CAA89235.1 | 67 | 152 |
| VBA2 | Vacuole Transporter | CAA85258.1 | 68 | 153 |
| VBA3 | Vacuole Transporter | CAA42398.1 | 69 | 154 |
| VBA4 | Vacuole Transporter | CAA88672.1 | 70 | 155 |
| VBA5 | Vacuole Transporter | CAA82185.1 | 71 | 156 |
| ATG22 | Vacuole Transporter | CAA42378.1 | 72 | 157 |
| CAN1 | Vacuole Transporter | CAA27416.1 | 73 | 158 |
| LYP1 | Vacuole Transporter | CAA47729.1 | 74 | 159 |
| RTC2 | Vacuole Transporter | CAA85105.1 | 75 | 160 |
| YGL114w | Other Transporters/Permease | CAA96822.1 | 76 | 161 |
| DUR3 | Urea and polyamine | AAA34582.1 | 77 | 162 |
| | transporter | | | |
| FOT2 | Other Transporters/Permease | CEP25277.1 | 78 | 163 |
| FOTX | Other Transporters/Permease | CEP25276.1 | 79 | 164 |
| OPT3 | Other Transporters/Permease | ABD17823.1 | 80 | 165 |
| OPT4 | Other Transporters/Permease | AOW30993.1 | 81 | 166 |
| OPT5 | Other Transporters/Permease | AOW27826.1 | 82 | 167 |
| OPT6 | Other Transporters/Permease | ABD17829.1 | 83 | 168 |
| OPT7 | Other Transporters/Permease | ABD17830.1 | 84 | 169 |
| OPT8 | Other Transporters/Permease | AOW30319.1 | 85 | 170 |

Additional polynucleotides encoding suitable transporters may be derived from microorganisms of any suitable genus, including those readily available within the UniProtKB database (www.uniprot.org).

The transporter may be a bacterial transporter. For example, the transporter may be derived from a Gram-positive bacterium such as a Bacillus, Clostridium, Enterococcus, Geobacillus, Lactobacillus, Lactococcus, Oceanobacillus, Staphylococcus, Streptococcus, or Streptomyces, or a Gram-negative bacterium such as a Campylobacter, E. coli, Flavobacterium, Fusobacterium, Helicobacter, Ilyobacter, Neisseria, Pseudomonas, Salmonella, or Ureaplasma.

In one embodiment, the transporter is derived from Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

In another embodiment, the transporter is derived from Streptococcus equisimilis, Streptococcus pyogenes, Streptococcus uberis, or Streptococcus equi subsp. Zooepidemicus.

In another embodiment, the transporter is derived from Streptomyces achromogenes, Streptomyces avermitilis, Streptomyces coelicolor, Streptomyces griseus, or Streptomyces lividans.

The transporter may be a fungal transporter. For example, the transporter may be derived from a yeast such as a Candida, Kluyveromyces, Pichia, Saccharomyces, Schizosaccharomyces, Yarrowia or Issatchenkia; or derived from a filamentous fungus such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, lrpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria.

In another embodiment, the transporter is derived from Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis.

In one embodiment, the transporter is derived from Torulaspora, such as the Torulaspora microellipsoides transporters of SEQ ID NO: 163 or SEQ ID NO: 164.

In another embodiment, the transporter is derived from Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The transporter coding sequences described or referenced herein, or a subsequence thereof, as well as the transporter described or referenced herein, or a fragment thereof, may be used to design nucleic acid probes to identify and clone DNA encoding a transporter from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with a coding sequence, or a subsequence thereof, the carrier material is used in a Southern blot.

In one embodiment, the nucleic acid probe is a polynucleotide, or subsequence thereof, that encodes the transporter of any one of SEQ ID NOs: 86-170, or a fragment thereof.

For purposes of the probes described above, hybridization indicates that the polynucleotide hybridizes to a labeled nucleic acid probe, or the full-length complementary strand thereof, or a subsequence of the foregoing; under very low to very high stringency conditions. Molecules to which the nucleic acid probe hybridizes under these conditions can be detected using, for example, X-ray film. Stringency and washing conditions are defined as described supra.

In one embodiment, the transporter is encoded by a polynucleotide that hybridizes under at least low stringency conditions, e.g., medium stringency conditions, medium-high stringency conditions, high stringency conditions, or very high stringency conditions with the full-length complementary strand of the coding sequence for any one of the transporters described or referenced herein (e.g., SEQ ID NOs: 1-85). (Sambrook et al., 1989, Molecular Cloning, A Laboratory Manual, 2d edition, Cold Spring Harbor, New York).

The transporter may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a transporter may then be derived by similarly screening a genomic or cDNA library of another microorganism or mixed DNA sample.

Once a polynucleotide encoding a transporter has been detected with a suitable probe as described herein, the sequence may be isolated or cloned by utilizing techniques that are known to those of ordinary skill in the art (See, e.g., Sambrook et al., 1989, supra). Techniques used to isolate or clone polynucleotides encoding transporters include isolation from genomic DNA, preparation from cDNA, or a combination thereof. The cloning of the polynucleotides from such genomic DNA can be effected, e.g., by using the well-known polymerase chain reaction (PCR) or antibody screening of expression libraries to detect cloned DNA fragments with shares structural features (See, e.g., Innis et al., 1990, PCR: A Guide to Methods and Application, Academic Press, New York). Other nucleic acid amplification procedures such as ligase chain reaction (LCR), ligated activated transcription (LAT) and nucleotide sequence-based amplification (NASBA) may be used.

In one embodiment, the transporter comprises or consists of the amino acid sequence of any one of SEQ ID NOs: 86-170 (such as SEQ ID NO: 129, SEQ ID NO: 163 or SEQ ID NO: 164). In another embodiment, the transporter is a fragment of the transporter of any one of SEQ ID NOs: 86-170, such as SEQ ID NO: 129, SEQ ID NO: 163 or SEQ ID NO: 164 (e.g., wherein the fragment has transporter activity). In one embodiment, the number of amino acid residues in the fragment is at least 75%, e.g., at least 80%, 85%, 90%, or 95% of the number of amino acid residues in referenced full length transporter (e.g. any one of SEQ ID NOs: 86-170; such as SEQ ID NO: 129, SEQ ID NO: 163 or SEQ ID NO: 164). In other embodiments, the transporter may comprise the catalytic domain of any transporter described or referenced herein (e.g., the catalytic domain of any one of SEQ ID NOs: 86-170; such as SEQ ID NO: 129, SEQ ID NO: 163 or SEQ ID NO: 164).

The transporter may be a variant of any one of the transporter described supra (e.g., any one of SEQ ID NOs: 86-170; such as SEQ ID NO: 129, SEQ ID NO: 163 or SEQ ID NO: 164). In one embodiment, the transporter has at least 60%, e.g., at least 65%, 70%, 75%, 80%, 85%, 90%, 95%, 97%, 98%, 99%, or 100% sequence identity to any one of the transporters described supra (e.g., any one of SEQ ID NOs: 86-170; such as SEQ ID NO: 129, SEQ ID NO: 163 or SEQ ID NO: 164).

In one embodiment, the transporter sequence differs by no more than ten amino acids, e.g., by no more than five amino acids, by no more than four amino acids, by no more than three amino acids, by no more than two amino acids, or by one amino acid from the amino acid sequence of any one of the transportera described supra (e.g., any one of SEQ ID NOs: 86-170; such as SEQ ID NO: 129, SEQ ID NO: 163 or SEQ ID NO: 164). In one embodiment, the transporter has an amino acid substitution, deletion, and/or insertion of one or more (e.g., two, several) of amino acid sequence of any one of the transporters described supra (e.g., any one of SEQ ID NOs: 86-170; such as SEQ ID NO: 129, SEQ ID NO: 163 or SEQ ID NO: 164). In some embodiments, the total number of amino acid substitutions, deletions and/or insertions is not more than 10, e.g., not more than 9, 8, 7, 6, 5, 4, 3, 2, or 1.

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R.L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the transporters, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site or other biological interaction can also be determined by physical analysis of structure, as determined by such techniques as nuclear magnetic resonance, crystallography, electron diffraction, or photoaffinity labeling, in conjunction with mutation of putative contact site amino acids (See, for example, de Vos et al., 1992, Science 255: 306-312; Smith et al., 1992, J. Mol. Biol. 224: 899-904; Wlodaver et al., 1992, FEBS Lett. 309: 59-64). The identities of essential amino acids can also be inferred from analysis of identities with other transporters that are related to the referenced transporter.

Additional guidance on the structure-activity relationship of the transporters herein can be determined using multiple sequence alignment (MSA) techniques well-known in the art. Based on the teachings herein, the skilled artisan could make similar alignments with any number of transporters described herein or known in the art. Such alignments aid the skilled artisan to determine potentially relevant domains (e.g., binding domains or catalytic domains), as well as which amino acid residues are conserved and not conserved among the different transporter sequences. It is appreciated in the art that changing an amino acid that is conserved at a particular position between disclosed polypeptides will more likely result in a change in biological activity (Bowie et al., 1990, Science 247: 1306-1310: “Residues that are directly involved in protein functions such as binding or catalysis will certainly be among the most conserved”). In contrast, substituting an amino acid that is not highly conserved among the polypeptides will not likely or significantly alter the biological activity.

Even further guidance on the structure-activity relationship for the skilled artisan can be found in published x-ray crystallography studies known in the art.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active transporters can be recovered from the host cells and rapidly sequenced using standard methods in the art. These methods allow the rapid determination of the importance of individual amino acid residues in a polypeptide.

In another embodiment, the heterologous polynucleotide encoding the transporter comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters described supra (e.g., any one of SEQ ID NOs: 1-85; such as SEQ ID NO: 44, SEQ ID NO: 78 or SEQ ID NO: 79).

In one embodiment, the heterologous polynucleotide encoding the transporter comprises or consists of the coding sequence of any one of the transporters described supra (e.g., any one of SEQ ID NOs: 1-85; such as SEQ ID NO: 44, SEQ ID NO: 78 or SEQ ID NO: 79). In another embodiment, the heterologous polynucleotide encoding the transporter comprises a subsequence of the coding sequence of any one of the transporters described supra (e.g., any one of SEQ ID NOs: 1-85; such as SEQ ID NO: 44, SEQ ID NO: 78 or SEQ ID NO: 79) wherein the subsequence encodes a polypeptide having transporter activity. In another embodiment, the number of nucleotides residues in the coding subsequence is at least 75%, e.g., at least 80%, 85%, 90%, or 95% of the number of the referenced coding sequence.

The referenced coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The transporter may be a fused polypeptide or cleavable fusion polypeptide in which another polypeptide is fused at the N-terminus or the C-terminus of the transporter. A fused polypeptide may be produced by fusing a polynucleotide encoding another polypeptide to a polynucleotide encoding the transporter. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

In some embodiments, the fermenting organism (e.g., recombinant yeast cell) comprises a disruption to an endogenous transporter gene (e.g., any one of the transporter genes shown in Table 1, such as any one of SEQ ID NOs: 1-85). In some embodiments, the disrupted endogenous transporter gene is inactivated. In another embodiment, the coding sequence of the endogenous gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters described supra (e.g., any one of SEQ ID NOs: 1-85). In another embodiment, the endogenous gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters described supra (e.g., any one of SEQ ID NOs: 86-170).

The fermenting organisms comprising a gene disruption may be constructed using methods well known in the art, including those methods described herein. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

The fermenting organisms comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The fermenting organisms comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229: 4719; Lo et al., 1985, Proc. Natl. Acad. Sci. U.S.A. 81: 2285; Higuchi et al., 1988, Nucleic Acids Res 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The fermenting organisms comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The fermenting organisms comprising a gene disruption may also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective gene.

The fermenting organisms comprising a gene disruption may be further constructed by random or specific mutagenesis using methods well known in the art, including, but not limited to, chemical mutagenesis (See, e.g., Hopwood, The Isolation of Mutants in Methods in Microbiology (J.R. Norris and D.W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one embodiment, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Regulators

In some embodiments, the fermenting organism (e.g., recombinant yeast cell) comprises a genetic modification that increases or decreases expression of a regulator, such as as tranporter regulator. Regulators may be any regulator that is suitable for improved nitrogen utilization of the fermenting organisms, such as a naturally occurring regulator (e.g., a native regulator from another species or an endogenous regulator expressed from a modified expression vector) or a variant thereof.

In some embodiments, the genetic modification is a heterologous polynucleotide encoding a regulator.

In some embodiments, the fermenting organism has increased or decreased expression of a regulator when compared to Saccharomyces cerevisiae strain Ethanol Red® (deposited under Accession No. V14/007039 at National Measurement Institute, Victoria, Australia) under the same conditions. In some embodiments, the fermenting organism has an increased expression of at least 5%, e.g., at least 10%, at least 15%, at least 20%, at least 25%, at least 50%, at least 100%, at least 150%, at least 200%, at least 300%, or at 500% compared to Saccharomyces cerevisiae strain Ethanol Red® (deposited under Accession No. V14/007039 at National Measurement Institute, Victoria, Australia) under the same conditions.

Exemplary regulators of regulators that may be expressed with the fermenting organisms and methods of use described herein include, but are not limited to, regulators shown in Table 2 below (or derivatives thereof).

TABLE 2
Regulators

| Gene | Gene | Coding | Regulator |
|---|---|---|---|
| Name | Reference | SEQ ID No. | SEQ ID No. |
| ABF1 | | 171 | 231 |
| ACE2 | | 172 | 232 |
| BUR6 | | 173 | 233 |
| CSE2 | | 174 | 234 |
| GAL11 | | 175 | 235 |
| GCN5 | | 176 | 236 |
| GCR1 | | 177 | 237 |
| HI R3 | | 178 | 238 |
| HSF1 | | 179 | 239 |
| IXR1 | | 180 | 240 |
| MED2 | | 181 | 241 |
| MET32 | | 182 | 242 |
| MET4 | | 183 | 243 |
| MSS11 | | 184 | 244 |
| OAF1 | | 185 | 245 |
| RPN4 | | 186 | 246 |
| RTG3 | | 187 | 247 |
| SFL1 | | 188 | 248 |
| SFP1 | | 189 | 249 |
| SK01 | | 190 | 250 |
| SPT20 | | 191 | 251 |
| SPT3 | | 192 | 252 |
| SPT7 | | 193 | 253 |
| SRB2 | | 194 | 254 |
| SRB8 | | 195 | 255 |
| SSN2 | | 196 | 256 |
| STP1 | | 197 | 257 |
| SWI4 | | 198 | 258 |
| TYE7 | | 199 | 259 |
| UGA3 | | 200 | 260 |
| UME6 | | 201 | 261 |
| YOX1 | | 202 | 262 |
| CDC28 | | 203 | 263 |
| YAPS | | 204 | 264 |
| YAP6 | | 205 | 265 |
| CST6 | | 206 | 266 |
| FHL1 | | 207 | 267 |
| GCN4 | AAA34640.1 | 208 | 268 |
| LEU3 | | 209 | 269 |
| MET31 | | 210 | 270 |
| SPN1 | | 211 | 271 |
| SPT6 | | 212 | 272 |
| TFC7 | | 213 | 273 |
| TUP1 | | 214 | 274 |
| VVTM1 | | 215 | 275 |
| YAP1 | | 216 | 276 |
| YAP6 | | 217 | 277 |
| CAD1 | | 218 | 278 |
| XBP1 | | 219 | 279 |
| GLN3 | | 220 | 280 |
| RGR1 | | 221 | 281 |
| IXR1 | | 222 | 282 |
| RTC1 | CAA64732.1 | 223 | 283 |
| GAT1 | AAB03516.1 | 224 | 284 |
| CUP9 | AAA66189.1 | 225 | 285 |
| NPR2 | DAA07592.1 | 226 | 286 |
| DAL81 | AAA35192.1 | 227 | 287 |
| DAL82 | CAA38391.1 | 228 | 288 |
| PTR1 | CAA37779.1 | 229 | 289 |
| PTR3 | BAA09268.1 | 230 | 290 |

Additional polynucleotides encoding suitable regulators may be derived from microorganisms of any suitable genus, including those readily available within the UniProtKB database (www.uniprot.org).

The regulator may be a regulator from any bacterial or fungal species, as described supra.

The regulator coding sequences described or referenced herein, or a subsequence thereof, as well as the regulators described or referenced herein, or a fragment thereof, may be used to design nucleic acid probes to identify and clone DNA encoding a regulators from strains of different genera or species as described supra. In one embodiment, the nucleic acid probe is a polynucleotide, or subsequence thereof, that encodes the regulator of any one of SEQ ID NOs: 231-290, or a fragment thereof.

In one embodiment, the regulator is encoded by a polynucleotide that hybridizes under at least low stringency conditions, e.g., medium stringency conditions, medium-high stringency conditions, high stringency conditions, or very high stringency conditions with the full-length complementary strand of the coding sequence for any one of the regulator described or referenced herein (e.g., SEQ ID NOs: 171-230). (Sambrook et al., 1989, Molecular Cloning, A Laboratory Manual, 2d edition, Cold Spring Harbor, New York).

The regulator may also be identified and obtained from other sources including microorganisms isolated from nature (e.g., soil, composts, water, silage, etc.) or DNA samples obtained directly from natural materials (e.g., soil, composts, water, silage, etc.) as described supra.

Once a polynucleotide encoding a regulator has been detected with a suitable probe as described herein, the sequence may be isolated or cloned by utilizing techniques that are known to those of ordinary skill in the art, as described supra.

In one embodiment, the regulator is a polypeptide that regulates any one of the transporters of Table 1.

In one embodiment, the regulator comprises or consists of the amino acid sequence of any one of SEQ ID NOs: 231-290. In another embodiment, the transporter is a fragment of the regulator of any one of SEQ ID NOs: 231-290. In one embodiment, the number of amino acid residues in the fragment is at least 75%, e.g., at least 80%, 85%, 90%, or 95% of the number of amino acid residues in referenced full-length regulator (e.g., any one of SEQ ID NOs: 231-290).

The regulator may be a variant of any one of the regulators described supra (e.g., any one of SEQ ID NOs: 231-290). In one embodiment, the regulator has at least 60%, e.g., at least 65%, 70%, 75%, 80%, 85%, 90%, 95%, 97%, 98%, 99%, or 100% sequence identity to any one of the regulators described supra (e.g., any one of SEQ ID NOs: 231-290).

In one embodiment, the regulator sequence differs by no more than ten amino acids, e.g., by no more than five amino acids, by no more than four amino acids, by no more than three amino acids, by no more than two amino acids, or by one amino acid from the amino acid sequence of any one of the regulator described supra (e.g., any one of SEQ ID NOs: 231-290). In one embodiment, the regulator has an amino acid substitution, deletion, and/or insertion of one or more (e.g., two, several) of amino acid sequence of any one of the regulators described supra (e.g., any one of SEQ ID NOs: 231-290). In some embodiments, the total number of amino acid substitutions, deletions and/or insertions is not more than 10, e.g., not more than 9, 8, 7, 6, 5, 4, 3, 2, or 1.

In another embodiment, the heterologous polynucleotide encoding the regulator comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulators described supra (e.g., any one of SEQ ID NOs: 171-230).

In one embodiment, the heterologous polynucleotide encoding the regulator comprises or consists of the coding sequence of any one of the regulators described supra (e.g., any one of SEQ ID NOs: 171-230). In another embodiment, the heterologous polynucleotide encoding the regulator comprises a subsequence of the coding sequence of any one of the regulators described supra (e.g., any one of SEQ ID NOs: 171-230). In another embodiment, the number of nucleotides residues in the coding subsequence is at least 75%, e.g., at least 80%, 85%, 90%, or 95% of the number of the referenced coding sequence.

The referenced coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The regulator may be a fused polypeptide or cleavable fusion polypeptide, as described supra. 1993, EMBO J. 12: 2575-2583; Dawson et al., 1994, Science 266: 776-779).

In some embodiments, the fermenting organism (e.g., recombinant yeast cell) comprises a disruption to an endogenous regulator gene (e.g., any one of the regulator genes shown in Table 2, such as any one of SEQ ID NOs: 171-230). In some embodiments, the disrupted endogenous regulator gene is inactivated. In another embodiment, the coding sequence of the endogenous gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulators described supra (e.g., any one of SEQ ID NOs: 171-230). In another embodiment, the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators described supra (e.g., any one of SEQ ID NOs: 231-290). Methods of gene disruption are described supra.

### Additional Gene Disruptions

The fermenting organisms described herein may also comprise one or more (e.g., two, several) gene disruptions, e.g., to divert sugar metabolism from undesired products to ethanol. In some aspects, the recombinant host cells produce a greater amount of ethanol compared to the cell without the one or more disruptions when cultivated under identical conditions. In some aspects, one or more of the disrupted endogenous genes is inactivated.

In certain embodiments, the recombinant cells provided herein comprise a disruption of one or more endogenous genes encoding enzymes involved in producing alternate fermentative products such as glycerol or other byproducts such as acetate or diols. For example, the cells provided herein may comprise a disruption of one or more of glycerol 3-phosphate dehydrogenase (GPD, catalyzes reaction of dihydroxyacetone phosphate to glycerol 3-phosphate), glycerol 3-phosphatase (GPP, catalyzes conversion of glycerol-3 phosphate to glycerol), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), and aldehyde dehydrogenase (ALD, e.g., converts acetaldehyde to acetate). Disruptions to GPD1/GPD2 and GPP1/GPP2 are discussed in, e.g., WO2014/180820 (the seqeuences of which are incorporated herein by reference). In some embodiments, the recombinant cells provided herein comprise a disruption to an aldose reductase (catalyzes conversion of xylose or xylulose to xylitol; e.g., GRE3 or YPR1; See, Traff et al., 2001, Appl. Environ. Microbiol. 67: 5668-74).

Modeling analysis can be used to design gene disruptions that additionally optimize utilization of the pathway. One exemplary computational method for identifying and designing metabolic alterations favoring biosynthesis of a desired product is the OptKnock computational framework, Burgard et al., 2003, Biotechnol. Bioeng. 84: 647-657.

The fermenting organisms comprising a gene disruption may be constructed using methods well known in the art, such as those described supra.

### Methods Using a Starch-Containing Material

In some aspects, the methods described herein produce a fermentation product from a starch-containing material. Starch-containing material is well-known in the art, contining two typs of homopolysaccharides (amylose and amylopectin) and is linked by alpha-(1-4)-D-glycosidic bonds. Any suitable starch-containing starting material may be used. The starting material is generally selected based on the desired fermentation product, such as ethanol. Examples of starch-containing starting materials include cereal, tubers or grains. Specifically, the starch-containing material may be corn, wheat, barley, rye, milo, sago, cassava, tapioca, sorghum, oat, rice, peas, beans, or sweet potatoes, or mixtures thereof. Contemplated are also waxy and non-waxy types of corn and barley.

In one embodiment, the starch-containing starting material is corn. In one embodiment, the starch-containing starting material is wheat. In one embodiment, the starch-containing starting material is barley. In one embodiment, the starch-containing starting material is rye. In one embodiment, the starch-containing starting material is milo. In one embodiment, the starch-containing starting material is sago. In one embodiment, the starch-containing starting material is cassava. In one embodiment, the starch-containing starting material is tapioca. In one embodiment, the starch-containing starting material is sorghum. In one embodiment, the starch-containing starting material is rice. In one embodiment, the starch-containing starting material is peas. In one embodiment, the starch-containing starting material is beans. In one embodiment, the starch-containing starting material is sweet potatoes. In one embodiment, the starch-containing starting material is oats.

The methods using a starch-containing material may include a conventional process (e.g., including a liquefaction step described in more detail below) or a raw starch hydrolysis process. In some embodiments using a starch-containing material, saccarification of the starch-containing material is at a temperature above the initial gelatinization temperature. In some embodiments using a starch-containing material, saccarification of the starch-containing material is at a temperature below the initial gelatinization temperature.

### Liquefaction

In aspects using a starch-containing material, the methods may further comprise a liquefaction step carried out by subjecting the starch-containing material at a temperature above the initial gelatinization temperature to an alpha-amylase and optionally a protease and/or a glucoamylase. Other enzymes such as a pullulanase, endoglucanase, hemicellulase (e.g., xylanase), phospholipase C, and phytase may also be present and/or added in liquefaction. In some embodiments, the liquefaction step is carried out prior to steps a) and b) of the described methods.

Liquefaction step may be carried out for 0.5-5 hours, such as 1-3 hours, such as typically about 2 hours.

The term “initial gelatinization temperature” means the lowest temperature at which gelatinization of the starch-containing material commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. The initial gelatinization temperature of a given starch-containing material may be determined as the temperature at which birefringence is lost in 5% of the starch granules using the method described by Gorinstein and Lii, 1992, Starch/Stärke 44(12): 461-466.

Liquefaction is typically carried out at a temperature in the range from 70-100° C. In one embodiment, the temperature in liquefaction is between 75-95° C., such as between 75-90° C., between 80-90° C., or between 82-88° C., such as about 85° C. In one embodiment, the temperature in liquefaction is greater than 85° C., such as about 88° C., about 89° C., about 90° C., about 91° C., about 92° C., about 93° C., about 94 ° C., or about 95° C.

A jet-cooking step may be carried out prior to liquefaction in step, for example, at a temperature between 110-145° C., 120-140° C., 125-135° C., or about 130° C. for about 1-15 minutes, for about 3-10 minutes, or about 5 minutes.

The pH during liquefaction may be e.g., between 4 and 7, such as pH 4.5-6.5, pH 5.0-6.5, pH 5.0-6.0, pH 5.2-6.2, or about 5.2, about 5.4, about 5.6, or about 5.8.

In one embodiment, the process further comprises, prior to liquifaction, the steps of:
i) reducing the particle size of the starch-containing material, preferably by dry milling;
ii) forming a slurry comprising the starch-containing material and water.

The starch-containing starting material, such as whole grains, may be reduced in particle size, e.g., by milling, in order to open up the structure, to increase surface area, and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry milling and wet milling are well known in the art of starch processing. In one embodiment the starch-containing material is subjected to dry milling. In one embodiment, the particle size is reduced to between 0.05 to 3.0 mm, e.g., 0.1-0.5 mm, or so that at least 30%, at least 50%, at least 70%, or at least 90% of the starch-containing material fit through a sieve with a 0.05 to 3.0 mm screen, e.g., 0.1-0.5 mm screen. In another embodiment, at least 50%, e.g., at least 70%, at least 80%, or at least 90% of the starch-containing material fit through a sieve with #6 screen.

The aqueous slurry may contain from 10-55 w/w-% dry solids (DS), e.g., 25-45 w/w-% dry solids (DS), or 30-40 w/w-% dry solids (DS) of starch-containing material.

The alpha-amylase, optionally a protease, and optionally a glucoamylase may initially be added to the aqueous slurry to initiate liquefaction (thinning). In one embodiment, only a portion of the enzymes (e.g., about ⅓) is added to the aqueous slurry, while the rest of the enzymes (e.g., about ⅔) are added during liquefaction step.

A non-exhaustive list of alpha-amylases used in liquefaction can be found below in the “Alpha-Amylases” section. Examples of suitable proteases used in liquefaction include any protease described in the “Proteases” section. Examples of suitable glucoamylases used in liquefaction include any glucoamylase found in the “Glucoamylases in Liquefaction” section.

### Alpha-Amylases

An alpha-amylase may be present and/or added in liquefaction optionally together with a protease, phytase, endoglucase, phospholipase C, xylanase, glucoamylase, and/or pullulanase, e.g., as disclosed in WO2012/088303 (Novozymes) or WO2013/082486 (Novozymes) which references are both incorporated by reference.

In some embodiments, the fermenting organism comprises a heterologous polynucleotide encoding an alpha-amylase, for example, as described in WO2017/087330, the content of which is hereby incorporated by reference. Any alpha-amylase described or referenced herein is contemplated for expression in the fermenting organism.

The alpha-amylase may be any alpha-amylase that is suitable for the host cells and/or the methods described herein, such as a naturally occurring alpha-amylase or a variant thereof that retains alpha-amylase activity.

In some embodiments, the fermenting organism comprising a heterologous polynucleotide encoding an alpha-amylase has an increased level of alpha-amylase activity compared to the host cells without the heterologous polynucleotide encoding the alpha-amylase, when cultivated under the same conditions. In some embodiments, the fermenting organism has an increased level of alpha-amylase activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the alpha-amylase, when cultivated under the same conditions.

Exemplary alpha-amylases that can be used with the host cells and/or the methods described herein include bacterial, yeast, or filamentous fungal alpha-amylases, e.g., derived from any of the microorganisms described or referenced herein.

The term “bacterial alpha-amylase” means any bacterial alpha-amylase classified under EC 3.2.1.1. A bacterial alpha-amylase used herein may, e.g., be derived from a strain of the genus Bacillus, which is sometimes also referred to as the genus Geobacillus. In one embodiment, the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase (BSG) of SEQ ID NO: 3 in WO99/19467, the Bacillus amyloliquefaciens alpha-amylase (BAN) of SEQ ID NO: 5 in WO99/19467, and the Bacillus licheniformis alpha-amylase (BLA) of SEQ ID NO: 4 in WO99/19467 (all sequences are hereby incorporated by reference). In one embodiment, the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO99/19467.

In one embodiment, the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO99/19467.

In one embodiment, the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated at the C-terminal, so that it is from 480-495 amino acids long, such as about 491 amino acids long, e.g., so that it lacks a functional starch binding domain (compared to SEQ ID NO: 3 in WO99/19467).

The Bacillus alpha-amylase may also be a variant and/or hybrid. Examples of such a variant can be found in any of WO96/23873, WO96/23874, WO97/41213, WO99/19467, WO00/60059, and WO02/10355 (each hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO96/23873—See, e.g., page 20, lines 1-10 (hereby incorporated by reference), such as corresponding to deletion of positions I181 and G182 compared to the amino acid sequence of Bacillus stearothermophilus alpha-amylase set forth in SEQ ID NO: 3 disclosed in WO99/19467 or the deletion of amino acids R179 and G180 using SEQ ID NO: 3 in WO99/19467 for numbering (which reference is hereby incorporated by reference). In some embodimenst, the Bacillus alpha-amylases, such as Bacillus stearothermophilus alpha-amylases, have a double deletion corresponding to a deletion of positions 181 and 182 and further optionally comprise a N193F substitution (also denoted I181*+G182*+N193F) compared to the wild-type BSG alpha-amylase amino acid sequence set forth in SEQ ID NO: 3 disclosed in WO 99/19467. The bacterial alpha-amylase may also have a substitution in a position corresponding to S239 in the Bacillus licheniformis alpha-amylase shown in SEQ ID NO: 4 in WO99/19467, or a S242 and/or E188P variant of the Bacillus stearothermophilus alpha-amylase of SEQ ID NO: 3 in WO99/19467.

In one embodiment, the variant is a S242A, E or Q variant, e.g., a S242Q variant, of the Bacillus stearothermophilus alpha-amylase.

In one embodiment, the variant is a position E188 variant, e.g., E188P variant of the Bacillus stearothermophilus alpha-amylase.

The bacterial alpha-amylase may, in one embodiment, be a truncated Bacillus alpha-amylase. In one embodiment, the truncation is so that, e.g., the Bacillus stearothermophilus alpha-amylase shown in SEQ ID NO: 3 in WO99/19467, is about 491 amino acids long, such as from 480 to 495 amino acids long, or so it lacks a functional starch bind domain.

The bacterial alpha-amylase may also be a hybrid bacterial alpha-amylase, e.g., an alpha-amylase comprising 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467). In one embodiment, this hybrid has one or more, especially all, of the following substitutions: G48A+T49I+G107A+H156Y+A181T+N190F+I201F+A209V+Q264S (using the Bacillus licheniformis numbering in SEQ ID NO: 4 of WO 99/19467). In some embodiments, the variants have one or more of the following mutations (or corresponding mutations in other Bacillus alpha-amylases): H154Y, A181T, N190F, A209V and Q264S and/or the deletion of two residues between positions 176 and 179, e.g., deletion of E178 and G179 (using SEQ ID NO: 5 of WO 99/19467 for position numbering).

In one embodiment, the bacterial alpha-amylase is the mature part of the chimeric alpha-amylase disclosed in Richardson et al. (2002), The Journal of Biological Chemistry, Vol. 277, No 29, Issue 19 July, pp. 267501-26507, referred to as BD5088 or a variant thereof. This alpha-amylase is the same as the one shown in SEQ ID NO: 2 in WO2007/134207. The mature enzyme sequence starts after the initial “Met” amino acid in position 1.

The alpha-amylase may be a thermostable alpha-amylase, such as a thermostable bacterial alpha-amylase, e.g., from Bacillus stearothermophilus. In one embodiment, the alpha-amylase used in a process described herein has a T½ (min) at pH 4.5, 85° C., 0.12 mM CaCl₂ of at least 10 determined as described in Example 1 of WO2018/098381.

In one embodiment, the thermostable alpha-amylase has a T½ (min) at pH 4.5, 85° C., 0.12 mM CaCl₂, of at least 15. In one embodiment, the thermostable alpha-amylase has a T½ (min) at pH 4.5, 85° C., 0.12 mM CaCl₂, of as at least 20. In one embodiment, the thermostable alpha-amylase has a T½ (min) at pH 4.5, 85° C., 0.12 mM CaCl₂, of as at least 25. In one embodiment, the thermostable alpha-amylase has a T½ (min) at pH 4.5, 85° C., 0.12 mM CaCl₂, of as at least 30. In one embodiment, the thermostable alpha-amylase has a T½ (min) at pH 4.5, 85° C., 0.12 mM CaCl₂, of as at least 40.

In one embodiment, the thermostable alpha-amylase has a T½ (min) at pH 4.5, 85° C., 0.12 mM CaCl₂, of at least 50. In one embodiment, the thermostable alpha-amylase has a T½ (min) at pH 4.5, 85° C., 0.12 mM CaCl₂, of at least 60. In one embodiment, the thermostable alpha-amylase has a T½ (min) at pH 4.5, 85° C., 0.12 mM CaCl₂, between 10-70. In one embodiment, the thermostable alpha-amylase has a T½ (min) at pH 4.5, 85° C., 0.12 mM CaCl₂, between 15-70. In one embodiment, the thermostable alpha-amylase has a T½ (min) at pH 4.5, 85° C., 0.12 mM CaCl₂, between 20-70. In one embodiment, the thermostable alpha-amylase has a T½ (min) at pH 4.5, 85° C., 0.12 mM CaCl₂, between 25-70. In one embodiment, the thermostable alpha-amylase has a T½ (min) at pH 4.5, 85° C., 0.12 mM CaCl₂, between 30-70. In one embodiment, the thermostable alpha-amylase has a T½ (min) at pH 4.5, 85° C., 0.12 mM CaCl₂, between 40-70. In one embodiment, the thermostable alpha-amylase has a T½ (min) at pH 4.5, 85° C., 0.12 mM CaCl₂, between 50-70. In one embodiment, the thermostable alpha-amylase has a T½ (min) at pH 4.5, 85° C., 0.12 mM CaCl₂, between 60-70.

In one embodiment, the alpha-amylase is a bacterial alpha-amylase, e.g., derived from the genus Bacillus, such as a strain of Bacillus stearothermophilus, e.g., the Bacillus stearothermophilus as disclosed in WO 99/019467 as SEQ ID NO: 3 with one or two amino acids deleted at positions R179, G180, I181 and/or G182, in particular with R179 and G180 deleted, or with I181 and G182 deleted, with mutations in below list of mutations.

In some embodiment, the Bacillus stearothermophilus alpha-amylases have double deletion I181+G182, and optional substitution N193F, further comprising one of the following substitutions or combinations of substitutions:

V59A+Q89R+G112 D+E129V+K177L+R179E+K220P+N224L+Q254S;

V59A+Q89R+E129V+K177L+R179E+H208Y+K220P+N224L+Q254S;

V59A+Q89R+E129V+K177L+R179E+K220P+N224L+Q254S+D269E+D281N;

V59A+Q89R+E129V+K177L+R179E+K220P+N224L+Q254S+I270L;

V59A+Q89R+E129V+K177L+R179E+K220P+N224L+Q254S+H274K;

V59A+Q89R+E129V+K177L+R179E+K220P+N224L+Q254S+Y276F;

V59A+E129V+R157Y+K177L+R179E+K220P+N224L+S242Q+Q254S;

V59A+E129V+K177L+R179E+H208Y+K220P+N224L+S242Q+Q254S;

V59A+E129V+K177L+R179E+K220P+N224L+S242Q+Q254S;

V59A+E129V+K177L+R179E+K220P+N224L+S242Q+Q254S+H274K;

V59A+E129V+K177L+R179E+K220P+N224L+S242Q+Q254S+Y276F;

V59A+E129V+K177L+R179E+K220P+N224L+S242Q+Q254S+D281N;

V59A+E129V+K177L+R179E+K220P+N224L+S242Q+Q254S+M284T;

V59A+E129V+K177L+R179E+K220P+N224L+S242Q+Q254S+G416V;

V59A+E129V+K177L+R179E+K220P+N224L+Q254S;

V59A+E129V+K177L+R179E+K220P+N224L+Q254S+M284T;

A91L+M96I+E129V+K177L+R179E+K220P+N224L+S242Q+Q254S;

E129V+K177L+R179E;

E129V+K177L+R179E+K220P+N224L+S242Q+Q254S;

E129V+K177L+R179E+K220P+N224L+S242Q+Q254S+Y276F+L427M;

E129V+K177L+R179E+K220P+N224L+S242Q+Q254S+M284T;

E129V+K177L+R179E+K220P+N224L+5242Q+Q2545+N376*+1377*;

E129V+K177L+R179E+K220P+N224L+Q254S;

E129V+K177L+R179E+K220P+N224L+Q254S+M284T;

E129V+K177L+R179E+S242Q;

E129V+K177L+R179V+K220P+N224L+S242Q+Q254S;

K220P+N224L+S242Q+Q254S;

M284V;

V59A+Q89R+E129V+K177L+R179E+Q254S+M284V; and

V59A+E129V+K177L+R179E+Q254S+M284V;

In one embodiment, the alpha-amylase is selected from the group of Bacillus stearothermophilus alpha-amylase variants with double deletion I181*+G182*, and optionally substitution N193F, and further one of the following substitutions or combinations of substitutions:

E129V+K177L+R179E;

V59A+Q89R+E129V+K177L+R179E+H208Y+K220P+N224L+Q254S;

V59A+Q89R+E129V+K177L+R179E+Q254S+M284V;

V59A+E129V+K177L+R179E+Q254S+M284V; and

E129V+K177L+R179E+K220P+N224L+S242Q+Q254S (using SEQ ID NO: 1 herein for numbering).

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467, or variants thereof, are truncated in the C-terminal and are typically from 480-495 amino acids long, such as about 491 amino acids long, e.g., so that it lacks a functional starch binding domain.

In one embodiment, the alpha-amylase variant may be an enzyme having a degree of identity of at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467.

In one embodiment, the bacterial alpha-amylase, e.g., Bacillus alpha-amylase, such as especially Bacillus stearothermophilus alpha-amylase, or variant thereof, is dosed to liquefaction in a concentration between 0.01-10 KNU-A/g DS, e.g., between 0.02 and 5 KNU-A/g DS, such as 0.03 and 3 KNU-A, preferably 0.04 and 2 KNU-A/g DS, such as especially 0.01 and 2 KNU-A/g DS. In one embodiment, the bacterial alpha-amylase, e.g., Bacillus alpha-amylase, such as especially Bacillus stearothermophilus alpha-amylases, or variant thereof, is dosed to liquefaction in a concentration of between 0.0001-1 mg EP (Enzyme Protein)/g DS, e.g., 0.0005-0.5 mg EP/g DS, such as 0.001-0.1 mg EP/g DS.

In one embodiment, the bacterial alpha-amylase is derived from a Bacillus subtilis alpha-amylase (SEQ ID NOs: 76, 83 or 84 of WO2018/222990), a Bacillus subtilis alpha-amylase (SEQ ID NO: 82 of WO2018/222990), a Bacillus licheniformis alpha-amylase (SEQ ID NO: 85 of WO2018/222990), a Clostridium phytofermentans alpha-amylase (SEQ ID NOs: 89-94 of WO2018/222990), a Clostridium thermocellum alpha-amylase (SEQ ID NO: 95 of WO2018/222990), a Thermobifida fusca alpha-amylase (SEQ ID NOs: 96 or 97 of WO2018/222990), a Thermobifida fusca alpha-amylase (SEQ ID NO: 97 or of WO2018/222990), a Anaerocellum thermophilum (SEQ ID NOs: 98, 99, or 100 of WO2018/222990), or a Streptomyces avermitilis alpha amylase (SEQ ID NO: 88 or 101 of WO2018/222990).

In one embodiment, the alpha-amylase is derived from a yeast alpha-amylase, such as the Saccharomycopsis fibuligera alpha-amylase (SEQ ID NO: 77 of WO2018/222990), a Debaryomyces occidentalis alpha-amylase (SEQ ID NOs: 78 or 79 of WO2018/222990), or a Lipomyces kononenkoae alpha-amylase (SEQ ID NO: 80 or 81 of WO2018/222990).

In one embodiment, the alpha-amylase is derived from a filamentous fungal alpha-amylase, such as an Aspergillus niger alpha-amylase (SEQ ID NO: 86 or 87 of WO2018/222990).

Additional alpha-amylases contemplated for use with the present invention can be found in WO2011/153516 (the content of which is incorporated herein).

Additional polynucleotides encoding suitable alpha-amylases may be obtained from microorganisms of any genus, including those readily available within the UniProtKB database (www.uniprot.org).

The alpha-amylase coding sequences can also be used to design nucleic acid probes to identify and clone DNA encoding alpha-amylases from strains of different genera or species, as described supra.

The polynucleotides encoding alpha-amylases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding alpha-amylases are described supra.

In one embodiment, the alpha-amylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any alpha-amylase described or referenced herein (e.g., the Debaryomyces occidentalis alpha-amylase shown as SEQ ID NO: 79 of WO2018/222990). In one aspect, the alpha-amylase sequence differs by no more than ten amino acids, e.g., by no more than five amino acids, by no more than four amino acids, by no more than three amino acids, by no more than two amino acids, or by one amino acid from any alpha-amylase described or referenced herein (e.g., the Debaryomyces occidentalis alpha-amylase shown as SEQ ID NO: 79 of WO2018/222990). In one embodiment, the alpha-amylase comprises or consists of the amino acid sequence of any alpha-amylase described or referenced herein (e.g., the Debaryomyces occidentalis alpha-amylase shown as SEQ ID NO: 79 of WO2018/222990), allelic variant, or a fragment thereof having alpha-amylase activity. In one embodiment, the alpha-amylase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the alpha-amylase has at least 20%, e.g., at least 40%, at least 50%, at least 60%, at least 70%, at least 80%, at least 90%, at least 95%, at least 96%, at least 97%, at least 98%, at least 99%, or 100% of the alpha-amylase activity of any alpha-amylase described or referenced herein (e.g., the Debaryomyces occidentalis alpha-amylase shown as SEQ ID NO: 79 of WO2018/222990) under the same conditions.

In one embodiment, the alpha-amylase coding sequence hybridizes under at least low stringency conditions, e.g., medium stringency conditions, medium-high stringency conditions, high stringency conditions, or very high stringency conditions with the full-length complementary strand of the coding sequence from any alpha-amylase described or referenced herein (e.g., the Debaryomyces occidentalis alpha-amylase shown as SEQ ID NO: 79 of WO2018/222990). In one embodiment, the alpha-amylase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any alpha-amylase described or referenced herein (e.g., the Debaryomyces occidentalis alpha-amylase shown as SEQ ID NO: 79 of WO2018/222990).

In one embodiment, the polynucleotide encoding the alpha-amylase comprises the coding sequence of any alpha-amylase described or referenced herein (e.g., the Debaryomyces occidentalis alpha-amylase shown as SEQ ID NO: 79 of WO2018/222990). In one embodiment, the polynucleotide encoding the alpha-amylase comprises a subsequence of the coding sequence from any alpha-amylase described or referenced herein, wherein the subsequence encodes a polypeptide having alpha-amylase activity. In one embodiment, the number of nucleotides residues in the subsequence is at least 75%, e.g., at least 80%, 85%, 90%, or 95% of the number of the referenced coding sequence.

The alpha-amylase can also include fused polypeptides or cleavable fusion polypeptides, as described supra.

### Proteases

In the processes described herein, a protease may optionally be present and/or added in slurry and/or liquefaction together with alpha-amylase, and an optional glucoamylase, phospholipase C, xylanase, endoglucanase, phytase, and/or pullulanase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In some embodiments, the fermenting organism comprises a heterologous polynucleotide encoding a protease, for example, as described in WO2018/222990, the content of which is hereby incorporated by reference. Any protease described or referenced herein is contemplated for expression in the fermenting organism.

The protease may be any protease that is suitable for the host cells and/or the methods described herein, such as a naturally occurring protease or a variant thereof that retains protease activity.

In some embodiments, the fermenting organism comprising a heterologous polynucleotide encoding a protease has an increased level of protease activity compared to the host cells without the heterologous polynucleotide encoding the protease, when cultivated under the same conditions. In some embodiments, the fermenting organism has an increased level of protease activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the protease, when cultivated under the same conditions.

Exemplary proteases that can be used with the host cells and/or the methods described herein include bacterial, yeast, or filamentous fungal proteases, e.g., derived from any of the microorganisms described or referenced herein.

In one embodiment, the thermostable protease used according to a process described herein is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein).

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu.

There are no limitations on the origin of the protease used in a process described herein as long as it fulfills the thermostability properties defined below.

In one embodiment the protease is of fungal origin.

The protease may be a variant of, e.g., a wild-type protease as long as the protease has the thermostability properties defined herein. In one embodiment, the thermostable protease is a variant of a metallo protease as defined above. In one embodiment, the thermostable protease used in a process described herein is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In one embodiment, the thermostable protease is a variant of the mature part of the metallo protease shown in SEQ ID NO: 2 disclosed in WO2003/048353 or the mature part of SEQ ID NO: 1 in WO2010/008841 (SEQ ID NO: 292 herein) further with one of the following substitutions or combinations of substitutions:

S5*+D79L+S87P+A112P+D142L;

D79L+S87P+A112P+T124V+D142L;

S5*+N26R+D79L+S87P+A112P+D142L;

N26R+T46R+D79L+S87P+A112P+D142L;

T46R+D79L+S87P+T116V+D142L;

D79L+P81R+S87P+A112P+D142L;

A27K+D79L+S87P+A112P+T124V+D142L;

D79L+Y82F+S87P+A112P+T124V+D142L;

D79L+Y82F+S87P+A112P+T124V+D142L;

D79L+S87P+A112P+T124V+A126V+D142L;

D79L+S87P+A112P+D142L;

D79L+Y82F+S87P+A112P+D142L;

S38T+D79L+S87P+A112P+A126V+D142L;

D79L+Y82F+S87P+A112P+A126V+D142L;

A27K+D79L+S87P+A112P+A126V+D142L;

D79L+S87P+N98C+A112P+G135C+D142L;

D79L+S87P+A112P+D142L+T141C+M161C;

S36P+D79L+S87P+A112P+D142L;

A37P+D79L+S87P+A112P+D142L;

S49P+D79L+S87P+A112P+D142L;

S50P+D79L+S87P+A112P+D142L;

D79L+S87P+D104P+A112P+D142L;

D79L+Y82F+S87G+A112P+D142L;

S70V+D79L+Y82F+S87G+Y97W+A112P+D142L;

D79L+Y82F+S87G+Y97W+D104P+A112P+D142L;

S70V+D79L+Y82F+S87G+A112P+D142L;

D79L+Y82F+S87G+D104P+A112P+D142L;

D79L+Y82F+S87G+A112P+A126V+D142L;

Y82F+S87G+S70V+D79L+D104P+A112P+D142L;

Y82F+S87G+D79L+D104P+A112P+A126V+D142L;

A27K+D79L+Y82F+S87G+D104P+A112P+A126V+D142L;

A27K+Y82F+S87G+D104P+A112P+A126V+D142L;

A27K+D79L+Y82F+D104P+A112P+A126V+D142L;

A27K+Y82F+D104P+A112P+A126V+D142L;

A27K+D79L+S87P+A112P+D142L; and

D79L+S87P+D142L.

In one embodiment, the thermostable protease is a variant of the metallo protease disclosed as the mature part of SEQ ID NO: 2 disclosed in WO 2003/048353 or the mature part of SEQ ID NO: 1 in WO 2010/008841 (SEQ ID NO: 292 herein) with one of the following substitutions or combinations of substitutions:

D79L+S87P+A112P+D142L;

D79L+S87P+D142L; and

A27K+D79L+Y82F+S87G+D104P+A112P+A126V+D142L.

In one embodiment, the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 (SEQ ID NO: 292 herein).

The thermostable protease may also be derived from any bacterium as long as the protease has the thermostability properties.

In one embodiment, the thermostable protease is derived from a strain of the bacterium Pyrococcus, such as a strain of Pyrococcus furiosus (pfu protease), for example, the Pyrococcus furiosus protease of SEQ ID NO: 291 or a variant thereof having at least 80% identity, such as at least 85%, such as at least 90%, such as at least 95%, such as at least 96%, such as at least 97%, such as at least 98%, such as at least 99% identity thereto.

In one embodiment, the protease is one shown as SEQ ID NO: 1 of U.S. Pat. No. 6,358,726-B1 (Takara Shuzo Company).

In one embodiment, the thermostable protease is a protease having at least 80% identity, such as at least 85%, such as at least 90%, such as at least 95%, such as at least 96%, such as at least 97%, such as at least 98%, such as at least 99% identity to SEQ ID NO: 1 of U.S. Pat. No. 6,358,726-B1. The Pyroccus furiosus protease can be purchased from Takara Bio, Japan.

The Pyrococcus furiosus protease is a thermostable protease. The commercial product Pyrococcus furiosus protease (PfuS) was found to have a thermostability of 110% (80° C./70° C.) and 103% (90° C./70° C.) at pH 4.5.

In one embodiment, a thermostable protease used in a process described herein has a thermostability value of more than 20% determined as Relative Activity at 80° C./70° C.

In one embodiment, the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In one embodiment, protease has a thermostability of between 20 and 50%, such as between 20 and 40%, such as 20 and 30% determined as Relative Activity at 80° C./70° C. In one embodiment, the protease has a thermostability between 50 and 115%, such as between 50 and 70%, such as between 50 and 60%, such as between 100 and 120%, such as between 105 and 115% determined as Relative Activity at 80° C./70° C.

In one embodiment, the protease has a thermostability value of more than 10% determined as Relative Activity at 85° C./70° C.

In one embodiment, the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In one embodiment, the protease has a thermostability of between 10% and 50%, such as between 10% and 30%, such as between 10% and 25% determined as Relative Activity at 85° C./70° C.

In one embodiment, the protease has more than 20%, more than 30%, more than 40%, more than 50%, more than 60%, more than 70%, more than 80%, more than 90% determined as Remaining Activity at 80° C.; and/or the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in the art (e.g., WO2018/098381).

In one embodiment, the protease may have a themostability for above 90, such as above 100 at 85° C. as determined using a Zein-BCA assay.

In one embodiment, the protease has a themostability above 60%, such as above 90%, such as above 100%, such as above 110% at 85° C. as determined using a Zein-BCA assay.

In one embodiment, protease has a themostability between 60-120, such as between 70-120%, such as between 80-120%, such as between 90-120%, such as between 100-120%, such as 110-120% at 85° C. as determined using a Zein-BCA assay.

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by a AZCL-casein assay.

Additional proteases contemplated for use with the present invention can be found in WO2018/222990 (the content of which is incorporated herein).

Additional polynucleotides encoding suitable proteases may be obtained from microorganisms of any genus, including those readily available within the UniProtKB database (www.uniprot.org).

The protease coding sequences can also be used to design nucleic acid probes to identify and clone DNA encoding proteases from strains of different genera or species, as described supra.

The polynucleotides encoding proteases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding proteases are described supra.

The protease can also include fused polypeptides or cleavable fusion polypeptides, as described supra.

In one embodiment, the thermostable protease is a serine protease, e.g., an S8 protease, such as one disclosed in PCT/US2018/054212, filed on Oct. 3, 2018, which is hereby incorporated herein by reference in its entirety.

In an embodiment, the S8 protease is derived from Palaeococcus, for instance Palaeococcus ferrophilus, such as the Palaeococcus ferrophilus S8 protease of SEQ ID NO: 293, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 293.

In an embodiment, the S8 protease is derived from Thermococcus, for instance Thermococcus litoralis or Thermococcus thioreducens, such as the Thermococcus litoralis S8 protease of SEQ ID NO: 294, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 294, or the Thermococcus thioreducens S8 protease of SEQ ID NO: 295, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 295.

### Glucoamylase in Liquefaction

A glucoamylase may optionally be present and/or added in liquefaction step step and/or the slurry prior to optional jet cook and/or liquefaction. In one embodiment, the glucoamylase is added together with or separately from the alpha-amylase and/or the optional protease, endoglucanase, phospholipase C, xylanase, phytase, and/or pullulanase.

In some embodiments, the fermenting organism comprises a heterologous polynucleotide encoding a glucoamylase, for example, as described in WO2017/087330, the content of which is hereby incorporated by reference. Any glucoamylase described or referenced herein is contemplated for expression in the fermenting organism.

The glucoamylase may be any glucoamylase that is suitable for the host cells and/or the methods described herein, such as a naturally occurring glucoamylase or a variant thereof that retains glucoamylase activity. The Glucoamylase in liquefcation may be any glucoamylase described in this section and/or any glucoamylase described in “Glucoamylase in Saccharification and/or Fermentation” described below.

In some embodiments, the fermenting organism comprising a heterologous polynucleotide encoding an glucoamylase has an increased level of glucoamylase activity compared to the host cells without the heterologous polynucleotide encoding the glucoamylase, when cultivated under the same conditions. In some embodiments, the fermenting organism has an increased level of glucoamylase activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the glucoamylase, when cultivated under the same conditions.

Exemplary glucoamylases that can be used with the host cells and/or the methods described herein include bacterial, yeast, or filamentous fungal glucoamylases, e.g., obtained from any of the microorganisms described or referenced herein, as described supra.

In one embodiment, the glucoamylase has a Relative Activity heat stability at 85° C. of at least 20%, at least 30%, or at least 35% determined as described in Example 4 of WO2018/098381 (heat stability).

In one embodiment, the glucoamylase has a relative activity pH optimum at pH 5.0 of at least 90%, e.g., at least 95%, at least 97%, or 100% determined as described in Example 4 of WO2018/098381 (pH optimum).

In one embodiment, the glucoamylase has a pH stability at pH 5.0 of at least 80%, at least 85%, at least 90% determined as described in Example 4 of WO2018/098381 (pH stability).

In one embodiment, the glucoamylase, such as a Penicillium oxalicum glucoamylase variant, used in liquefaction has a thermostability determined as DSC Td at pH 4.0 as described in Example 15 of WO2018/098381 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C. In one embodiment, the glucoamylase, such as a Penicillium oxalicum glucoamylase variant has a thermostability determined as DSC Td at pH 4.0 as described in Example 15 of WO2018/098381 in the range between 70° C. and 95° C., such as between 80° C. and 90° C.

In one embodiment, the glucoamylase, such as a Penicillium oxalicum glucoamylase variant, used in liquefaction has a thermostability determined as DSC Td at pH 4.8 as described in Example 15 of WO2018/098381 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C., such as at least 91° C. In one embodiment, the glucoamylase, such as a Penicillium oxalicum glucoamylase variant has a thermostability determined as DSC Td at pH 4.8 as described in Example 15 of WO2018/098381 in the range between 70° C. and 95° C., such as between 80° C. and 90° C.

In one embodiment, the glucoamylase, such as a Penicillium oxalicum glucoamylase variant, used in liquefaction has a residual activity determined as described in Example 16 of WO2018/098381, of at least 100% such as at least 105%, such as at least 110%, such as at least 115%, such as at least 120%, such as at least 125%. In one embodiment, the glucoamylase, such as a Penicillium oxalicum glucoamylase variant has a thermostability determined as residual activity as described in Example 16 of WO2018/098381, in the range between 100% and 130%.

In one embodiment, the glucoamylase, e.g., of fungal origin such as a filamentous fungi, from a strain of the genus Penicillium, e.g., a strain of Penicillium oxalicum, in particular the Penicillium oxalicum glucoamylase disclosed as SEQ ID NO: 2 in WO2011/127802 (which is hereby incorporated by reference) and shown in SEQ ID NO: 9 or 14 herein.

In one embodiment, the glucoamylase has at least 80%, e.g., at least 85%, at least 90%, at least 91%, at least 92%, at least 93%, at least 94%, at least 95%, at least 96%, at least 97%, at least 98%, at least 99% or 100% identity to the mature polypeptide shown in SEQ ID NO: 2 in WO2011/127802.

In one embodiment, the glucoamylase is a variant of the Penicillium oxalicum glucoamylase disclosed as SEQ ID NO: 2 in WO2011/127802 and shown in SEQ ID NO: 9 and 14 herein, having a K79V substitution (using the mature sequence shown in SEQ ID NO: 14 herein for numbering). The K79V glucoamylase variant has reduced sensitivity to protease degradation relative to the parent as disclosed in WO2013/036526 (which is hereby incorporated by reference).

In one embodiment, the glucoamylase is derived from Penicillium oxalicum.

In one embodiment, the glucoamylase is a variant of the Penicillium oxalicum glucoamylase disclosed as SEQ ID NO: 2 in WO2011/127802. In one embodiment, the Penicillium oxalicum glucoamylase is the one disclosed as SEQ ID NO: 2 in WO2011/127802 having Val (V) in position 79.

Contemplated Penicillium oxalicum glucoamylase variants are disclosed in WO2013/053801 which is hereby incorporated by reference.

In one embodiment, these variants have reduced sensitivity to protease degradation.

In one embodiment, these variant have improved thermostability compared to the parent.

In one embodiment, the glucoamylase has a K79V substitution (using SEQ ID NO: 2 of WO2011/127802 for numbering), corresponding to the PE001 variant, and further comprises one of the following alterations or combinations of alterations

T65A; Q327F; E501V; Y504T; Y504*; T65A+Q327F; T65A+E501V; T65A+Y504T; T65A+Y504*; Q327F+E501V; Q327F+Y504T; Q327F+Y504*; E501V+Y504T; E501V+Y504*; T65A+Q327F+E501V; T65A+Q327F+Y504T; T65A+E501V+Y504T; Q327F+E501V+Y504T; T65A+Q327F+Y504*; T65A+E501V+Y504*; Q327F+E501V+Y504*; T65A+Q327F+E501V+Y504T; T65A+Q327F+E501V+Y504*; E501V+Y504T; T65A+K161S; T65A+Q405T; T65A+Q327W; T65A+Q327F; T65A+Q327Y; P11F+T65A+Q327F; R1K+D3W+K5Q+G7V+N8S+T10K+P11S+T65A+Q327F; P2N+P4S+P11F+T65A+Q327F; P11F+D26C+K33C+T65A+Q327F; P2N+P4S+P11F+T65A+Q327W+E501V+Y504T; R1E+D3N+P4G+G6R+G7A+N8A+T10D+P11D+T65A+Q327F; P11F+T65A+Q327W; P2N+P4S+P11F+T65A+Q327F+E501V+Y504T; P11F+T65A+Q327W+E501V+Y504T; T65A+Q327F+E501V+Y504T; T65A+S105P+Q327W; T65A+S105P+Q327F; T65A+Q327W+S364P; T65A+Q327F+S364P; T65A+S103N+Q327F; P2N+P4S+P11F+K34Y+T65A+Q327F; P2N+P4S+P11F+T65A+Q327F+D445N+V447S; P2N+P4S+P11F+T65A+I172V+Q327F; P2N+P4S+P11F+T65A+Q327F+N502*; P2N+P4S+P11F+T65A+Q327F+N502T+P563S+K571E; P2N+P4S+P11F+R31S+K33V+T65A+Q327F+N564D+K571S; P2N+P4S+P11F+T65A+Q327F+S377T; P2N+P4S+P11F+T65A+V325T+Q327W; P2N+P4S+P11F+T65A+Q327F+D445N+V447S+E501V+Y504T; P2N+P4S+P11F+T65A+I172V+Q327F+E501V+Y504T; P2N+P4S+P11F+T65A+Q327F+S377T+E501V+Y504T; P2N+P4S+P11F+D26N+K34Y+T65A+Q327F; P2N+P4S+P11F+T65A+Q327F+I375A+E501V+Y504T; P2N+P4S+P11F+T65A+K218A+K221D+Q327F+E501V+Y504T; P2N+P4S+P11F+T65A+S103N+Q327F+E501V+Y504T; P2N+P4S+T10D+T65A+Q327F+E501V+Y504T; P2N+P4S+F12Y+T65A+Q327F+E501V+Y504T; K5A+P11F+T65A+Q327F+E501V+Y504T; P2N+P4S+T10E+E18N+T65A+Q327F+E501V+Y504T; P2N+T10E+E18N+T65A+Q327F+E501V+Y504T; P2N+P4S+P11F+T65A+Q327F+E501V+Y504T+T568N; P2N+P4S+P11F+T65A+Q327F+E501V+Y504T+K524T+G526A; P2N+P4S+P11F+K34Y+T65A+Q327F+D445N+V447S+E501V+Y504T; P2N+P4S+P11F+R31S+K33V+T65A+Q327F+D445N+V447S+E501V+Y504T; P2N+P4S+P11F+D26N+K34Y+T65A+Q327F+E501V+Y504T; P2N+P4S+P11F+T65A+F80*+Q327F+E501V+Y504T; P2N+P4S+P11F+T65A+K112S+Q327F+E501V+Y504T; P2N+P4S+P11F+T65A+Q327F+E501V+Y504T+T516P+K524T+G526A; P2N+P4S+P11F+T65A+Q327F+E501V+N502T+Y504*; P2N+P4S+P11F+T65A+Q327F+E501V+Y504T; P2N+P4S+P11F+T65A+S103N+Q327F+E501V+Y504T; K5A+P11F+T65A+Q327F+E501V+Y504T; P2N+P4S+P11F+T65A+Q327F+E501V+Y504T+T516P+K524T+G526A; P2N+P4S+P11F+T65A+V79A+Q327F+E501V+Y504T; P2N+P4S+P11F+T65A+V79G+Q327F+E501V+Y504T; P2N+P4S+P11F+T65A+V791+Q327F+E501V+Y504T; P2N+P4S+P11F+T65A+V79L+Q327F+E501V+Y504T; P2N+P4S+P11F+T65A+V79S+Q327F+E501V+Y504T; P2N+P4S+P11F+T65A+L72V+Q327F+E501V+Y504T; S255N+Q327F+E501V+Y504T; P2N+P4S+P11F+T65A+E74N+V79K+Q327F+E501V+Y504T; P2N+P4S+P11F+T65A+G220N+Q327F+E501V+Y504T; P2N+P4S+P11F+T65A+Y245N+Q327F+E501V+Y504T; P2N+P4S+P11F+T65A+Q253N+Q327F+E501V+Y504T; P2N+P4S+P11F+T65A+D279N+Q327F+E501V+Y504T; P2N+P4S+P11F+T65A+Q327F+S359N+E501V+Y504T; P2N+P4S+P11F+T65A+Q327F+D370N+E501V+Y504T; P2N+P4S+P11F+T65A+Q327F+V460S+E501V+Y504T; P2N+P4S+P11F+T65A+Q327F+V460T+P468T+E501V+Y504T; P2N+P4S+P11F+T65A+Q327F+T463N+E501V+Y504T; P2N+P4S+P11F+T65A+Q327F+S465N+E501V+Y504T; and P2N+P4S+P11F+T65A+Q327F+T477N+E501V+Y504T.

In one embodiment, the Penicillium oxalicum glucoamylase variant has a K79V substitution (using SEQ ID NO: 2 of WO2011/127802 for numbering), corresponding to the PE001 variant, and further comprises one of the following substitutions or combinations of substitutions:

P11F+T65A+Q327F;

P2N+P4S+P11F+T65A+Q327F;

P11F+D26C+K330+T65A+Q327F;

P2N+P4S+P11F+T65A+Q327W+E501V+Y504T;

P2N+P4S+P11F+T65A+Q327F+E501V+Y504T; and

P11F+T65A+Q327W+E501V+Y504T.

The glucoamylase may be added in amounts from 0.1-100 micrograms EP/g, such as 0.5-50 micrograms EP/g, such as 1-25 micrograms EP/g, such as 2-12 micrograms EP/g DS.

Additional polynucleotides encoding suitable glucoamylases may be obtained from microorganisms of any genus, including those readily available within the UniProtKB database (www.uniprot.org).

The glucoamylase coding sequences can also be used to design nucleic acid probes to identify and clone DNA encoding glucoamylases from strains of different genera or species, as described supra.

The polynucleotides encoding glucoamylases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding glucoamylases are described supra.

In one embodiment, the glucoamylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any glucoamylase described or referenced herein. In one aspect, the glucoamylase sequence differs by no more than ten amino acids, e.g., by no more than five amino acids, by no more than four amino acids, by no more than three amino acids, by no more than two amino acids, or by one amino acid from any glucoamylase described or referenced herein. In one embodiment, the glucoamylase comprises or consists of the amino acid sequence of any glucoamylase described or referenced herein, allelic variant, or a fragment thereof having glucoamylase activity. In one embodiment, the glucoamylase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the glucoamylase has at least 20%, e.g., at least 40%, at least 50%, at least 60%, at least 70%, at least 80%, at least 90%, at least 95%, at least 96%, at least 97%, at least 98%, at least 99%, or 100% of the glucoamylase activity of any glucoamylase described or referenced herein under the same conditions.

In one embodiment, the glucoamylase coding sequence hybridizes under at least low stringency conditions, e.g., medium stringency conditions, medium-high stringency conditions, high stringency conditions, or very high stringency conditions with the full-length complementary strand of the coding sequence from any glucoamylase described or referenced herein. In one embodiment, the glucoamylase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any glucoamylase described or referenced herein.

In one embodiment, the polynucleotide encoding the glucoamylase comprises the coding sequence of any glucoamylase described or referenced herein. In one embodiment, the polynucleotide encoding the glucoamylase comprises a subsequence of the coding sequence from any glucoamylase described or referenced herein, wherein the subsequence encodes a polypeptide having glucoamylase activity. In one embodiment, the number of nucleotides residues in the subsequence is at least 75%, e.g., at least 80%, 85%, 90%, or 95% of the number of the referenced coding sequence.

The glucoamylase can also include fused polypeptides or cleavable fusion polypeptides, as described supra.

### Pullulanases

In some embodiments, a pullulanase is present and/or added added in the slurry prior to optional jet cook and/or liquefaction, in the liquefaction step and/or saccharification step, or simultaneous saccharification and fermentation (SSF).

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

In some embodiments, the fermenting organism comprises a heterologous polynucleotide encoding a pullulanase. Any pullulanase described or referenced herein is contemplated for expression in the fermenting organism.

The pullulanase may be any pullulanase that is suitable for the host cells and/or the methods described herein, such as a naturally occurring pullulanase or a variant thereof that retains pullulanase activity.

In some embodiments, the fermenting organism comprising a heterologous polynucleotide encoding a pullulanase has an increased level of pullulanase activity compared to the host cells without the heterologous polynucleotide encoding the pullulanase, when cultivated under the same conditions. In some embodiments, the fermenting organism has an increased level of pullulanase activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the pullulanase, when cultivated under the same conditions.

Exemplary pullulanasees that can be used with the host cells and/or the methods described herein include bacterial, yeast, or filamentous fungal pullulanases, e.g., obtained from any of the microorganisms described or referenced herein, as described supra.

Contemplated pullulanases include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated include the pullulanases from Pyrococcus woesei, specifically from Pyrococcus woesei DSM No. 3773 disclosed in WO92/02614.

In one embodiment, the pullulanase is a family GH57 pullulanase. In one embodiment, the pullulanase includes an X47 domain as disclosed in U.S. Pat. No. 61/289,040 published as WO 2011/087836 (which are hereby incorporated by reference). More specifically the pullulanase may be derived from a strain of the genus Thermococcus, including Thermococcus litoralis and Thermococcus hydrothermalis, such as the Thermococcus hydrothermalis pullulanase truncated at site X4 right after the X47 domain (i.e., amino acids 1-782). The pullulanase may also be a hybrid of the Thermococcus litoralis and Thermococcus hydrothermalis pullulanases or a T. hydrothermalis/T. litoralis hybrid enzyme with truncation site X4 disclosed in U.S. Ser. No. 61/289,040 published as WO 2011/087836 (which is hereby incorporated by reference).

In another embodiment, the pullulanase is one comprising an X46 domain disclosed in WO 2011/076123 (Novozymes).

The pullulanase may be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in WO2018/098381.

Suitable commercially available pullulanase products include PROMOZYME D, PROMOZYME™ D2 (Novozymes A/S, Denmark), OPTIMAX L-300 (DuPont-Danisco, USA), and AMANO 8 (Amano, Japan).

In one embodiment, the pullulanase is derived from the Bacillus subtilis pullulanase of SEQ ID NO: 114 disclosed in PCT/US2018/054212, filed Oct. 3, 2018. In one embodiment, the pullulanase is derived from the Bacillus licheniformis pullulanase of SEQ ID NO: 115 disclosed in PCT/US2018/054212. In one embodiment, the pullulanase is derived from the Oryza sativa pullulanase of SEQ ID NO: 116 disclosed in PCT/US2018/054212. In one embodiment, the pullulanase is derived from the Triticum aestivum pullulanase of SEQ ID NO: 117 disclosed in PCT/US2018/054212. In one embodiment, the pullulanase is derived from the Clostridium phytofermentans pullulanase of SEQ ID NO: 118 disclosed in PCT/US2018/054212. In one embodiment, the pullulanase is derived from the Streptomyces avermitilis pullulanase of SEQ ID NO: 119 disclosed in PCT/US2018/054212. In one embodiment, the pullulanase is derived from the Klebsiella pneumoniae pullulanase of SEQ ID NO: 120 disclosed in PCT/US2018/054212.

Additional pullulanases contemplated for use with the present invention can be found in WO2011/153516 (the content of which is incorporated herein).

Additional polynucleotides encoding suitable pullulanases may be obtained from microorganisms of any genus, including those readily available within the UniProtKB database (www.uniprot.org).

The pullulanase coding sequences can also be used to design nucleic acid probes to identify and clone DNA encoding pullulanases from strains of different genera or species, as described supra.

The polynucleotides encoding pullulanases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding pullulanases are described supra.

In one embodiment, the pullulanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any pullulanase described or referenced herein. In one aspect, the pullulanase sequence differs by no more than ten amino acids, e.g., by no more than five amino acids, by no more than four amino acids, by no more than three amino acids, by no more than two amino acids, or by one amino acid from any pullulanase described or referenced herein. In one embodiment, the pullulanase comprises or consists of the amino acid sequence of any pullulanase described or referenced herein, allelic variant, or a fragment thereof having pullulanase activity. In one embodiment, the pullulanase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the pullulanase has at least 20%, e.g., at least 40%, at least 50%, at least 60%, at least 70%, at least 80%, at least 90%, at least 95%, at least 96%, at least 97%, at least 98%, at least 99%, or 100% of the pullulanase activity of any pullulanase described or referenced herein under the same conditions.

In one embodiment, the pullulanase coding sequence hybridizes under at least low stringency conditions, e.g., medium stringency conditions, medium-high stringency conditions, high stringency conditions, or very high stringency conditions with the full-length complementary strand of the coding sequence from any pullulanase described or referenced herein. In one embodiment, the pullulanase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any pullulanase described or referenced herein.

In one embodiment, the polynucleotide encoding the pullulanase comprises the coding sequence of any pullulanase described or referenced herein. In one embodiment, the polynucleotide encoding the pullulanase comprises a subsequence of the coding sequence from any pullulanase described or referenced herein, wherein the subsequence encodes a polypeptide having pullulanase activity. In one embodiment, the number of nucleotides residues in the subsequence is at least 75%, e.g., at least 80%, 85%, 90%, or 95% of the number of the referenced coding sequence.

The pullulanase can also include fused polypeptides or cleavable fusion polypeptides, as described supra.

### Saccharification and Fermentation of Starch-Containing Material

In aspects using a starch-containing material, a glucoamylase may be present and/or added in saccharification step a) and/or fermentation step b) or simultaneous saccharification and fermentation (SSF). The glucoamylase of the saccharification step a) and/or fermentation step b) or simultaneous saccharification and fermentation (SSF) is typically different from the glucoamylase optionally added to any liquefaction step described supra. In one embodiment, the glucoamylase is present and/or added together with a fungal alpha-amylase.

In some aspects, the fermenting organism comprises a heterologous polynucleotide encoding a glucoamylase, for example, as described in WO 2017/087330, the content of which is hereby incorporated by reference.

Examples of glucoamylases can be found in the “Glucoamylases in Saccharification and/or Fermentation” section below.

When doing sequential saccharification and fermentation, saccharification step a) may be carried out under conditions well-known in the art. For instance, saccharification step a) may last up to from about 24 to about 72 hours. In one embodiment, pre-saccharification is done. Pre-saccharification is typically done for 40-90 minutes at a temperature between 30-65° C., typically about 60° C. Pre-saccharification is, in one embodiment, followed by saccharification during fermentation in simultaneous saccharification and fermentation (SSF). Saccharification is typically carried out at temperatures from 20-75° C., preferably from 40-70° C., typically about 60° C., and typically at a pH between 4 and 5, such as about pH 4.5.

Fermentation is carried out in a fermentation medium, as known in the art and, e.g., as described herein. The fermentation medium includes the fermentation substrate, that is, the carbohydrate source that is metabolized by the fermenting organism. With the processes described herein, the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

In some embodiments, the fermenting organism provides (or is capable of providing) an ethanol yield increase over Saccharomyces cerevisiae strain Ethanol Red® (deposited under Accession No. V14/007039 at National Measurement Institute, Victoria, Australia) of more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, using the same process set-up and conditions, e.g., conditions described herein. Improved ethanol yields can be measures at about or after 10, 20, 30, 40 50, 60 or 70 hours fermentation.

In some embodiments, the fermenting organism provides (or is capable of providing) an ethanol yield increase of more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, when compared to an otherwise identical fermenting organism lacking the genetic modification that increases or decreases expression of a regulator, when using the same process set-up and conditions, e.g., conditions described herein. Improved ethanol yields can be measures at about or after 10, 20, 30, 40 50, 60 or 70 hours fermentation.

Generally, fermenting organisms such as yeast, including Saccharomyces cerevisiae yeast, require an adequate source of nitrogen for propagation and fermentation. Many sources of supplemental nitrogen, if necessary, can be used and such sources of nitrogen are well known in the art. The nitrogen source may be organic, such as urea, DDGs, wet cake or corn mash, or inorganic, such as ammonia or ammonium hydroxide. In one embodiment, the nitrogen source is urea.

In some embodiments, the fermenting organism requires less supplemental nitrogen (e.g., urea, ammonia, ammonium hydroxide) during fermentation to maintain the same or greater yield of fermentation product (e.g., ethanol), when compared to an otherwise identical fermenting organism lacking the genetic modification that increases or decreases expression of a transporter, or regulator thereof. In some embodiments, the fermenting organism requires less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, supplemental nitrogen (e.g., urea, ammonia, ammonium hydroxide) during fermentation to maintain the same yield of fermentation product (e.g., ethanol), when compared to an otherwise identical fermenting organism lacking the genetic modification that increases or decreases expression of a transporter, or regulator thereof. In some embodiments, the fermenting organism requires no supplemental nitrogen (e.g., urea, ammonia, ammonium hydroxide) during fermentation to maintain the same yield of fermentation product (e.g., ethanol), when compared to an otherwise identical fermenting organism lacking the genetic modification that increases or decreases expression of a transporter, or regulator thereof. Fermentaion product yields can be measures at about or after 10, 20, 30, 40 50, 60 or 70 hours fermentation.

In some embodiments, the fermenting organism efficiently utilizes tripeptides and/or tetrapeptides in the fermentation medium, thereby decreasing the residual concentration following fermentation. Methods of determining amount (e.g., concentration) of tripeptides and tetrapeptides in the fermentation medium are known in the art and described in the examples herein. In some embodiments, the fermenting organism decreases (or is capable of decreasing) the amount of residual tripeptides and/or tetrapeptides in the fermentation medium after 29 hours of fermentation (e.g., under conditions described herein), by at least 5%, e.g., 10%, 15%, 20%, 25%, 30%, 35%, 40%, 45%, 50%, 55%, 60%, 65%, 70%, 75%, 80%, 85%, 90%, or 95%, when compared to an otherwise identical fermenting organism lacking the genetic modification that increases or decreases expression of a transporter, or regulator thereof.

Simultaneous saccharification and fermentation (“SSF”) is widely used in industrial scale fermentation product production processes, especially ethanol production processes. When doing SSF the saccharification step a) and the fermentation step b) are carried out simultaneously. There is no holding stage for the saccharification, meaning that a fermenting organism, such as yeast, and enzyme(s), may be added together. However, it is also contemplated to add the fermenting organism and enzyme(s) separately. SSF is typically carried out at a temperature from 25° C. to 40° C., such as from 28° C. to 35° C., such as from 30° C. to 34° C., or about 32° C. In one embodiment, fermentation is ongoing for 6 to 120 hours, in particular 24 to 96 hours. In one embodiment, the pH is between 4-5.

In one embodiment, a cellulolytic enzyme composition is present and/or added in saccharification, fermentation or simultaneous saccharification and fermentation (SSF). Examples of such cellulolytic enzyme compositions can be found in the “Cellulolytic Enzyme Composition” section below. The cellulolytic enzyme composition may be present and/or added together with a glucoamylase, such as one disclosed in the “Glucoamylase in Saccharification and/or Fermentation” section below.

### Glucoamylase in Saccharification and/or Fermentation

Glucoamylase may be present and/or added in saccharification, fermentation or simultaneous saccharification and fermentation (SSF).

As described supra, in some embodiments, the fermenting organism comprises a heterologous polynucleotide encoding an glucoamylase, for example, as described in WO2017/087330, the content of which is hereby incorporated by reference. Any glucoamylase described or referenced herein is contemplated for expression in the fermenting organism.

The glucoamylase may be any glucoamylase that is suitable for the host cells and/or the methods described herein, such as a naturally occurring glucoamylase or a variant thereof that retains glucoamylase activity.

In some embodiments, the fermenting organism comprising a heterologous polynucleotide encoding a glucoamylase has an increased level of glucoamylase activity compared to the host cells without the heterologous polynucleotide encoding the glucoamylase, when cultivated under the same conditions. In some embodiments, the fermenting organism has an increased level of glucoamylase activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the glucoamylase, when cultivated under the same conditions.

Exemplary glucoamylases that can be used with the host cells and/or the methods described herein include bacterial, yeast, or filamentous fungal glucoamylases, e.g., obtained from any of the microorganisms described or referenced herein, as described supra.

The glucoamylase may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). In one embodiment, the glucoamylase used during saccharification and/or fermentation is the Talaromyces emersonii glucoamylase disclosed in WO 99/28448.

Bacterial glucoamylases contemplated include glucoamylases from the genus Clostridium, in particular C. thermoamylolyticum (EP 135,138), and C. thermohydrosulfuricum (WO 86/01831).

Contemplated fungal glucoamylases include Trametes cingulate, Pachykytospora papyracea; and Leucopaxillus giganteus all disclosed in WO 2006/069289; or Peniophora rufomarginata disclosed in WO2007/124285; or a mixture thereof. Also hybrid glucoamylase are contemplated. Examples include the hybrid glucoamylases disclosed in WO2005/045018.

In one embodiment, the glucoamylase is derived from a strain of the genus Pycnoporus, in particular a strain of Pycnoporus as described in WO2011/066576 (SEQ ID NO: 2, 4 or 6 therein), including the Pycnoporus sanguineus glucoamylase, or from a strain of the genus Gloeophyllum, such as a strain of Gloeophyllum sepiarium or Gloeophyllum trabeum, in particular a strain of Gloeophyllum as described in WO2011/068803 (SEQ ID NO: 2, 4, 6, 8, 10, 12, 14 or 16 therein). In one embodiment, the glucoamylase is SEQ ID NO: 2 in WO2011/068803 (i.e. Gloeophyllum sepiarium glucoamylase).

In one embodiment, the glucoamylase is a Gloeophyllum trabeum glucoamylase (disclosed as SEQ ID NO: 3 in WO2014/177546). In another embodiment, the glucoamylase is derived from a strain of the genus Nigrofomes, in particular a strain of Nigrofomes sp. disclosed in WO2012/064351 (SEQ ID NO: 2 therein).

Also contemplated are glucoamylases which exhibit a high identity to any of the above mentioned glucoamylases, i.e., at least 60%, such as at least 70%, at least 75%, at least 80%, at least 85%, at least 90%, at least 95%, at least 96%, at least 97%, at least 98%, at least 99% or even 100% identity to any one of the mature enzyme sequences mentioned above.

Glucoamylases may be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Glucoamylases may be added to the saccharification and/or fermentation in an amount of 1-1,000 μg EP/g DS, preferably 10-500 μg/gDS, especially between 25-250 μg/g DS.

In one embodiment, the glucoamylase is added as a blend further comprising an alpha-amylase. In one embodiment, the alpha-amylase is a fungal alpha-amylase, especially an acid fungal alpha-amylase. The alpha-amylase is typically a side activity.

In one embodiment, the glucoamylase is a blend comprising Talaromyces emersonii glucoamylase disclosed in WO 99/28448 as SEQ ID NO: 34 and Trametes cingulata glucoamylase disclosed as SEQ ID NO: 2 in WO 06/069289.

In one embodiment, the glucoamylase is a blend comprising Talaromyces emersonii glucoamylase disclosed in WO 99/28448 (SEQ ID NO: 19 herein), Trametes cingulata glucoamylase disclosed as SEQ ID NO: 2 in WO 06/69289, and an alpha-amylase.

In one embodiment, the glucoamylase is a blend comprising Talaromyces emersonii glucoamylase disclosed in WO99/28448, Trametes cingulata glucoamylase disclosed in WO 06/69289, and Rhizomucor pusillus alpha-amylase with Aspergillus niger glucoamylase linker and SBD disclosed as V039 in Table 5 in WO2006/069290.

In one embodiment, the glucoamylase is a blend comprising Gloeophyllum sepiarium glucoamylase shown as SEQ ID NO: 2 in WO2011/068803 and an alpha-amylase, in particular Rhizomucor pusillus alpha-amylase with an Aspergillus niger glucoamylase linker and starch-binding domain (SBD), disclosed SEQ ID NO: 3 in WO2013/006756, in particular with the following substitutions: G128D+D143N.

In one embodiment, the alpha-amylase may be derived from a strain of the genus Rhizomucor, preferably a strain the Rhizomucor pusillus, such as the one shown in SEQ ID NO: 3 in WO2013/006756, or the genus Meripilus, preferably a strain of Meripilus giganteus. In one embodiment, the alpha-amylase is derived from a Rhizomucor pusillus with an Aspergillus niger glucoamylase linker and starch-binding domain (SBD), disclosed as V039 in Table 5 in WO2006/069290.

In one embodiment, the Rhizomucor pusillus alpha-amylase or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; and G128D+Y141W+D143N+K192R; or G128D+Y141W+D143N+K192R+P219C (using SEQ ID NO: 3 in WO 2013/006756 for numbering).

In one embodiment, the glucoamylase blend comprises Gloeophyllum sepiarium glucoamylase (e.g., SEQ ID NO: 2 in WO2011/068803) and Rhizomucor pusillus alpha-amylase.

In one embodiment, the glucoamylase blend comprises Gloeophyllum sepiarium glucoamylase shown as SEQ ID NO: 2 in WO2011/068803 and Rhizomucor pusillus with an Aspergillus niger glucoamylase linker and starch-binding domain (SBD), disclosed SEQ ID NO: 3 in WO2013/006756 with the following substitutions: G128D+D143N.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE™, and AMG™ E (from Novozymes A/S); OPTIDEX™ 300, GC480, GC417 (from DuPont-Danisco); AMIGASE™ and AMIGASE™ PLUS (from DSM); G-ZYME™ G900, G-ZYME™ and G990 ZR (from DuPont-Danisco).

In one embodiment, the glucoamylase is derived from the Debaryomyces occidentalis glucoamylase shown as SEQ ID NO: 102 in WO2018/222990. In one embodiment, the glucoamylase is derived from the Saccharomycopsis fibuligera glucoamylase shown as SEQ ID NO: 103 of WO2018/222990. In one embodiment, the glucoamylase is derived from the Saccharomycopsis fibuligera glucoamylase shown as SEQ ID NO: 104 of WO2018/222990. In one embodiment, the glucoamylase is derived from the Saccharomyces cerevisiae glucoamylase shown as SEQ ID NO: 105 of WO2018/222990. In one embodiment, the glucoamylase is derived from the Aspergillus niger glucoamylase shown as SEQ ID NO: 106 of WO2018/222990. In one embodiment, the glucoamylase is derived from the Aspergillus oryzae glucoamylase shown as SEQ ID NO: 107 of WO2018/222990. In one embodiment, the glucoamylase is derived from the Rhizopus oryzae glucoamylase shown as SEQ ID NO: 108 of WO2018/222990. In one embodiment, the glucoamylase is derived from the Clostridium thermocellum glucoamylase shown as SEQ ID NO: 109 of WO2018/222990. In one embodiment, the glucoamylase is derived from the Clostridium thermocellum glucoamylase shown as SEQ ID NO: 110 of WO2018/222990. In one embodiment, the glucoamylase is derived from the Arxula adeninivorans glucoamylase shown as SEQ ID NO: 111 of WO2018/222990. In one embodiment, the glucoamylase is derived from the Hormoconis resinae glucoamylase shown as SEQ ID NO: 112 of WO2018/222990. In one embodiment, the glucoamylase is derived from the Aureobasidium pullulans glucoamylase shown as SEQ ID NO: 113 of WO2018/222990.

Additional glucoamylases contemplated for use with the present invention can be found in WO2011/153516 (the content of which is incorporated herein).

Additional polynucleotides encoding suitable glucoamylases may be obtained from microorganisms of any genus, including those readily available within the UniProtKB database (www.uniprot.org).

The glucoamylase coding sequences can also be used to design nucleic acid probes to identify and clone DNA encoding glucoamylases from strains of different genera or species, as described supra.

The polynucleotides encoding glucoamylases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding glucoamylases are described supra.

In one embodiment, the glucoamylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any glucoamylase described or referenced herein (e.g., the Saccharomycopsis fibuligera glucoamylase shown as SEQ ID NO: 103 or 104 of WO2018/222990). In one aspect, the glucoamylase sequence differs by no more than ten amino acids, e.g., by no more than five amino acids, by no more than four amino acids, by no more than three amino acids, by no more than two amino acids, or by one amino acid from any glucoamylase described or referenced herein (e.g., the Saccharomycopsis fibuligera glucoamylase shown as SEQ ID NO: 103 or 104 of WO2018/222990). In one embodiment, the glucoamylase comprises or consists of the amino acid sequence of any glucoamylase described or referenced herein (e.g., the Saccharomycopsis fibuligera glucoamylase shown as SEQ ID NO: 103 or 104 of WO2018/222990), allelic variant, or a fragment thereof having glucoamylase activity. In one embodiment, the glucoamylase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the glucoamylase has at least 20%, e.g., at least 40%, at least 50%, at least 60%, at least 70%, at least 80%, at least 90%, at least 95%, at least 96%, at least 97%, at least 98%, at least 99%, or 100% of the glucoamylase activity of any glucoamylase described or referenced herein (e.g., the Saccharomycopsis fibuligera glucoamylase shown as SEQ ID NO: 103 or 104 of WO2018/222990) under the same conditions.

In one embodiment, the glucoamylase coding sequence hybridizes under at least low stringency conditions, e.g., medium stringency conditions, medium-high stringency conditions, high stringency conditions, or very high stringency conditions with the full-length complementary strand of the coding sequence from any glucoamylase described or referenced herein (e.g., the Saccharomycopsis fibuligera glucoamylase shown as SEQ ID NO: 103 or 104 of WO2018/222990). In one embodiment, the glucoamylase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any glucoamylase described or referenced herein (e.g., the Saccharomycopsis fibuligera glucoamylase shown as SEQ ID NO: 103 or 104 of WO2018/222990).

In one embodiment, the polynucleotide encoding the glucoamylase comprises the coding sequence of any glucoamylase described or referenced herein (e.g., the Saccharomycopsis fibuligera glucoamylase shown as SEQ ID NO: 103 or 104 of WO2018/222990). In one embodiment, the polynucleotide encoding the glucoamylase comprises a subsequence of the coding sequence from any glucoamylase described or referenced herein, wherein the subsequence encodes a polypeptide having glucoamylase activity. In one embodiment, the number of nucleotides residues in the subsequence is at least 75%, e.g., at least 80%, 85%, 90%, or 95% of the number of the referenced coding sequence.

The glucoamylase can also include fused polypeptides or cleavable fusion polypeptides, as described supra.

### Methods Using a Cellulosic-Containing Material

In some aspects, the methods described herein produce a fermentation product from a cellulosic-containing material. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic-containing material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one embodiment, the cellulosic-containing material is any biomass material. In another embodiment, the cellulosic-containing material is lignocellulose, which comprises cellulose, hemicelluloses, and lignin.

In one embodiment, the cellulosic-containing material is agricultural residue, herbaceous material (including energy crops), municipal solid waste, pulp and paper mill residue, waste paper, or wood (including forestry residue).

In another embodiment, the cellulosic-containing material is arundo, bagasse, bamboo, corn cob, corn fiber, corn stover, miscanthus, rice straw, switchgrass, or wheat straw.

In another embodiment, the cellulosic-containing material is aspen, eucalyptus, fir, pine, poplar, spruce, or willow.

In another embodiment, the cellulosic-containing material is algal cellulose, bacterial cellulose, cotton linter, filter paper, microcrystalline cellulose (e.g., AVICEL®), or phosphoric-acid treated cellulose.

In another embodiment, the cellulosic-containing material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic-containing material may be used as is or may be subjected to pretreatment, using conventional methods known in the art, as described herein. In a preferred embodiment, the cellulosic-containing material is pretreated.

The methods of using cellulosic-containing material can be accomplished using methods conventional in the art. Moreover, the methods of can be implemented using any conventional biomass processing apparatus configured to carry out the processes.

### Cellulosic Pretreatment

In one embodiment the cellulosic-containing material is pretreated before saccharification in step (a).

In practicing the processes described herein, any pretreatment process known in the art can be used to disrupt plant cell wall components of the cellulosic-containing material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic-containing material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In a one embodiment, the cellulosic-containing material is pretreated before saccharification (i.e., hydrolysis) and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

In one embodiment, the cellulosic-containing material is pretreated with steam. In steam pretreatment, the cellulosic-containing material is heated to disrupt the plant cell wall components, including lignin, hemicellulose, and cellulose to make the cellulose and other fractions, e.g., hemicellulose, accessible to enzymes. The cellulosic-containing material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic-containing material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. patent application Ser. No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

In one embodiment, the cellulosic-containing material is subjected to a chemical pretreatment. The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic-containing material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115). In a specific embodiment the dilute acid pretreatment of cellulosic-containing material is carried out using 4% w/w sulfuric acid at 180° C. for 5 minutes.

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment. Lime pretreatment is performed with calcium oxide or calcium hydroxide at temperatures of 85-150° C. and residence times from one hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO2006/032282).

Ammonia fiber expansion (AFEX) involves treating the cellulosic-containing material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic-containing material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one embodiment, the chemical pretreatment is carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the cellulosic-containing material and held at a temperature in the range of preferably 140-200° C., e.g., 165-190° C., for periods ranging from 1 to 60 minutes.

In another embodiment, pretreatment takes place in an aqueous slurry. In preferred aspects, the cellulosic-containing material is present during pretreatment in amounts preferably between 10-80 wt. %, e.g., 20-70 wt. % or 30-60 wt. %, such as around 40 wt. %. The pretreated cellulosic-containing material can be unwashed or washed using any method known in the art, e.g., washed with water.

In one embodiment, the cellulosic-containing material is subjected to mechanical or physical pretreatment. The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic-containing material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in one embodiment, the cellulosic-containing material is subjected to physical (mechanical) or chemical pretreatment, or any combination thereof, to promote the separation and/or release of cellulose, hemicellulose, and/or lignin.

In one embodiment, the cellulosic-containing material is subjected to a biological pretreatment. The term “biological pretreatment” refers to any biological pretreatment that promotes the separation and/or release of cellulose, hemicellulose, and/or lignin from the cellulosic-containing material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, DC, 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, DC, chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Saccharification and Fermentation of Cellulosic-Containing Material

Saccharification (i.e., hydrolysis) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF).

SHF uses separate process steps to first enzymatically hydrolyze the cellulosic-containing material to fermentable sugars, e.g., glucose, cellobiose, and pentose monomers, and then ferment the fermentable sugars to ethanol. In SSF, the enzymatic hydrolysis of the cellulosic-containing material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, DC, 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation organismcan tolerate. It is understood herein that any method known in the art comprising pretreatment, enzymatic hydrolysis (saccharification), fermentation, or a combination thereof, can be used in the practicing the processes described herein.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

In the saccharification step (i.e., hydrolysis step), the cellulosic and/or starch-containing material, e.g., pretreated, is hydrolyzed to break down cellulose, hemicellulose, and/or starch to fermentable sugars, such as glucose, cellobiose, xylose, xylulose, arabinose, mannose, galactose, and/or soluble oligosaccharides. The hydrolysis is performed enzymatically e.g., by a cellulolytic enzyme composition. The enzymes of the compositions can be added simultaneously or sequentially.

Enzymatic hydrolysis may be carried out in a suitable aqueous environment under conditions that can be readily determined by one skilled in the art. In one aspect, hydrolysis is performed under conditions suitable for the activity of the enzymes(s), i.e., optimal for the enzyme(s). The hydrolysis can be carried out as a fed batch or continuous process where the cellulosic and/or starch-containing material is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 120 hours, e.g., about 16 to about 72 hours or about 24 to about 48 hours. The temperature is in the range of preferably about 25° C. to about 70° C., e.g., about 30° C. to about 65° C., about 40° C. to about 60° C., or about 50° C. to about 55° C. The pH is in the range of preferably about 3 to about 8, e.g., about 3.5 to about 7, about 4 to about 6, or about 4.5 to about 5.5. The dry solids content is in the range of preferably about 5 to about 50 wt. %, e.g., about 10 to about 40 wt. % or about 20 to about 30 wt. %.

Saccharification in step (a) may be carried out using a cellulolytic enzyme composition. Such enzyme compositions are described below in the “Cellulolytic Enzyme Composition” section below. The cellulolytic enzyme compositions can comprise any protein useful in degrading the cellulosic-containing material. In one aspect, the cellulolytic enzyme composition comprises or further comprises one or more (e.g., two, several) proteins selected from the group consisting of a cellulase, an AA9 (GH61) polypeptide, a hemicellulase, an esterase, an expansin, a ligninolytic enzyme, an oxidoreductase, a pectinase, a protease, and a swollenin.

In another embodiment, the cellulase is preferably one or more (e.g., two, several) enzymes selected from the group consisting of an endoglucanase, a cellobiohydrolase, and a beta-glucosidase.

In another embodiment, the hemicellulase is preferably one or more (e.g., two, several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another embodiment, the oxidoreductase is one or more (e.g., two, several) enzymes selected from the group consisting of a catalase, a laccase, and a peroxidase.

The enzymes or enzyme compositions used in a processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

In one embodiment, an effective amount of cellulolytic or hemicellulolytic enzyme composition to the cellulosic-containing material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic-containing material.

In one embodiment, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide (GH61 polypeptide) can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one embodiment, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In the fermentation step, sugars, released from the cellulosic-containing material, e.g., as a result of the pretreatment and enzymatic hydrolysis steps, are fermented to ethanol, by a fermenting organism, such as yeast described herein. Hydrolysis (saccharification) and fermentation can be separate or simultaneous.

Any suitable hydrolyzed cellulosic-containing material can be used in the fermentation step in practicing the processes described herein. Such feedstocks include, but are not limited to carbohydrates (e.g., lignocellulose, xylans, cellulose, starch, etc.). The material is generally selected based on economics, i.e., costs per equivalent sugar potential, and recalcitrance to enzymatic conversion.

Production of ethanol by a fermenting organism using cellulosic-containing material results from the metabolism of sugars (monosaccharides). The sugar composition of the hydrolyzed cellulosic-containing material and the ability of the fermenting organism to utilize the different sugars has a direct impact in process yields.

Compositions of the fermentation media and fermentation conditions depend on the fermenting organism and can easily be determined by one skilled in the art. Typically, the fermentation takes place under conditions known to be suitable for generating the fermentation product. In some embodiments, the fermentation process is carried out under aerobic or microaerophilic (i.e., where the concentration of oxygen is less than that in air), or anaerobic conditions. In some embodiments, fermentation is conducted under anaerobic conditions (i.e., no detectable oxygen), or less than about 5, about 2.5, or about 1 mmol/L/h oxygen. In the absence of oxygen, the NADH produced in glycolysis cannot be oxidized by oxidative phosphorylation. Under anaerobic conditions, pyruvate or a derivative thereof may be utilized by the host cell as an electron and hydrogen acceptor in order to generate NAD+.

The fermentation process is typically run at a temperature that is optimal for the recombinant fungal cell. For example, in some embodiments, the fermentation process is performed at a temperature in the range of from about 25° C. to about 42° C. Typically the process is carried out a temperature that is less than about 38° C., less than about 35° C., less than about 33° C., or less than about 38° C., but at least about 20° C., 22° C., or 25° C.

A fermentation stimulator can be used in a process described herein to further improve the fermentation, and in particular, the performance of the fermenting organism, such as, rate enhancement and product yield (e.g., ethanol yield). A “fermentation stimulator” refers to stimulators for growth of the fermenting organisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

### Cellulolytic Enzymes and Compositions

A cellulolytic enzyme or cellulolytic enzyme composition may be present and/or added during saccharification in step (a). A cellulolytic enzyme composition is an enzyme preparation containing one or more (e.g., two, several) enzymes that hydrolyze cellulosic-containing material. Such enzymes include endoglucanase, cellobiohydrolase, beta-glucosidase, and/or combinations thereof.

In some embodiments, the fermenting organism comprises one or more (e.g., two, several) heterologous polynucleotides encoding enzymes that hydrolyze cellulosic-containing material (e.g., an endoglucanase, cellobiohydrolase, beta-glucosidase or combinations thereof). Any enzyme described or referenced herein that hydrolyzes cellulosic-containing material is contemplated for expression in the fermenting organism.

The cellulolytic enzyme may be any cellulolytic enzyme that is suitable for the host cells and/or the methods described herein (e.g., an endoglucanase, cellobiohydrolase, beta-glucosidase), such as a naturally occurring cellulolytic enzyme or a variant thereof that retains cellulolytic enzyme activity.

In some embodiments, the fermenting organism comprising a heterologous polynucleotide encoding a cellulolytic enzyme has an increased level of cellulolytic enzyme activity (e.g., increased endoglucanase, cellobiohydrolase, and/or beta-glucosidase) compared to the host cells without the heterologous polynucleotide encoding the cellulolytic enzyme, when cultivated under the same conditions. In some embodiments, the fermenting organism has an increased level of cellulolytic enzyme activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the cellulolytic enzyme, when cultivated under the same conditions.

Exemplary cellulolytic enzymes that can be used with the host cells and/or the methods described herein include bacterial, yeast, or filamentous fungal cellulolytic enzymes, e.g., obtained from any of the microorganisms described or referenced herein, as described supra.

The cellulolytic enzyme may be of any origin. In an embodiment the cellulolytic enzyme is derived from a strain of Trichoderma, such as a strain of Trichoderma reesei; a strain of Humicola, such as a strain of Humicola insolens, and/or a strain of Chrysosporium, such as a strain of Chrysosporium lucknowense. In a preferred embodiment the cellulolytic enzyme is derived from a strain of Trichoderma reesei.

The cellulolytic enzyme composition may further comprise one or more of the following polypeptides, such as enzymes: AA9 polypeptide (GH61 polypeptide) having cellulolytic enhancing activity, beta-glucosidase, xylanase, beta-xylosidase, CBH I, CBH II, or a mixture of two, three, four, five or six thereof.

The further polypeptide(s) (e.g., AA9 polypeptide) and/or enzyme(s) (e.g., beta-glucosidase, xylanase, beta-xylosidase, CBH I and/or CBH II may be foreign to the cellulolytic enzyme composition producing organism (e.g., Trichoderma reesei).

In an embodiment, the cellulolytic enzyme composition comprises an AA9 polypeptide having cellulolytic enhancing activity and a beta-glucosidase.

In another embodiment, the cellulolytic enzyme composition comprises an AA9 polypeptide having cellulolytic enhancing activity, a beta-glucosidase, and a CBH I.

In another embodiment, the cellulolytic enzyme composition comprises an AA9 polypeptide having cellulolytic enhancing activity, a beta-glucosidase, a CBH I and a CBH II.

Other enzymes, such as endoglucanases, may also be comprised in the cellulolytic enzyme composition.

As mentioned above, the cellulolytic enzyme composition may comprise a number of difference polypeptides, including enzymes.

In one embodiment, the cellulolytic enzyme composition is a Trichoderma reesei cellulolytic enzyme composition, further comprising Thermoascus aurantiacus AA9 (GH61A) polypeptide having cellulolytic enhancing activity (e.g., WO2005/074656), and Aspergillus oryzae beta-glucosidase fusion protein (e.g., one disclosed in WO2008/057637, in particular shown as SEQ ID NOs: 59 and 60 therein).

In another embodiment, the cellulolytic enzyme composition is a Trichoderma reesei cellulolytic enzyme composition, further comprising Thermoascus aurantiacus AA9 (GH61A) polypeptide having cellulolytic enhancing activity (e.g., SEQ ID NO: 2 in WO2005/074656), and Aspergillus fumigatus beta-glucosidase (e.g., SEQ ID NO: 2 of WO2005/047499).

In another embodiment, the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, and Aspergillus fumigatus beta-glucosidase (e.g., SEQ ID NO: 2 of WO2005/047499).

In another embodiment, the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, and Aspergillus fumigatus beta-glucosidase (e.g., SEQ ID NO: 2 of WO2005/047499) or a variant disclosed in WO 2012/044915 (hereby incorporated by reference), in particular one comprising one or more such as all of the following substitutions: F100D, S283G, N456E, F512Y.

In an embodiment, the cellulolytic enzyme composition is a Trichoderma reesei cellulolytic composition, further comprising an AA9 (GH61A) polypeptide having cellulolytic enhancing activity, in particular the one derived from a strain of Penicillium emersonii (e.g., SEQ ID NO: 2 in WO2011/041397), Aspergillus fumigatus beta-glucosidase (e.g., SEQ ID NO: 2 in WO2005/047499) variant with one or more, in particular all of the following substitutions: F100D, S283G, N456E, F512Y and disclosed in WO2012/044915; Aspergillus fumigatus Cel7A CBH1, e.g., the one disclosed as SEQ ID NO: 6 in WO2011/057140 and Aspergillus fumigatus CBH II, e.g., the one disclosed as SEQ ID NO: 18 in WO2011/057140.

In one embodiment, the cellulolytic enzyme composition is a Trichoderma reesei, cellulolytic enzyme composition, further comprising a hemicellulase or hemicellulolytic enzyme composition, such as an Aspergillus fumigatus xylanase and Aspergillus fumigatus beta-xylosidase.

In one embodiment, the cellulolytic enzyme composition also comprises a xylanase (e.g., derived from a strain of the genus Aspergillus, in particular Aspergillus aculeatus or Aspergillus fumigatus; or a strain of the genus Talaromyces, in particular Talaromyces leycettanus) and/or a beta-xylosidase (e.g., derived from Aspergillus, in particular Aspergillus fumigatus, or a strain of Talaromyces, in particular Talaromyces emersonii).

In an embodiment, the cellulolytic enzyme composition is a Trichoderma reesei cellulolytic enzyme composition, further comprising Thermoascus aurantiacus AA9 (GH61A) polypeptide having cellulolytic enhancing activity (e.g., WO2005/074656), Aspergillus oryzae beta-glucosidase fusion protein (e.g., one disclosed in WO2008/057637, in particular as SEQ ID NOs: 59 and 60), and Aspergillus aculeatus xylanase (e.g., Xyl II in WO 94/21785).

In another embodiment, the cellulolytic enzyme composition comprises a Trichoderma reesei cellulolytic preparation, further comprising Thermoascus aurantiacus GH61A polypeptide having cellulolytic enhancing activity (e.g., SEQ ID NO: 2 in WO2005/074656), Aspergillus fumigatus beta-glucosidase (e.g., SEQ ID NO: 2 of WO2005/047499) and Aspergillus aculeatus xylanase (Xyl II disclosed in WO 94/21785).

In another embodiment, the cellulolytic enzyme composition comprises a Trichoderma reesei cellulolytic enzyme composition, further comprising Thermoascus aurantiacus AA9 (GH61A) polypeptide having cellulolytic enhancing activity (e.g., SEQ ID NO: 2 in WO2005/074656), Aspergillus fumigatus beta-glucosidase (e.g., SEQ ID NO: 2 of WO2005/047499) and Aspergillus aculeatus xylanase (e.g., Xyl II disclosed in WO 94/21785).

In another embodiment, the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499) and Aspergillus fumigatus xylanase (e.g., Xyl III in WO2006/078256).

In another embodiment, the cellulolytic enzyme composition comprises a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499), Aspergillus fumigatus xylanase (e.g., Xyl III in WO2006/078256), and CBH I from Aspergillus fumigatus, in particular Cel7A CBH1 disclosed as SEQ ID NO: 2 in WO2011/057140.

In another embodiment, the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499), Aspergillus fumigatus xylanase (e.g., Xyl III in WO2006/078256), CBH I from Aspergillus fumigatus, in particular Cel7A CBH1 disclosed as SEQ ID NO: 2 in WO2011/057140, and CBH II derived from Aspergillus fumigatus in particular the one disclosed as SEQ ID NO: 4 in WO2013/028928.

In another embodiment, the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO 2005/047499) or variant thereof with one or more, in particular all, of the following substitutions: F100D, S283G, N456E, F512Y; Aspergillus fumigatus xylanase (e.g., Xyl III in WO2006/078256), CBH I from Aspergillus fumigatus, in particular Cel7A CBH I disclosed as SEQ ID NO: 2 in WO2011/057140, and CBH II derived from Aspergillus fumigatus, in particular the one disclosed in WO2013/028928.

In another embodiment, the cellulolytic enzyme composition is a Trichoderma reesei cellulolytic enzyme composition comprising the CBH I (GENSEQP Accession No. AZY49536 (WO2012/103293); a CBH II (GENSEQP Accession No. AZY49446 (WO2012/103288); a beta-glucosidase variant (GENSEQP Accession No. AZU67153 (WO2012/44915)), in particular with one or more, in particular all, of the following substitutions: F100D, S283G, N456E, F512Y; and AA9 (GH61 polypeptide) (GENSEQP Accession No. BAL61510 (WO2013/028912)).

In another embodiment, the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288); a GH10 xylanase (GENSEQP Accession No. BAK46118 (WO2013/019827)); and a beta-xylosidase (GENSEQP Accession No. AZ104896 (WO2011/057140)).

In another embodiment, the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)); and an AA9 (GH61 polypeptide; GENSEQP Accession No. BAL61510 (WO2013/028912)).

In another embodiment, the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)), an AA9 (GH61 polypeptide; GENSEQP Accession No. BAL61510 (WO2013/028912)), and a catalase (GENSEQP Accession No. BAC11005 (WO2012/130120)).

In an embodiment, the cellulolytic enzyme composition is a Trichoderma reesei cellulolytic enzyme composition comprising a CBH I (GENSEQP Accession No. AZY49446 (WO2012/103288); a CBH II (GENSEQP Accession No. AZY49446 (WO2012/103288)), a beta-glucosidase variant (GENSEQP Accession No. AZU67153 (WO2012/44915)), with one or more, in particular all, of the following substitutions: F100D, S283G, N456E, F512Y; an AA9 (GH61 polypeptide; GENSEQP Accession No. BAL61510 (WO2013/028912)), a GH10 xylanase (GENSEQP Accession No. BAK46118 (WO2013/019827)), and a beta-xylosidase (GENSEQP Accession No. AZ104896 (WO2011/057140)).

In an embodiment, the cellulolytic composition is a Trichoderma reesei cellulolytic enzyme preparation comprising an EG I (Swissprot Accession No. P07981), EG II (EMBL Accession No. M19373), CBH I (supra); CBH II (supra); beta-glucosidase variant (supra) with the following substitutions: F100D, S283G, N456E, F512Y; an AA9 (GH61 polypeptide; supra), GH10 xylanase (supra); and beta-xylosidase (supra).

All cellulolytic enzyme compositions disclosed in WO2013/028928 are also contemplated and hereby incorporated by reference.

The cellulolytic enzyme composition comprises or may further comprise one or more (several) proteins selected from the group consisting of a cellulase, a AA9 (i.e., GH61) polypeptide having cellulolytic enhancing activity, a hemicellulase, an expansin, an esterase, a laccase, a ligninolytic enzyme, a pectinase, a peroxidase, a protease, and a swollenin.

In one embodiment, the cellulolytic enzyme composition is a commercial cellulolytic enzyme composition. Examples of commercial cellulolytic enzyme compositions suitable for use in a process of the invention include: CELLIC® CTec (Novozymes A/S), CELLIC® CTec2 (Novozymes A/S), CELLIC® CTec3 (Novozymes A/S), CELLUCLAST™ (Novozymes A/S), SPEZYME™ CP (Genencor Int.), ACCELLERASE™ 1000, ACCELLERASE 1500, ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme composition may be added in an amount effective from about 0.001 to about 5.0 wt. % of solids, e.g., about 0.025 to about 4.0 wt. % of solids or about 0.005 to about 2.0 wt. % of solids.

Additional enzymes, and compositions thereof can be found in WO2011/153516 and WO2016/045569 (the contents of which are incorporated herein).

Additional polynucleotides encoding suitable cellulolytic enzymes may be obtained from microorganisms of any genus, including those readily available within the UniProtKB database (www.uniprot.org).

The cellulolytic enzyme coding sequences can also be used to design nucleic acid probes to identify and clone DNA encoding cellulolytic enzymes from strains of different genera or species, as described supra.

The polynucleotides encoding cellulolytic enzymes may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding cellulolytic enzymes are described supra.

In one embodiment, the cellulolytic enzyme has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any cellulolytic enzyme described or referenced herein (e.g., any endoglucanase, cellobiohydrolase, or beta-glucosidase). In one aspect, the cellulolytic enzyme sequence differs by no more than ten amino acids, e.g., by no more than five amino acids, by no more than four amino acids, by no more than three amino acids, by no more than two amino acids, or by one amino acid from any cellulolytic enzyme described or referenced herein. In one embodiment, the cellulolytic enzyme comprises or consists of the amino acid sequence of any cellulolytic enzyme described or referenced herein, allelic variant, or a fragment thereof having cellulolytic enzyme activity. In one embodiment, the cellulolytic enzyme has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the cellulolytic enzyme has at least 20%, e.g., at least 40%, at least 50%, at least 60%, at least 70%, at least 80%, at least 90%, at least 95%, at least 96%, at least 97%, at least 98%, at least 99%, or 100% of the cellulolytic enzyme activity of any cellulolytic enzyme described or referenced herein (e.g., any endoglucanase, cellobiohydrolase, or beta-glucosidase) under the same conditions.

In one embodiment, the cellulolytic enzyme coding sequence hybridizes under at least low stringency conditions, e.g., medium stringency conditions, medium-high stringency conditions, high stringency conditions, or very high stringency conditions with the full-length complementary strand of the coding sequence from any cellulolytic enzyme described or referenced herein (e.g., any endoglucanase, cellobiohydrolase, or beta-glucosidase). In one embodiment, the cellulolytic enzyme coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any cellulolytic enzyme described or referenced herein.

In one embodiment, the polynucleotide encoding the cellulolytic enzyme comprises the coding sequence of any cellulolytic enzyme described or referenced herein (e.g., any endoglucanase, cellobiohydrolase, or beta-glucosidase). In one embodiment, the polynucleotide encoding the cellulolytic enzyme comprises a subsequence of the coding sequence from any cellulolytic enzyme described or referenced herein, wherein the subsequence encodes a polypeptide having cellulolytic enzyme activity. In one embodiment, the number of nucleotides residues in the subsequence is at least 75%, e.g., at least 80%, 85%, 90%, or 95% of the number of the referenced coding sequence.

The cellulolytic enzyme can also include fused polypeptides or cleavable fusion polypeptides, as described supra.

### Xylose Metabolism

In one aspect, the fermenting organism (e.g., yeast cell) further comprises a heterologous polynucleotide encoding a xylose isomerase (XI). The xylose isomerase may be any xylose isomerase that is suitable for the host cells and the methods described herein, such as a naturally occurring xylose isomerase or a variant thereof that retains xylose isomerase activity. In one embodiment, the xylose isomerase is present in the cytosol of the host cells.

In some embodiments, the fermenting organism comprising a heterologous polynucleotide encoding a xylose isomerase has an increased level of xylose isomerase activity compared to the host cells without the heterologous polynucleotide encoding the xylose isomerase, when cultivated under the same conditions. In some embodiments, the fermenting organisms have an increased level of xylose isomerase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylose isomerase, when cultivated under the same conditions.

Exemplary xylose isomerases that can be used with the recombinant host cells and methods of use described herein include, but are not limited to, XIs from the fungus Piromyces sp. (WO2003/062430) or other sources (Madhavan et al., 2009, Appl Microbiol Biotechnol. 82(6), 1067-1078) have been expressed in S. cerevisiae host cells. Still other XIs suitable for expression in yeast have been described in US 2012/0184020 (an XI from Ruminococcus flavefaciens), WO2011/078262 (several XIs from Reticulitermes speratus and Mastotermes darwiniensis) and WO2012/009272 (constructs and fungal cells containing an XI from Abiotrophia defectiva). U.S. Pat. No. 8,586,336 describes a S. cerevisiae host cell expressing an XI obtained by bovine rumen fluid.

Additional polynucleotides encoding suitable xylose isomerases may be obtained from microorganisms of any genus, including those readily available within the UniProtKB database (www.uniprot.org). In one embodiment, the xylose isomerases is a bacterial, a yeast, or a filamentous fungal xylose isomerase, e.g., obtained from any of the microorganisms described or referenced herein, as described supra.

The xylose isomerase coding sequences can also be used to design nucleic acid probes to identify and clone DNA encoding xylose isomerases from strains of different genera or species, as described supra.

The polynucleotides encoding xylose isomerases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding xylose isomerases are described supra.

In one embodiment, the xylose isomerase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylose isomerase described or referenced herein. In one aspect, the xylose isomerase sequence differs by no more than ten amino acids, e.g., by no more than five amino acids, by no more than four amino acids, by no more than three amino acids, by no more than two amino acids, or by one amino acid from any xylose isomerase described or referenced herein. In one embodiment, the xylose isomerase comprises or consists of the amino acid sequence of any xylose isomerase described or referenced herein, allelic variant, or a fragment thereof having xylose isomerase activity. In one embodiment, the xylose isomerase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylose isomerase has at least 20%, e.g., at least 40%, at least 50%, at least 60%, at least 70%, at least 80%, at least 90%, at least 95%, at least 96%, at least 97%, at least 98%, at least 99%, or 100% of the xylose isomerase activity of any xylose isomerase described or referenced herein under the same conditions.

In one embodiment, the xylose isomerase coding sequence hybridizes under at least low stringency conditions, e.g., medium stringency conditions, medium-high stringency conditions, high stringency conditions, or very high stringency conditions with the full-length complementary strand of the coding sequence from any xylose isomerase described or referenced herein. In one embodiment, the xylose isomerase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylose isomerase described or referenced herein.

In one embodiment, the heterologous polynucleotide encoding the xylose isomerase comprises the coding sequence of any xylose isomerase described or referenced herein. In one embodiment, the heterologous polynucleotide encoding the xylose isomerase comprises a subsequence of the coding sequence from any xylose isomerase described or referenced herein, wherein the subsequence encodes a polypeptide having xylose isomerase activity. In one embodiment, the number of nucleotides residues in the subsequence is at least 75%, e.g., at least 80%, 85%, 90%, or 95% of the number of the referenced coding sequence.

The xylose isomerases can also include fused polypeptides or cleavable fusion polypeptides, as described supra.

In one aspect, the fermenting organism (e.g., yeast cell) further comprises a heterologous polynucleotide encoding a xylulokinase (XK). A xylulokinase, as used herein, provides enzymatic activity for converting D-xylulose to xylulose 5-phosphate. The xylulokinase may be any xylulokinase that is suitable for the host cells and the methods described herein, such as a naturally occurring xylulokinase or a variant thereof that retains xylulokinase activity. In one embodiment, the xylulokinase is present in the cytosol of the host cells.

In some embodiments, the fermenting organisms comprising a heterologous polynucleotide encoding a xylulokinase have an increased level of xylulokinase activity compared to the host cells without the heterologous polynucleotide encoding the xylulokinase, when cultivated under the same conditions. In some embodiments, the host cells have an increased level of xylose isomerase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylulokinase, when cultivated under the same conditions.

Exemplary xylulokinases that can be used with the fermenting organisms and methods of use described herein include, but are not limited to, a Saccharomyces cerevisiae xylulokinase (e.g., the xylulokinase described as SEQ ID NO: 75 of WO2018/222990). Additional polynucleotides encoding suitable xylulokinases may be obtained from microorganisms of any genus, including those readily available within the UniProtKB database (www.uniprot.org). In one embodiment, the xylulokinases is a bacterial, a yeast, or a filamentous fungal xylulokinase, e.g., obtained from any of the microorganisms described or referenced herein, as described supra.

The xylulokinase coding sequences can also be used to design nucleic acid probes to identify and clone DNA encoding xylulokinases from strains of different genera or species, as described supra.

The polynucleotides encoding xylulokinases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding xylulokinases are described supra.

In one embodiment, the xylulokinase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylulokinase described or referenced herein. In one embodiment, the xylulokinase sequence differs by no more than ten amino acids, e.g., by no more than five amino acids, by no more than four amino acids, by no more than three amino acids, by no more than two amino acids, or by one amino acid from any xylulokinase described or referenced herein. In one embodiment, the xylulokinase comprises or consists of the amino acid sequence of any xylulokinase described or referenced herein, allelic variant, or a fragment thereof having xylulokinase activity. In one embodiment, the xylulokinase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylulokinase has at least 20%, e.g., at least 40%, at least 50%, at least 60%, at least 70%, at least 80%, at least 90%, at least 95%, at least 96%, at least 97%, at least 98%, at least 99%, or 100% of the xylulokinase activity of any xylulokinase described or referenced herein under the same conditions.

In one embodiment, the xylulokinase coding sequence hybridizes under at least low stringency conditions, e.g., medium stringency conditions, medium-high stringency conditions, high stringency conditions, or very high stringency conditions with the full-length complementary strand of the coding sequence from any xylulokinase described or referenced herein. In one embodiment, the xylulokinase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylulokinase described or referenced herein.

In one embodiment, the heterologous polynucleotide encoding the xylulokinase comprises the coding sequence of any xylulokinase described or referenced herein. In one embodiment, the heterologous polynucleotide encoding the xylulokinase comprises a subsequence of the coding sequence from any xylulokinase described or referenced herein, wherein the subsequence encodes a polypeptide having xylulokinase activity. In one embodiment, the number of nucleotides residues in the subsequence is at least 75%, e.g., at least 80%, 85%, 90%, or 95% of the number of the referenced coding sequence.

The xylulokinases can also include fused polypeptides or cleavable fusion polypeptides, as described supra.

In one aspect, the fermenting organism (e.g., yeast cell) further comprises a heterologous polynucleotide encoding a ribulose 5 phosphate 3-epimerase (RPE1). A ribulose 5 phosphate 3-epimerase, as used herein, provides enzymatic activity for converting L-ribulose 5-phosphate to L-xylulose 5-phosphate (EC 5.1.3.22). The RPE1 may be any RPE1 that is suitable for the host cells and the methods described herein, such as a naturally occurring RPE1 or a variant thereof that retains RPE1 activity. In one embodiment, the RPE1 is present in the cytosol of the host cells.

In one embodiment, the recombinant cell comprises a heterologous polynucleotide encoding a ribulose 5 phosphate 3-epimerase (RPE1), wherein the RPE1 is Saccharomyces cerevisiae RPE1, or an RPE1 having at least 60%, e.g., at least 65%, 70%, 75%, 80%, 85%, 90%, 95%, 97%, 98%, 99%, or 100% sequence identity to a Saccharomyces cerevisiae RPE1.

In one aspect, the fermenting organism (e.g., yeast cell) further comprises a heterologous polynucleotide encoding a ribulose 5 phosphate isomerase (RKI1). A ribulose 5 phosphate isomerase, as used herein, provides enzymatic activity for converting ribose-5-phophate to ribulose 5-phosphate. The RKI1 may be any RKI1 that is suitable for the host cells and the methods described herein, such as a naturally occurring RKI1 or a variant thereof that retains RKI1 activity. In one embodiment, the RKI1 is present in the cytosol of the host cells.

In one embodiment, the fermenting organism comprises a heterologous polynucleotide encoding a ribulose 5 phosphate isomerase (RKI1), wherein the RKI1 is a Saccharomyces cerevisiae RKI1, or an RKI1 having at least 60%, e.g., at least 65%, 70%, 75%, 80%, 85%, 90%, 95%, 97%, 98%, 99%, or 100% sequence identity to a Saccharomyces cerevisiae RKI1.

In one aspect, the fermenting organism (e.g., yeast cell) further comprises a heterologous polynucleotide encoding a transketolase (TKL1). The TKL1 may be any TKL1 that is suitable for the host cells and the methods described herein, such as a naturally occurring TKL1 or a variant thereof that retains TKL1 activity. In one embodiment, the TKL1 is present in the cytosol of the host cells.

In one embodiment, the fermenting organism comprises a heterologous polynucleotide encoding a transketolase (TKL1), wherein the TKL1 is a Saccharomyces cerevisiae TKL1, or a TKL1 having at least 60%, e.g., at least 65%, 70%, 75%, 80%, 85%, 90%, 95%, 97%, 98%, 99%, or 100% sequence identity to a Saccharomyces cerevisiae TKL1.

In one aspect, the fermenting organism (e.g., yeast cell) further comprises a heterologous polynucleotide encoding a transaldolase (TAL1). The TAL1 may be any TAL1 that is suitable for the host cells and the methods described herein, such as a naturally occurring TAL1 or a variant thereof that retains TAL1 activity. In one embodiment, the TAL1 is present in the cytosol of the host cells.

In one embodiment, the fermenting organism comprises a heterologous polynucleotide encoding a transketolase (TAL1), wherein the TAL1 is a Saccharomyces cerevisiae TAL1, or a TAL1 having at least 60%, e.g., at least 65%, 70%, 75%, 80%, 85%, 90%, 95%, 97%, 98%, 99%, or 100% sequence identity to a Saccharomyces cerevisiae TAL1.

### Fermentation Products

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one embodiment, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603. In one embodiment, the fermentation product is ethanol.

In another embodiment, the fermentation product is an alkane. The alkane may be an unbranched or a branched alkane. The alkane can be, but is not limited to, pentane, hexane, heptane, octane, nonane, decane, undecane, or dodecane.

In another embodiment, the fermentation product is a cycloalkane. The cycloalkane can be, but is not limited to, cyclopentane, cyclohexane, cycloheptane, or cyclooctane. In another aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another embodiment, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another embodiment, the fermentation product is isoprene.

In another embodiment, the fermentation product is a ketone. The term “ketone” encompasses a substance that contains one or more ketone moieties. The ketone can be, but is not limited to, acetone.

In another embodiment, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another embodiment, the fermentation product is polyketide.

### Recovery

The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

In some embodiments of the methods, the fermentation product after being recovered is substantially pure. With respect to the methods herein, “substantially pure” intends a recovered preparation that contains no more than 15% impurity, wherein impurity intends compounds other than the fermentation product (e.g., ethanol). In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose or xylose) can be quantified by HPLC using, for example, a refractive index detector for glucose and alcohols, and a UV detector for organic acids (Lin et al., Biotechnol. Bioeng. 90:775 -779 (2005)), or using other suitable assay and detection methods well known in the art.

The invention may further be described in the following numbered paragraphs:

Paragraph [1]. A method of producing a fermentation product from a starch-containing or cellulosic-containing material comprising:

(a) saccharifying the starch-containing or cellulosic-containing material; and
(b) fermenting the saccharified material of step (a) with a fermenting organism;

wherein the fermenting organism comprises a genetic modification that increases or decreases expression of a transporter, or regulator thereof.

Paragraph [2]. The method of paragraph [1], wherein the fermenting organism has increased or decreased expression (e.g., by at least 5%, e.g., at least 10%, at least 15%, at least 20%, at least 25%, at least 50%, at least 100%, at least 150%, at least 200%, at least 300%, or at 500%) of a transporter, or regulator thereof when compared to Saccharomyces cerevisiae strain Ethanol Red® (deposited under Accession No. V14/007039 at National Measurement Institute, Victoria, Australia) under the same conditions.

Paragraph [3]. The method of paragraph [1] or [2], wherein the fermenting organism requires less supplemental nitrogen (e.g., urea, ammonia, ammonium hydroxide) during fermentation to maintain the same yield of fermentation product, when compared to an otherwise identical fermenting organism lacking the genetic modification that increases or decreases expression of a transporter, or regulator thereof.

Paragraph [4]. The method of any one of paragraphs [1]-[3], wherein the fermenting organism comprises a genetic modification that increases or decreases expression of a transporter, such as any one of the transporters shown in Table 1 (e.g., any one of SEQ ID NOs: 86-170).

Paragraph [5]. The method of any one of paragraphs [1]-[4], wherein the fermenting organism comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters shown in Table 1 (e.g., any one of SEQ ID NOs: 86-170).

Paragraph [6]. The method of any one of paragraphs [1]-[5], wherein the fermenting organism comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 163 or SEQ ID NO: 164.

Paragraph [7]. The method of any one of paragraphs [1]-[6], wherein the fermenting organism comprises a heterologous polynucleotide encoding a transporter that differs by no more than ten amino acids, e.g., by no more than five amino acids, by no more than four amino acids, by no more than three amino acids, by no more than two amino acids, or by one amino acid from any one of the transporters shown in Table 1 (e.g., any one of SEQ ID NOs: 86-170).

Paragraph [8]. The method of any one of paragraphs [1]-[7], wherein the fermenting organism comprises a heterologous polynucleotide encoding a transporter comprising or consisting of the amino acid sequence of any one of the transporters shown in Table 1 (e.g., any one of SEQ ID NOs: 86-170).

Paragraph [9]. The method of any one of paragraphs [1]-[8], wherein the fermenting organism comprises a disruption to an endogenous transporter gene, such as any one of the transporter genes shown in Table 1 (e.g., any one of SEQ ID NOs: 1-85).

Paragraph [10]. The method of paragraph [9], wherein the disrupted endogenous transporter gene is inactivated.

Paragraph [11]. The method of paragraph [9] or [10], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [12]. The method of any one of paragraphs [9]-[11], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [13]. The method of any one of paragraphs [1]-[12], wherein the fermenting organism comprises a genetic modification that increases or decreases expression of a regulator, such as any one of the regulators shown in Table 2 (e.g., any one of SEQ ID NOs:

231-290).

Paragraph [14]. The method of any one of paragraphs [1]-[13], wherein the fermenting organism comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [15]. The method of any one of paragraphs [1]-[14], wherein the fermenting organism comprises a heterologous polynucleotide encoding regulator, wherein the regulator differs by no more than ten amino acids, e.g., by no more than five amino acids, by no more than four amino acids, by no more than three amino acids, by no more than two amino acids, or by one amino acid from any one of the regulators shown in Table 2 (e.g., any one of SEQ ID NOs: 231-290).

Paragraph [16]. The method of any one of paragraphs [1]-[15], wherein the fermenting organism comprises a heterologous polynucleotide encoding a regulator, wherein the regulator has an amino acid sequence comprising or consisting of the amino acid sequence of any one of the regulators shown in Table 2 (e.g., any one of SEQ ID NOs: 231-290).

Paragraph [17]. The method of any one of paragraphs [1]-[16], wherein the fermenting organism comprises a disruption to an endogenous regulator gene, such as any one of the regulator genes shown in Table 2 (e.g., any one of SEQ ID NOs: 171-230).

Paragraph [18]. The method of paragraph [17], wherein the disrupted endogenous regulator gene is inactivated.

Paragraph [19]. The method of paragraph [17] or [18], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [20]. The method of any one of paragraphs [17]-[19], wherein the endogenous regulator gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [21]. The method of any one of paragraphs [1]-[20], comprising liquefying the starch-containing material at a temperature above the initial gelatinization temperature in the presence of an alpha-amylase prior to saccharification.

Paragraph [22]. The method of paragraph [21], comprising adding a protease in liquefaction.

Paragraph [23]. The method of paragraph [22], wherein the protease is a serine protease, e.g., an S8 protease.

Paragraph [24]. The method of paragraph [23], wherein the protease is a bacterial protease, particularly a protease derived form Pyrococcus, Palaeococcus, or Thermococcus, more particularly Pyrococcus furiosus, Palaeococcus ferrophilus, Thermococcus litoralis, Thermococcus thioreducens.

Paragraph [25]. The method of any one of paragraphs [21]-[24], wherein the protease is selected from the group consisting of SEQ ID NO: 291, SEQ ID NO: 292, SEQ ID NO: 293, SEQ ID NO: 294, and SEQ ID NO: 295, or a variant of any one of SEQ ID NO: 291, SEQ ID NO: 292, SEQ ID NO: 293, SEQ ID NO: 294, and SEQ ID NO: 295 having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity thereto.

Paragraph [26]. The method of any one of paragraphs [1]-[25], wherein fermentation and saccharification are performed simultaneously in a simultaneous saccharification and fermentation (SSF).

Paragraph [27]. The method of any one of paragraphs [1]-[26], wherein fermentation and saccharification are performed sequentially (SHF).

Paragraph [28]. The method of any one of paragraphs [1]-[27], comprising recovering the fermentation product from the from the fermentation.

Paragraph [29]. The method of paragraph [28], wherein recovering the fermentation product from the from the fermentation comprises distillation.

Paragraph [30]. The method of any one of paragraphs [1]-[29], wherein the fermentation product is ethanol.

Paragraph [31]. The method of paragraph [30], wherein the ethanol yield is more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, greater than Saccharomyces cerevisiae strain Ethanol Red® (deposited under Accession No. V14/007039 at National Measurement Institute, Victoria, Australia) under the same conditions (e.g., under conditions described herein, such as after 53 hours fermentation).

Paragraph [32]. The method of paragraph [30] or [31], wherein the ethanol yield is more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, greater when compared to an otherwise identical fermenting organism lacking the genetic modification that increases or decreases expression of a regulator under the same conditions (e.g., under conditions described herein, such as after 53 hours fermentation).

Paragraph [33]. The method of any one of paragraphs [1]-[32], wherein saccharification of step (a) occurs on a starch-containing material, and wherein the starch-containing material is either gelatinized or ungelatinized starch.

Paragraph [34]. The method of any one of paragraphs [1]-[33], wherein the fermenting organism comprises a heterologous polynucleotide encoding a glucoamylase.

Paragraph [35]. The method of paragraph [34], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase described herein).

Paragraph [36]. The method of any one of paragraphs [1]-[35], wherein the fermenting organism comprises a heterologous polynucleotide encoding an alpha-amylase.

Paragraph [37]. The method of paragraph [21] or [36], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [38]. The method of any one of paragraphs [1]-[37], wherein the fermenting organism comprises a heterologous polynucleotide encoding a protease.

Paragraph [39]. The method of paragraph [38], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having any one of SEQ ID NOs: 9-73 of WO2018/222990).

Paragraph [40]. The method of any one of paragraphs [1]-[39], wherein saccharification of step (a) occurs on a cellulosic-containing material, and wherein the cellulosic-containing material is pretreated.

Paragraph [41]. The method of paragraph [36], wherein the pretreatment is a dilute acid pretreatment.

Paragraph [42]. The method of any one of paragraphs [1]-[41], wherein saccharification occurs on a cellulosic-containing material, and wherein the enzyme composition comprises one or more enzymes selected from a cellulase, an AA9 polypeptide, a hemicellulase, a CIP, an esterase, an expansin, a ligninolytic enzyme, an oxidoreductase, a pectinase, a protease, and a swollenin.

Paragraph [43]. The method of paragraph [42], wherein the cellulase is one or more enzymes selected from an endoglucanase, a cellobiohydrolase, and a beta-glucosidase.

Paragraph [44]. The method of paragraph [42], wherein the hemicellulase is one or more enzymes selected a xylanase, an acetylxylan esterase, a feruloyl esterase, an arabinofuranosidase, a xylosidase, and a glucuronidase.

Paragraph [45]. The method of any one of paragraphs [1]-[44], wherein the fermenting organism is a Saccharomyces, Rhodotorula, Schizosaccharomyces, Kluyveromyces, Pichia, Issatchenkia, Hansenula, Rhodosporidium, Candida, Torulaspora, Zygosaccharomyces, Yarrowia, Lipomyces, Cryptococcus, or Dekkera sp. cell.

Paragraph [46]. The method of any one of paragraphs [1]-[45], wherein the fermenting organism is a I. orientalis, C. lambica, S. bulderi or a S. cerevisiae cell.

Paragraph [47]. The method of any one of paragraphs [1]-[46], wherein the fermenting organism is a Saccharomyces cerevisiae cell.

Paragraph [48]. A yeast cell comprising a genetic modification that increases or decreases expression of a transporter, or regulator thereof.

Paragraph [49]. The yeast cell of paragraph [48], wherein the cell has increased or decreased expression (e.g., by at least 5%, e.g., at least 10%, at least 15%, at least 20%, at least 25%, at least 50%, at least 100%, at least 150%, at least 200%, at least 300%, or at 500%) of a transporter, or regulator thereof when compared to Saccharomyces cerevisiae strain Ethanol Red® (deposited under Accession No. V14/007039 at National Measurement Institute, Victoria, Australia) under the same conditions.

Paragraph [50]. The yeast cell of paragraph [48] or [49], wheren the cell is capable of maintaining the same yield of a fermentation product with less supplemental nitrogen (e.g., urea, ammonia, ammonium hydroxide) during fermentation, when compared to an otherwise identical fermenting organism lacking the genetic modification that increases or decreases expression of a transporter, or regulator thereof.

Paragraph [51]. The yeast cell of any one of paragraphs [48]-[50], wherein the cell comprises a genetic modification that increases or decreases expression of a transporter, such as the any one of the transporters shown in Table 1 (e.g., any one of SEQ ID NOs: 86-170).

Paragraph [52]. The yeast cell of any one of paragraphs [48]-[51], wherein the cell comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters shown in Table 1 (e.g., any one of SEQ ID NOs: 86-170).

Paragraph [53]. The yeast cell of any one of paragraphs [48]-[52], wherein the cell comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 163 or SEQ ID NO: 164.

Paragraph [54]. The yeast cell of any one of paragraphs [48]-[53], wherein the cell comprises a heterologous polynucleotide encoding a transporter that differs by no more than ten amino acids, e.g., by no more than five amino acids, by no more than four amino acids, by no more than three amino acids, by no more than two amino acids, or by one amino acid from any one of the transporters shown in Table 1 (e.g., any one of SEQ ID NOs: 86-170).

Paragraph [55]. The yeast cell of any one of paragraphs [48]-[54], wherein the cell comprises a heterologous polynucleotide encoding a transporter comprising or consisting of the amino acid sequence of any one of the transporters shown in Table 1 (e.g., any one of SEQ ID NOs: 86-170).

Paragraph [56]. The yeast cell of any one of paragraphs [48]-[55], wherein the cell comprises a disruption to an endogenous transporter gene, such as any one of the transporter genes shown in Table 1 (e.g., any one of SEQ ID NOs: 1-85).

Paragraph [57]. The yeast cell of paragraph [56], wherein the disrupted endogenous transporter gene is inactivated.

Paragraph [58]. The yeast cell of paragraph [57], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [59]. The yeast cell of paragraph [57] or [58], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [60]. The yeast cell of any one of paragraphs [48]-[59], wherein the cell comprises a genetic modification that increases or decreases expression of a regulator, such as any one of the regulators shown in Table 2 (e.g., any one of SEQ ID NOs: 231-290).

Paragraph [61]. The yeast cell of any one of paragraphs [48]-[60], wherein the cell comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [62]. The yeast cell of any one of paragraphs [48]-[61], wherein the cell comprises a heterologous polynucleotide encoding regulator, wherein the regulator differs by no more than ten amino acids, e.g., by no more than five amino acids, by no more than four amino acids, by no more than three amino acids, by no more than two amino acids, or by one amino acid from any one of the regulators shown in Table 2 (e.g., any one of SEQ ID NOs: 231-290).

Paragraph [63]. The yeast cell of any one of paragraphs [48]-[62], wherein the cell comprises a heterologous polynucleotide encoding a regulator, wherein the regulator has an amino acid sequence comprising or consisting of the amino acid sequence of any one of the regulators shown in Table 2 (e.g., any one of SEQ ID NOs: 231-290).

Paragraph [64]. The yeast cell of any one of paragraphs [48]-[63], wherein the cell comprises a disruption to an endogenous regulator gene, wheren the regulator gene is any one of the regulator genes shown in Table 2 (e.g., any one of SEQ ID NOs: 171-230).

Paragraph [65]. The yeast cell of paragraph [64], wherein the disrupted endogenous regulator gene is inactivated.

Paragraph [66]. The yeast cell of paragraph [64] or [65], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [67]. The yeast cell of any one of paragraphs [64]-[66], wherein the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [68]. The yeast cell of any one of paragraphs [48]-[67], wherein the cell comprises a heterologous polynucleotide encoding a glucoamylase.

Paragraph [69]. The yeast cell of paragraph [68], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase, such as SEQ ID NO: 102 or 103 of WO2018/222990).

Paragraph [70]. The yeast cell of any one of paragraphs [48]-[69], wherein the cell comprises a heterologous polynucleotide encoding an alpha-amylase.

Paragraph [71]. The yeast cell of paragraph [70], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [72]. The yeast cell of any one of paragraphs [48]-[71], wherein the cell comprises a heterologous polynucleotide encoding protease.

Paragraph [73]. The yeast cell of paragraph [72], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having the sequence of any one of SEQ ID NOs: 9-73 of WO2018/222990).

Paragraph [74]. The yeast cell of any one of paragraphs [48]-[73], wherein the cell is a Saccharomyces, Rhodotorula, Schizosaccharomyces, Kluyveromyces, Pichia, Issatchenkia, Hansenula, Rhodosporidium, Candida, Torulaspora, Zygosaccharomyces, Yarrowia, Lipomyces, Cryptococcus, or Dekkera sp. cell.

Paragraph [75]. The yeast cell of any one of paragraphs [48]-[74], wherein the cell is a I. orientalis, C. lambica, S. bulderi or a S. cerevisiae cell.

Paragraph [76]. The yeast cell of any one of paragraphs [48]-[75], wherein the cell is a Saccharomyces cerevisiae cell.

Paragraph [77]. A Saccharomyces cerevisiae yeast cell comprising:

(1) a heterologous polynucleotide encoding a transporter, and
(2) a heterologous polynucleotide encoding a glucoamylase, alpha-amylase, or protease;

wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164.

Paragraph [78]. The yeast cell of paragraph [77], wheren the cell comprises a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 163; and a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 164.

Paragraph [79]. The yeast cell of paragraph [77], wherein the transporter differs by no more than ten amino acids, e.g., by no more than five amino acids, by no more than four amino acids, by no more than three amino acids, by no more than two amino acids, or by one amino acid from amino acid sequence of SEQ ID NO: 163 or SEQ ID NO: 164.

Paragraph [80]. The yeast cell of paragraph [77], wherein the transporter comprises or consists of the amino acid sequence of SEQ ID NO: 163 or SEQ ID NO: 164.

Paragraph [81]. The yeast cell of any one of paragraphs [77]-[80], wherein the heterologous polynucleotide encoding the transporter is introduced into the cell using recombinant techniques.

Paragraph [82]. The yeast cell of any one of paragraphs [77]-[81], wherein the heterologous polynucleotide encoding the transporter is operably linked to a promoter that is foreign to the polynucleotide.

Paragraph [83]. The yeast cell of any one of paragraphs [77]-[80], wherein the heterologous polynucleotide encoding the transporter is introduced into the cell using non-recombinant breeding techniques.

Paragraph [84]. The yeast cell of any one of paragraphs [77]-[83], wheren the cell is capable of maintaining the same yield of a fermentation product with less supplemental nitrogen (e.g., urea, ammonia, ammonium hydroxide) during fermentation, when compared to an otherwise identical fermenting organism lacking the heterologous polynucleotide encoding the transporter.

Paragraph [85]. The yeast cell of any one of paragraphs [77]-[84], wheren the cell is capable of increased consumption of tripeptides or tetrapeptides under conditions described herein (e.g., decreased residual tripeptides or tetrapeptides in the fermentation medium after 29 hours of fermentation), when compared to an otherwise identical fermenting organism lacking the heterologous polynucleotide encoding the transporter.

Paragraph [86]. The yeast cell of any one of paragraphs [77]-[85], wherein the cell comprises a heterologous polynucleotide encoding a glucoamylase.

Paragraph [87]. The yeast cell of paragraph [86], wherein the heterologous polynucleotide encoding the glucoamylase is operably linked to a promoter that is foreign to the polynucleotide.

Paragraph [88]. The yeast cell of paragraph [86] or [87], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase, such as SEQ ID NO: 102 or 103 of WO2018/222990).

Paragraph [89]. The yeast cell of any one of paragraphs [77]-[88], wherein the cell comprises a heterologous polynucleotide encoding an alpha-amylase.

Paragraph [90]. The yeast cell of paragraph [89], wherein the heterologous polynucleotide encoding the alpha-amylase is operably linked to a promoter that is foreign to the polynucleotide.

Paragraph [91]. The yeast cell of paragraph [89] or [90], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [92]. The yeast cell of any one of paragraphs [77]-[91], wherein the cell comprises a heterologous polynucleotide encoding protease.

Paragraph [93]. The yeast cell of paragraph [92], wherein the heterologous polynucleotide encoding the protease is operably linked to a promoter that is foreign to the polynucleotide.

Paragraph [94]. The yeast cell of [92] or [93], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having the sequence of any one of SEQ ID NOs: 9-73 of WO2018/222990).

Paragraph [95]. The yeast cell of any one of paragraphs [77]-[94], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters of SEQ ID NOs: 86-162 and 165-170.

Paragraph [96]. The yeast cell of any one of paragraphs [77]-[95], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 130 or SEQ ID NO: 161.

Paragraph [97]. The yeast cell of any one of paragraphs [77]-[96], wherein the cell comprises a disruption to an endogenous transporter gene.

Paragraph [98]. The yeast cell of [97], wherein the disrupted endogenous transporter gene is inactivated.

Paragraph [99]. The yeast cell of [97] or [98], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [100]. The yeast cell of any one of paragraphs [97]-[99], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [101]. The yeast cell of any one of paragraphs [77]-[100], wherein the cell further comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [102]. The yeast cell of any one of paragraphs [77]-[101], wherein the cell comprises a disruption to an endogenous regulator gene.

Paragraph [103]. The yeast cell of [102], wherein the disrupted endogenous regulator gene is inactivated.

Paragraph [104]. The yeast cell of [102] or [103], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [105]. The yeast cell of any one of paragraphs [102]-[104], wherein the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [106]. The yeast cell of paragraph any one of paragraphs [77]-[105], wherein the cell is a recombinant cell.

Paragraph [107]. A Saccharomyces cerevisiae yeast cell comprising a heterologous polynucleotide encoding a transporter,

wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164;

and with the proviso that the yeast cell is other than:

Saccharomyces cerevisiae MBG4851 (deposited under Accession No. V14/004037 at National Measurement Institute, Victoria, Australia) or a derivative thereof,

Saccharomyces cerevisiae MBG4911 (deposited under Accession No. V15/001459 at National Measurement Institute, Victoria, Australia) or a derivative thereof,

Saccharomyces cerevisiae MBG4913 (deposited under Accession No. V15/001460 at National Measurement Institute, Victoria, Australia) or a derivative thereof,

Saccharomyces cerevisiae MBG4914 (deposited under Accession No. V15/001461 at National Measurement Institute, Victoria, Australia) or a derivative thereof,

Saccharomyces cerevisiae MBG4930 (deposited under Accession No. V15/004035 at National Measurement Institute, Victoria, Australia) or a derivative thereof,

Saccharomyces cerevisiae MBG4931 (deposited under Accession No. V15/004036 at National Measurement Institute, Victoria, Australia) or a derivative thereof,

Saccharomyces cerevisiae MBG4932 (deposited under Accession No. V15/004037 at National Measurement Institute, Victoria, Australia) or a derivative thereof.

Paragraph [108]. The yeast cell of paragraph [107], wheren the cell comprises a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 163; and a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 164.

Paragraph [109]. The yeast cell of paragraph [107], wherein the transporter differs by no more than ten amino acids, e.g., by no more than five amino acids, by no more than four amino acids, by no more than three amino acids, by no more than two amino acids, or by one amino acid from amino acid sequence of SEQ ID NO: 163 or SEQ ID NO: 164.

Paragraph [110]. The yeast cell of paragraph [107], wherein the transporter comprises or consists of the amino acid sequence of SEQ ID NO: 163 or SEQ ID NO: 164.

Paragraph [111]. The yeast cell of any one of paragraphs [107]-[110], wherein the heterologous polynucleotide encoding the transporter is introduced into the cell using recombinant techniques.

Paragraph [112]. The yeast cell of any one of paragraphs [107]-[111], wherein the heterologous polynucleotide encoding the transporter is operably linked to a promoter that is foreign to the polynucleotide.

Paragraph [113]. The yeast cell of any one of paragraphs [107]-[110], wherein the heterologous polynucleotide encoding the transporter is introduced into the cell using non-recombinant breeding techniques.

Paragraph [114]. The yeast cell of any one of paragraphs [107]-[113], wheren the cell is capable of maintaining the same yield of a fermentation product with less supplemental nitrogen (e.g., urea, ammonia, ammonium hydroxide) during fermentation, when compared to an otherwise identical fermenting organism lacking the heterologous polynucleotide encoding the transporter.

Paragraph [115]. The yeast cell of any one of paragraphs [107]-[114], wheren the cell is capable of increased consumption of tripeptides or tetrapeptides under conditions described herein (e.g., decreased residual tripeptides or tetrapeptides in the fermentation medium after 29 hours of fermentation), when compared to an otherwise identical fermenting organism lacking the heterologous polynucleotide encoding the transporter.

Paragraph [116]. The yeast cell of paragraph any one of paragraphs [107]-[115], wherein the cell further comprises a heterologous polynucleotide encoding a glucoamylase.

Paragraph [117]. The yeast cell of paragraph [116], wherein the heterologous polynucleotide encoding the glucoamylase is operably linked to a promoter that is foreign to the polynucleotide.

Paragraph [118]. The yeast cell of paragraph [116] or [117], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase, such as SEQ ID NO: 102 or 103 of WO2018/222990).

Paragraph [119]. The yeast cell of any one of paragraphs [107]-[118], wherein the cell further comprises a heterologous polynucleotide encoding an alpha-amylase.

Paragraph [120]. The yeast cell of paragraph [119], wherein the heterologous polynucleotide encoding the alpha-amylase is operably linked to a promoter that is foreign to the polynucleotide.

Paragraph [121]. The yeast cell of paragraph [119] or [120], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [122]. The yeast cell of paragraph any one of paragraphs [107]-[121], wherein the cell further comprises a heterologous polynucleotide encoding protease.

Paragraph [123]. The yeast cell of paragraph [122], wherein the heterologous polynucleotide encoding the protease is operably linked to a promoter that is foreign to the polynucleotide.

Paragraph [124]. The yeast cell of [122] or [123], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having the sequence of any one of SEQ ID NOs: 9-73 of WO2018/222990).

Paragraph [125]. The yeast cell of any one of paragraphs [107]-[124], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters of SEQ ID NOs: 86-162 and 165-170.

Paragraph [126]. The yeast cell of any one of paragraphs [107]-[125], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 130 or SEQ ID NO: 161.

Paragraph [127]. The yeast cell of any one of paragraphs [107]-[126], wherein the cell comprises a disruption to an endogenous transporter gene.

Paragraph [128]. The yeast cell of [127], wherein the disrupted endogenous transporter gene is inactivated.

Paragraph [129]. The yeast cell of [127] or [128], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [130]. The yeast cell of any one of paragraphs [127]-[129], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [131]. The yeast cell of any one of paragraphs [107]-[130], wherein the cell further comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [132]. The yeast cell of any one of paragraphs [107]-[131], wherein the cell comprises a disruption to an endogenous regulator gene.

Paragraph [133]. The yeast cell of [132], wherein the disrupted endogenous regulator gene is inactivated.

Paragraph [134]. The yeast cell of [132] or [133], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [135]. The yeast cell of any one of paragraphs [132]-[134], wherein the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [136]. The yeast cell of paragraph any one of paragraphs [107]-[135], wherein the cell is a recombinant cell.

Paragraph [137]. The yeast cell of paragraph any one of paragraphs [107]-[135], wherein the cell is non-recombinant cell.

Paragraph [138]. A yeast cell comprising a heterologous polynucleotide encoding a transporter, wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164, and wherein the yeast cell comprises a recombinant genetic modification that increases expression of the transporter.

Paragraph [139]. The yeast cell of paragraph [138], wheren the cell comprises a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 163; and a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 164.

Paragraph [140]. The yeast cell of paragraph [138], wherein the transporter differs by no more than ten amino acids, e.g., by no more than five amino acids, by no more than four amino acids, by no more than three amino acids, by no more than two amino acids, or by one amino acid from amino acid sequence of SEQ ID NO: 163 or SEQ ID NO: 164.

Paragraph [141]. The yeast cell of paragraph [138], wherein the transporter comprises or consists of the amino acid sequence of SEQ ID NO: 163 or SEQ ID NO: 164.

Paragraph [142]. The yeast cell of any one of paragraphs [138]-[141], wherein the heterologous polynucleotide encoding the transporter is operably linked to a promoter that is foreign to the polynucleotide.

Paragraph [143]. The yeast cell of any one of paragraphs [138]-[142], wherein the cell comprises multiple copies of the heterologous polynucleotide encoding the transporter.

Paragraph [144]. The yeast cell of paragraph [143], wherein coding sequences of the multiple copies are identical.

Paragraph [145]. The yeast cell of paragraph [143], wherein coding sequences of the multiple copies are non-identical.

Paragraph [146]. The yeast cell of any one of paragraphs [138]-[145], wheren the cell is capable of maintaining the same yield of a fermentation product with less supplemental nitrogen (e.g., urea, ammonia, ammonium hydroxide) during fermentation, when compared to an otherwise identical fermenting organism lacking the heterologous polynucleotide encoding the transporter.

Paragraph [147]. The yeast cell of any one of paragraphs [38]-[146], wheren the cell is capable of increased consumption of tripeptides or tetrapeptides under conditions described herein (e.g., decreased residual tripeptides or tetrapeptides in the fermentation medium after 29 hours of fermentation), when compared to an otherwise identical fermenting organism lacking the heterologous polynucleotide encoding the transporter.

Paragraph [148]. The yeast cell of any one of paragraphs [138]-[147], wherein the cell further comprises a heterologous polynucleotide encoding a glucoamylase.

Paragraph [149]. The yeast cell of paragraph [148], wherein the heterologous polynucleotide encoding the glucoamylase is operably linked to a promoter that is foreign to the polynucleotide.

Paragraph [150]. The yeast cell of paragraph [148] or [149], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase, such as SEQ ID NO: 102 or 103 of WO2018/222990).

Paragraph [151]. The yeast cell of any one of paragraphs [138]-[150], wherein the cell further comprises a heterologous polynucleotide encoding an alpha-amylase.

Paragraph [152]. The yeast cell of paragraph [151], wherein the heterologous polynucleotide encoding the alpha-amylase is operably linked to a promoter that is foreign to the polynucleotide.

Paragraph [153]. The yeast cell of paragraph [151] or [152], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [154]. The yeast cell of any one of paragraphs [138]-[153], wherein the cell further comprises a heterologous polynucleotide encoding protease.

Paragraph [155]. The yeast cell of paragraph [154], wherein the cell further comprises a heterologous polynucleotide encoding protease.

Paragraph [156]. The yeast cell of [154] or [155], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having the sequence of any one of SEQ ID NOs: 9-73 of WO2018/222990).

Paragraph [157]. The yeast cell of any one of paragraphs [138]-[156], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters of SEQ ID NOs: 86-162 and 165-170.

Paragraph [158]. The yeast cell of any one of paragraphs [138]-[157], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 130 or SEQ ID NO: 161.

Paragraph [159]. The yeast cell of any one of paragraphs [138]-[158], wherein the cell comprises a disruption to an endogenous transporter gene.

Paragraph [160]. The yeast cell of [159], wherein the disrupted endogenous transporter gene is inactivated.

Paragraph [161]. The yeast cell of [159] or [160], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [162]. The yeast cell of any one of paragraphs [159]-[161], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [163]. The yeast cell of any one of paragraphs [138]-[162], wherein the cell further comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [164]. The yeast cell of any one of paragraphs [138]-[163], wherein the cell comprises a disruption to an endogenous regulator gene.

Paragraph [165]. The yeast cell of [164], wherein the disrupted endogenous regulator gene is inactivated.

Paragraph [166]. The yeast cell of [164] or [165], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [167]. The yeast cell of any one of paragraphs [164]-[166], wherein the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [168]. The yeast cell of any one of paragraphs [138]-[167], wherein the cell is a Saccharomyces, Rhodotorula, Schizosaccharomyces, Kluyveromyces, Pichia, Issatchenkia, Hansenula, Rhodosporidium, Candida, Torulaspora, Zygosaccharomyces, Yarrowia, Lipomyces, Cryptococcus, or Dekkera sp. cell.

Paragraph [169]. The yeast cell of [168], wherein the cell is a I. orientalis, C. lambica, S. bulderi or a S. cerevisiae cell.

Paragraph [170]. The yeast cell of [169], wherein the cell is a Saccharomyces cerevisiae cell.

Paragraph [171]. A yeast cell comprising a heterologous polynucleotide encoding a transporter, wherein the transporter has at least 60%, e.g., at least 65%, 70%, 75%, 80%, 85%, 90%, 95%, 97%, 98%, 99%, or 100% sequence identity to the amino acid sequence of SEQ ID NO: 163 or SEQ ID NO: 164, and wherein the yeast further comprises a disruption to an endogenous transporter gene.

Paragraph [172]. The yeast cell of paragraph [171], wheren the cell comprises a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 163; and a heterologous polynucleotide encoding a transporter that has at least 60%, e.g., at least 65%, 70%, 75%, 80%, 85%, 90%, 95%, 97%, 98%, 99%, or 100% sequence identity to the amino acid sequence of SEQ ID NO: 164.

Paragraph [173]. The yeast cell of paragraph [171], wherein the transporter differs by no more than ten amino acids, e.g., by no more than five amino acids, by no more than four amino acids, by no more than three amino acids, by no more than two amino acids, or by one amino acid from amino acid sequence of SEQ ID NO: 163 or SEQ ID NO: 164.

Paragraph [174]. The yeast cell of paragraph [171], wherein the transporter comprises or consists of the amino acid sequence of SEQ ID NO: 163 or SEQ ID NO: 164.

Paragraph [175]. The yeast cell of any one of paragraphs [171]-[174], wherein the heterologous polynucleotide encoding the transporter is introduced into the cell using recombinant techniques.

Paragraph [176]. The yeast cell of any one of paragraphs [171]-[175], wherein the heterologous polynucleotide encoding the transporter is operably linked to a promoter that is foreign to the polynucleotide.

Paragraph [177]. The yeast cell of paragraph any one of paragraphs [171]-[174], wherein the heterologous polynucleotide encoding the transporter is introduced into the cell using non- recombinant breeding techniques.

Paragraph [178]. The yeast cell of any one of paragraphs [171]-[177], wherein the disrupted endogenous transporter gene is inactivated.

Paragraph [179]. The yeast cell of any one of paragraphs [171]-[178], wherein the coding sequence of the endogenous transporter gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the transporters shown in Table 1 (e.g., any one of SEQ ID NOs: 1-85).

Paragraph [180]. The yeast cell of any one of paragraphs [171]-[179], wherein the endogenous transporter gene encodes a transporter having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the transporters shown in Table 1 (e.g., any one of SEQ ID NOs: 86-170).

Paragraph [181]. The yeast cell of paragraph any one of paragraphs [171]-[180], wheren the cell is capable of maintaining the same yield of a fermentation product with less supplemental nitrogen (e.g., urea, ammonia, ammonium hydroxide) during fermentation, when compared to an otherwise identical fermenting organism lacking the heterologous polynucleotide encoding the transporter.

Paragraph [182]. The yeast cell of paragraph any one of paragraphs [171]-[181], wheren the cell is capable of increased consumption of tripeptides or tetrapeptides under conditions described herein (e.g., decreased residual tripeptides or tetrapeptides in the fermentation medium after 29 hours of fermentation), when compared to an otherwise identical fermenting organism lacking the heterologous polynucleotide encoding the transporter.

Paragraph [183]. The yeast cell of paragraph any one of paragraphs [171]-[182], wherein the cell further comprises a heterologous polynucleotide encoding a glucoamylase.

Paragraph [184]. The yeast cell of paragraph [183], wherein the heterologous polynucleotide encoding the glucoamylase is operably linked to a promoter that is foreign to the polynucleotide.

Paragraph [185]. The yeast cell of paragraph [183] or [184], wherein the glucoamylase is a Pycnoporus glycoamylase (e.g. a Pycnoporus sanguineus glucoamylase described herein), a Gloeophyllum glucoamylase (e.g. a Gloeophyllum sepiarium or Gloeophyllum trabeum glucoamylase described herein), or a Saccharomycopsis glucoamylase (e.g., a Saccharomycopsis fibuligera glucoamylase, such as SEQ ID NO: 102 or 103 of WO2018/222990).

Paragraph [186]. The yeast cell of any one of paragraphs [171]-[185], wherein the cell further comprises a heterologous polynucleotide encoding an alpha-amylase.

Paragraph [187]. The yeast cell of paragraph [186], wherein the heterologous polynucleotide encoding the alpha-amylase is operably linked to a promoter that is foreign to the polynucleotide.

Paragraph [188]. The yeast cell of paragraph [186] or [187], wherein the alpha-amylase is a Bacillus alpha-amylase (e.g., a Bacillus stearothermophilus, Bacillus amyloliquefaciens, or Bacillus licheniformis alpha-amylase described herein), or a Debaryomyces alpha-amylase (e.g., a Debaryomyces occidentalis alpha-amylase described herein).

Paragraph [189]. The yeast cell of any one of paragraphs [171]-[188], wherein the cell further comprises a heterologous polynucleotide encoding protease.

Paragraph [190]. The yeast cell of paragraph [189], wherein the heterologous polynucleotide encoding the protease is operably linked to a promoter that is foreign to the polynucleotide.

Paragraph [191]. The yeast cell of paragraph [189] or [190], wherein the protease is a Meripilus giganteus, Trametes versicolor, Dichomitus squalens, Polyporus arcularius, Lenzites betulinus, Ganoderma lucidum, Neolentinus lepideus, or Bacillus sp. 19138 protease (e.g., a protease having the sequence of any one of SEQ ID NOs: 9-73 of WO2018/222990).

Paragraph [192]. The yeast cell of any one of paragraphs [171]-[191], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of any one of the transporters of SEQ ID NOs: 86-162 and 165-170.

Paragraph [193]. The yeast cell of any one of paragraphs [171]-[192], wherein the cell further comprises a heterologous polynucleotide encoding a transporter having at least 60%, e.g., at least 65%, 70%, 75%, 80%, 85%, 90%, 95%, 97%, 98%, 99%, or 100% sequence identity to the amino acid sequence of SEQ ID NO: 129, SEQ ID NO: 130 or SEQ ID NO: 161.

Paragraph [194]. The yeast cell of any one of paragraphs [171]-[193], wherein the cell further comprises a heterologous polynucleotide encoding a regulator, wherein the regulator has at least 60%, e.g., at least 65%, 70%, 75%, 80%, 85%, 90%, 95%, 97%, 98%, 99%, or 100% sequence identity to the amino acid sequence of any one of the regulators shown in Table 2 (e.g., any one of SEQ ID NOs: 231-290).

Paragraph [195]. The yeast cell of any one of paragraphs [171]-[194], wherein the cell comprises a disruption to an endogenous regulator gene.

Paragraph [196]. The yeast cell of [195], wherein the disrupted endogenous regulator gene is inactivated.

Paragraph [197]. The yeast cell of [195] or [196], wherein the coding sequence of the endogenous regulator gene has at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the regulator genes shown in Table 2 (e.g., any one of SEQ ID NOs: 171-230).

Paragraph [198]. The yeast cell of any one of paragraphs [195]-[197], wherein the endogenous gene encodes a regulator having at least 60%, e.g., at least 65%, at least 70%, at least 75%, at least 80%, at least 85%, at least 90%, at least 95%, at least 96%, at least 97%, at least 98%, at least 99%, or 100% sequence identity to the any one of the regulators shown in Table 2 (e.g., any one of SEQ ID NOs: 231-290).

Paragraph [199]. The yeast cell of any one of paragraphs [171]-[199], wherein the cell is a Saccharomyces, Rhodotorula, Schizosaccharomyces, Kluyveromyces, Pichia, Issatchenkia, Hansenula, Rhodosporidium, Candida, Torulaspora, Zygosaccharomyces, Yarrowia, Lipomyces, Cryptococcus, or Dekkera sp. cell.

Paragraph [200]. The yeast cell of [199], wherein the cell is a I. orientalis, C. lambica, S. bulderi or a S. cerevisiae cell.

Paragraph [201]. The yeast cell of [200], wherein the cell is a Saccharomyces cerevisiae cell.

Paragraph [202]. A composition comprising the yeast strain of any one of paragraphs [77]-[201]and one or more naturally occurring and/or non-naturally occurring components, such as components are selected from the group consisting of: surfactants, emulsifiers, gums, swelling agents, and antioxidants.

Paragraph [203]. A method of producing a derivative of a yeast strain of paragraph [77]-[201], the method comprising:
(a) providing:
   (i) a first yeast strain; and
   (ii) a second yeast strain, wherein the second yeast strain is a strain of any one of paragraphs [77]-[201];
(b) culturing the first yeast strain and the second yeast strain under conditions which permit combining of DNA between the first and second yeast strains;
(c) screening or selecting for a derivatived yeast strain comprising the heterologous polynucleotide encoding the transporter.

Paragraph [204]. A method of producing ethanol, comprising incubating a strain of any one of paragraphs [77]-[201] with a substrate comprising a fermentable sugar under conditions which permit fermentation of the fermentable sugar to produce ethanol.

Paragraph [205]. Use of a strain of any one of paragraphs [77]-[201] in the production of ethanol.

Paragraph [206]. A method of producing a fermentation product from a starch-containing or cellulosic-containing material comprising:

(a) saccharifying the starch-containing or cellulosic-containing material; and
(b) fermenting the saccharified material of step (a) with the yeast cell of any one of paragraphs [77]-[201].

Paragraph [207]. The method of paragraph [206], comprising liquefying the starch-containing material at a temperature above the initial gelatinization temperature in the presence of an alpha-amylase prior to saccharification.

Paragraph [208]. The method of paragraph [207], comprising adding a protease in liquefaction.

Paragraph [209]. The method of paragraph [208], wherein the protease is a serine protease, e.g., an S8 protease.

Paragraph [210]. The method of paragraph [208] or [209], wherein the protease is a bacterial protease, particularly a protease derived form Pyrococcus, Palaeococcus, or Thermococcus, more particularly Pyrococcus furiosus, Palaeococcus ferrophilus, Thermococcus litoralis, Thermococcus thioreducens.

Paragraph [211]. The method of any one of paragraphs [208]-[210], wherein the protease is selected from the group consisting of SEQ ID NO: 291, SEQ ID NO: 292, SEQ ID NO: 293, SEQ ID NO: 294, and SEQ ID NO: 295, or a variant of any one of SEQ ID NO: 291, SEQ ID NO: 292, SEQ ID NO: 293, SEQ ID NO: 294, and SEQ ID NO: 295 having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity thereto.

Paragraph [212]. The method of any one of paragraphs [206]-[211], wherein fermentation and saccharification are performed simultaneously in a simultaneous saccharification and fermentation (SSF).

Paragraph [213]. The method of any one of paragraphs [206]-[211], wherein fermentation and saccharification are performed sequentially (SHF).

Paragraph [214]. The method of any one of paragraphs [206]-[213], comprising recovering the fermentation product from the from the fermentation.

Paragraph [215]. The method of paragraph [214], wherein recovering the fermentation product from the from the fermentation comprises distillation.

Paragraph [216]. The method of any one of paragraphs [206]-[215], wherein the fermentation product is ethanol.

Paragraph [217]. The method of paragraph [216], wherein the ethanol yield is more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, greater than Saccharomyces cerevisiae strain Ethanol Red® (deposited under Accession No. V14/007039 at National Measurement Institute, Victoria, Australia) under the same conditions (e.g., under conditions described herein, such as after 53 hours fermentation).

Paragraph [218]. The method of paragraph [216] or [217], wherein the ethanol yield is more than 1.0%, e.g., more than 2.0%, more than 2.5%, more than 3.0%, more than 3.5%, more than 4.0%, more than 4.5%, more than 5.0%, more than 5.5%, more than 6.0%, more than 6.5%, more than 7.0%, more than 7.5%, more than 8.0%, more than 8.5%, more than 9.0%, more than 9.5%, or more than 10.0%, greater when compared to an otherwise identical fermenting organism lacking the heterologous polynucleotide encoding the transporter.

Paragraph [219]. The method of any one of paragraphs [206]-[218], wherein saccharification of step (a) occurs on a starch-containing material, and wherein the starch-containing material is either gelatinized or ungelatinized starch.

Paragraph [220]. The method of any one of paragraphs [206]-[219], wherein saccharification of step (a) occurs on a cellulosic-containing material, and wherein the cellulosic-containing material is pretreated.

Paragraph [221]. The method of paragraph [220], wherein the pretreatment is a dilute acid pretreatment.

Paragraph [222]. The method of any one of paragraphs [206]-[221], wherein saccharification occurs on a cellulosic-containing material, and wherein the enzyme composition comprises one or more enzymes selected from a cellulase, an AA9 polypeptide, a hemicellulase, a CIP, an esterase, an expansin, a ligninolytic enzyme, an oxidoreductase, a pectinase, a protease, and a swollenin.

Paragraph [223]. The method of paragraph [222], wherein the cellulase is one or more enzymes selected from an endoglucanase, a cellobiohydrolase, and a beta-glucosidase.

Paragraph [224]. The method of paragraph [222], wherein the hemicellulase is one or more enzymes selected a xylanase, an acetylxylan esterase, a feruloyl esterase, an arabinofuranosidase, a xylosidase, and a glucuronidase.

### Deposit of Biological Material

The following biological material has been deposited under the terms of the Budapest Treaty with the National Measurement Institute, Victoria, Australia and given the following accession number:

| Deposit | Accession Number | Date of Deposit |
|---|---|---|
| Ethanol Red ® | V14/007039 | March 19, 2014 |
| MBG4851 | V14/004037 | Feb. 17, 2014 |
| MBG4911 | V15/001459 | Jan. 13, 2015 |
| MBG4913 | V15/001460 | Jan. 13, 2015 |
| MBG4914 | V15/001461 | Jan. 13, 2015 |
| MBG4930 | V15/004035 | Feb. 19, 2015 |
| MBG4931 | V15/004036 | Feb. 19, 2015 |
| MBG4932 | V15/004037 | Feb. 19, 2015 |

The strains have been deposited under conditions that assure that access to the culture will be available during the pendency of this patent application to one determined by the Commissioner of Patents and Trademarks to be entitled thereto under 37 C.F.R. § 1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. All references are specifically incorporated by reference for that which is described.

The following examples are offered to illustrate certain aspects of the present invention, but not in any way intended to limit the scope of the invention as claimed.

### EXAMPLES

### Example 1: Peptide Uptake

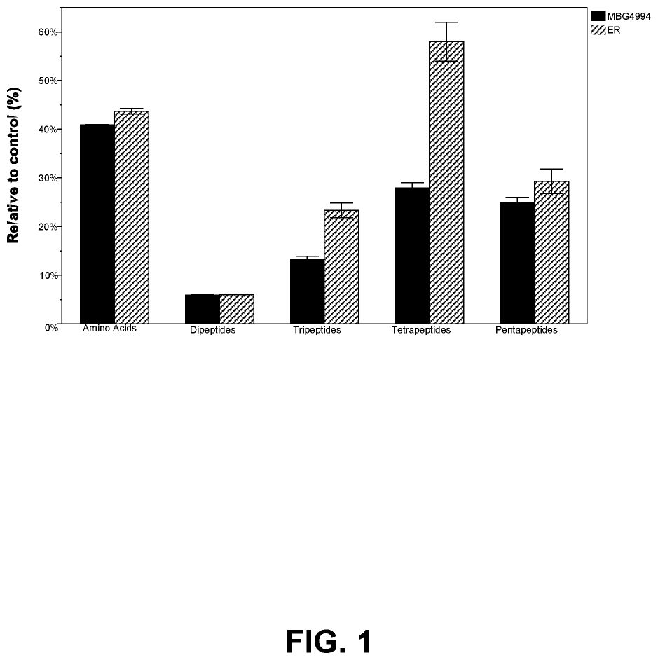

Peptide uptake for strains MBG4994 (which comprises the expression cassates for FotX and Fot2 genes; and was prepared according the breeding procedures described in U.S. Pat. No. 8,257,959) vs Ethanol Red® (Fermentis/Lesaffre, USA) was determined at 24 h (relative to time 0 h) during corn mash ethanol fermentation. Corn mash was industrially generated at an ethanol plant using Avantec® Amp (commercially available enzyme from Novozymes A/S containing an alpha-amylase and a protease). Fermentation was carried out in 125 ml flasks containing 50 grams of corn mash inoculated with 10 million cells/g, and dosed with a cocktail of glucoamylase enzymes. No urea was added to the corn mash. Tubes were incubated at 32° C. FIG. 1 shows significant improvement in uptake of tripeptide and tetrapeptides by MBG4994 compared to Ethanol Red®.

### Example 2: Deletion of a Transporter Genes from a Yeast Strain

This example describes the construction of a yeast strain containing a deletion of a gene encoding a transporter or other gene involved in nitrogen metabolism. First, a protospacer sequence was added to a CRISPR/Cas9 base vector to direct a double-strand break to the locus of interest. The plasmid created by the protospacer addition was then transformed into yeast along with repair DNA. This repair DNA is homologous to the region 5′ of the start codon of the gene of interest at the 5′ end of the repair DNA and is homologous to the region 3′ of the stop codon of the gene of interest at the 3′ end of the repair DNA. Thus, the Cas9 protein as encoded in the plasmid vector cut the desired DNA based on homology to the protospacer sequence at the locus of interest, and then the repair DNA repaired the cut with DNA lacking the coding region of the gene of interest, resulting in deletion of the gene of interest.

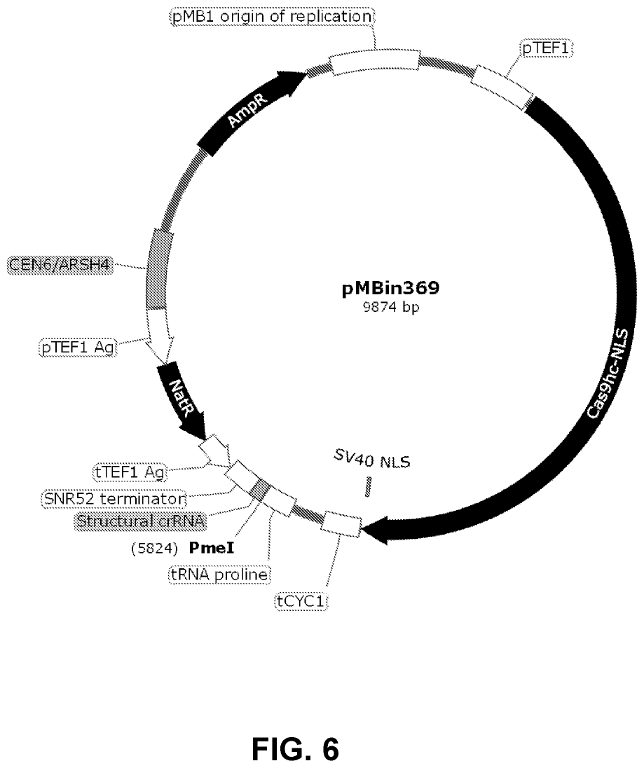

The CRISPR/Cas9 base vector used was pMBin369 (See FIG. 6): a yeast episomal plasmid containing a nourseothricin resistance marker for selection in yeast, an expression cassette for Cas9-NLS, and a Pmel restriction site between the tRNA promoter and the structural crRNA encoding sequence. To target a gene for cutting by Cas9, a protospacer adjacent to an NGG sequence was found for each gene of interest. A linear oligonucleotide was obtained containing the DNA sequence homologous to 5′ of the Pmel site in pMBin369 (5-ATTCCCAGCTCGCCCC-3′; SEQ ID NO: 296), the protospacer sequence as shown in Table 3, and the DNA sequence homologous to 3′ of the Pmel site in pMBin369 (5-GTTTTAGAGCTAGAAA-3′; SEQ ID NO: 297). The linear, single-stranded oligonucleotide was then added to Pmel digested pMBin369 using the NEBuilder® HiFi DNA Assembly Master Mix (New England BioLabs) per the manufacturer's instructions.

To target deletion of each gene of interest, repair oligonucleotides were designed to remove the entire open reading frame of the gene of interest. One of the repair oligonucleotides was in the forward direction of the gene of interest and contained the 45 bases just 5′ of the gene of interest, ending at the nucleotide just prior to ATG start codon of the gene of interest, fused directly to the 45 bases just 3′ of the gene of interest, starting at the nucleotide immediately 3′ of stop codon of the gene of interest. The other repair oligonucleotide was the reverse complement of the forward repair oligonucleotide. Sequences of the repair oligonucleotides used can be found in Table 4. Prior to use in yeast transformation, the two oligonucleotides for a gene of interest were annealed into a double-strand repair DNA via mixing of the two oligonucleotides together, heating the mixture to 98° C., and then slowly cooling to room temperature to allow oligonucleotide annealing.

To create the desired yeast strains with a gene of interest deleted, the CRISPR/Cas9 plasmid and the appropriate repair DNAs as shown in Table 3 were transformed into the yeast strain of interest following a yeast electroporation protocol (See, Thompson et al. Yeast. 1998 Apr. 30;14(6):565-71). Transformants were selected on YPD+clonNAT to select for transformants that contain the CRISPR/Cas9 plasmid. Individual transformant colonies were picked to a new YPD+cloNAT plate and then screened for deletion of the gene of interest using PCR with locus specific primers.

TABLE 3

| Gene Name | Plasmid name | Protospacer sequence | Repair oligos |
|---|---|---|---|
| Fot2 | pMHCT415 | 5′-ATGTAGAAAAAGGAGAACAA-3′ | 1226919 + 1226927 |
| | | (SEQ ID NO: 298) | |
| FotX | pMHCT417 | 5′-AGAAAGAAAAAGGATCTCAA-3′ | 1226920 + 1226928 |
| | | (SEQ ID NO: 299) | |
| opt1 | pMHCT419 | 5′-GGAGAATTGGGCAAGTATGT-3′ | 1226921 + 1226929 |
| | | (SEQ ID NO: 300) | |
| 0pt2 | pMHCT421 | 5′-GATGAGAAGGTATCCACAAA-3′ | 1226922 + 1226930 |
| | | (SEQ ID NO: 301) | |
| YGL114W | pMHCT428 | 5′-ACACTCCGAGCAACCATAGC-3′ | 1226925 + 1226933 |
| | | (SEQ ID NO: 302) | |
| ptr1 | pMHCT425 | 5′-TCCGTTGCTGATGATGATTT-3′ | 1226924 + 1226932 |
| | | (SEQ ID NO: 303) | |
| pt12 | pMHCT424 | 5′-GTCTTCTCCTCTTCGATGAC-3′ | 1226923 + 1226931 |
| | | (SEQ ID NO: 304) | |
| dal5 | pMHCT414 | 5′-TGGTGTCACTTCGATTTCTT-3′ | 1226918 + 1226926 |
| | | (SEQ ID NO: 305) | |

TABLE 4

| Repair | |
|---|---|
| oligo | |
| name | Repair oligo sequence |
| 1226918 | 5′-ACAACAAAAC AAGGATAATC AAATAGTGTA AAAAAAAAAT TCAAGAATAG TGGTGAGATA |
| | CACTGACATT ATTGTTATCA GAGAAGAAT A-3′ (SEQ ID NO: 306) |
| 1226919 | 5′-CAGTCTATTC CATTTGACTG AAGAAAAAAA AATTTATTAC TGAAGAATAG TCGCCACAA |
| | AGAGGATGCA AACCAATGGT GCGAAACACT G-3′ (SEQ ID NO: 307) |
| 1226920 | 5′-TACGACGTTC CATTCGACTG AAAAAAAAAA TATTTATCGC TGAAGGGCAG TTGCCATCAC |
| | AGGACGACGG AAATCCCATG GTGAGGGACT-3′ (SEQ ID NO: 308) |
| 1226921 | 5′-TGTGGCAGAA AATAACCGCA ACAATTATAT AACGTCACAG AACACTCATG AAAACATAAT |
| | AATAAACCTG CAGAGGTTCA TATCAAGTTT-3′ (SEQ ID NO: 309) |
| 1226922 | 5′-GTAGGGTTTG TTATACGCAA TATTGCTGTT GAATTATTAG AAATTCTAGA ACTTTTCTTA GAACGCTACC |
| | ATTTCTTTAT TTTTTTTGCA-3′ (SEQ ID NO: 310) |
| 1226923 | 5′-TTTATTTTTT TTTCTTTTGA ATTAGATCAC TAATAAACTC TTATATGCGT TTAATTAACT TACTGTCTTT |
| | TTTTTTTTTT TTTTAACCAT-3′ (SEQ ID NO: 311) |
| 1226924 | 5′-TCCCTAATCT TTACAGGTCA CACAAATTAC ATAGAACATT CCAATATTCA AGCCCTCTAC TATGTTTTAT |
| | AGTTGACATA TTTGTATATA-3′ (SEQ ID NO: 312) |
| 1226925 | 5′-GACTAACCGT TAAAGATTCT AAATCGGTAC TGTAAATACT TTGAAGGTAT ATAATATTTT |
| | ACTAATAAAG AATTTACAAA GTTTACAAAT-3′ (SEQ ID NO: 313) |
| 1226926 | 5′-TATTCTTCTC TGATAACAAT AATGTCAGTG TATCTCACCA CTATTCTTGA ATTTTTTTTT TACACTATTT |
| | GATTATCCTTG TTTTGTTGT-3′ (SEQ ID NO: 314) |
| 1226927 | 5′-CAGTGTTTCG CACCATTGGT TTGCATCCTC TTTGTGGCGA CTATTCTTCA GTAATAAATT TTTTTTTCTT |
| | CAGTCAAATG GAATAGACTG-3′ (SEQ ID NO: 315) |
| 1226928 | 5′-AGTCCCTCAC CATGGGATTT CCGTCGTCCT GTGATGGCAA CTGCCCTTCA GCGATAAATA TTTTTTTTTT |
| | CAGTCGAATG GAACGTCGTA-3′ (SEQ ID NO: 316) |
| 1226929 | 5′-AAACTTGATA TGAACCTCTG CAGGTTTATT ATTATGTTTT CATGAGTGTT CTGTGACGTT ATATAATTGT |
| | TGCGGTTATT TTCTGCCACA-3′ (SEQ ID NO: 317) |
| 1226930 | 5′-TGCAAAAAAA ATAAAGAAAT GGTAGCGTTC TAAGAAAAGT TCTAGAATTT CTAATAATTC |
| | AACAGCAATA TTGCGTATAA CAAACCCTAC-3′ (SEQ ID NO: 318) |
| 1226931 | 5′-ATGGTTAAAA AAAAAAAAAA AAAGACAGTA AGTTAATTAA ACG CATATAA GAGTTTATTA |
| | GTGATCTAAT TCAAAAGAAA AAAAAAATAA-3′ (SEQ ID NO: 319) |
| 1226932 | 5′-TATATACAAA TATGTCAACT ATAAAACATA GTAGAGGGCT TGAATATTGG AATGTTCTAT |
| | GTAATTTGTG TGACCTGTAA AGATTAGGGA-3′ (SEQ ID NO: 320) |
| 1226933 | 5′-ATTTGTAAAC TTTGTAAATT CTTTATTAGTA AAATATTATA TACCTTCAAA GTATTTACAG TACCGATTTA |
| | GAATCTTTAA CGGTTAGTC-3′ (SEQ ID NO: 321) |

### Example 3: Deletion of Additional Gene(s) from a Yeast Strain Already Containing One or More Deleted Genes

This example describes the deletion of an additional gene encoding a transporter or other gene involved in nitrogen metabolism in a yeast strain that contains an exisiting deletion of a gene encoding a transporter or other gene involved in nitrogen metabolism.

To remove the CRISPR/Cas9 plasmid from a deletion strain as constructed in Example 2, the yeast strain of interest was streaked for single colonies on a YPD plate. Once colonies formed, a few were picked and simultaneously patched to a YPD plate and to a YPD+clonNAT plate. An isolated colony that grew on YPD but failed to grow on YPD+clonNAT was chosen, since lack of growth on YPD+clonNAT indicated that the CRISPR/Cas9 plasmid had been lost.

The deletion strain just described was then transformed with a new CRISPR/Cas9 plasmid and annealed repair oligonucleotides, and a correctly deleted isolate was isolated as described in Example 2.

This process was used iteratively; for instance, a strain containing three deletions was transformed to delete a gene as described in Example 2 three times, with two intervening plasmid removal steps.

### Example 4: Recombinant Addition of Transporter Genes to Saccharomyces cerevisiae Yeast

The following example describes the construction of yeast strains containing an expression cassette for either Fot2 or FotX, or for an expression cassette containing both the Fot2 and FotX genes. The Fot genes were PCR amplified from yeast strain MBG4994 (either individually or as a pair) and then integrated into the X-3 locus (as described in Mikkelsen at al 2012) of the Saccharomyces cerevisiae yeast strain Ethanol Red® or integrated into the X-3 locus of an Ethanol Red® derived strain with opt1Δ opt2Δ ygl114wΔ.

To amplify the promoter, coding region, and terminator for each Fot gene from MBG4994, PCR primers were designed to amplify from approximately 1,000 bases 5′ of the gene of interest's start codon to approximately 500 bases 3′ of the gene of interest's stop codon. Flanking DNA for the X-3 locus was added to the 5′ end of each oligonucleotide to allow targeting of the amplicon to the X-3 integration site of Ethanol Red®. The resulting primer sequences are shown in Table 3. To make the Fot2 integration DNA, a PCR was performed using MBG4994 genomic DNA, primers 1229007 and 1229008 (Table 5), and Taq DNA polymerase (New England BioLabs) per the manufacturer's instructions. To make the FotX integration DNA, a PCR was performed using MBG4994 genomic DNA, primers 1229009 and 1229010 (Table 5), and Taq DNA polymerase (New England BioLabs) per the manufacturer's instructions. Since Fot2 and FotX are adjacent to one another on the MBG4994 genome, a single PCR can be used to make an amplicon containing both genes: To make the Fot2+FotX integration DNA, a PCR was performed using MBG4994 genomic DNA, primers 1229007 and 1229010 (Table 5), and Taq DNA polymerase (New England BioLabs) per the manufacturer's instructions.

To create the desired Ethanol Red® derived yeast strains with an ectopic Fot expression cassette, the yeast strain Ethanol Red® was transformed following a yeast electroporation protocol with one of the Fot containing amplicons described above and pMCTS442, a CRISPR/Cas9 plasmid for yeast with a plasmid containing Cas9-NLS, guide RNA specific to X-3, and the nourseothricin selection marker. Transformants were selected on YPD+clonNAT to select for transformants that contain the CRISPR/Cas9 plasmid. Individual transformant colonies were picked to a new YPD+clonNAT plate and then screened for integration at X-3 using PCR with X-3 locus specific primers.

To create the desired Ethanol Red® opt1Δ opt2Δ ygl114wΔ derived yeast strains with an ectopic Fot expression cassette, the yeast strain Ethanol Red® opt1Δ opt2Δ ygl114wΔ was transformed and transformants screened as described for Ethanol Red® strain supra.

TABLE 5

| Primer | Primer Sequence |
|---|---|
| 1229007 | 5′-GAAACAATAG GCAAGAAGTA GGCGAGAGCC |
| | GACATACGAG ACTAATGTGT CCGCACTGAG |
| | GCGGAAGCAT TTGAGCCATC-3′ (SEQ ID NO: 322) |
| 1229008 | 5′-TCTTGAGCTC GTCCTTTTAC TAGCATATCA |
| | ATATCCGTTT CATTGAAAAG TGGTTTGCAT |
| | CAACACAGGA TGAACAGCAT GAATGTGCC-3′ |
| | (SEQ ID NO: 323) |
| 1229009 | 5′-GAAACAATAG GCAAGAAGTA GGCGAGAGCC |
| | GACATACGAG ACTAATGTGT CCGCCATGGA |
| | AGGATGTTTT GATAAGACCG GATGACG-3′ |
| | (SEQ ID NO: 324) |
| 1229010 | 5′-CGCTCTTGAG CTCGTCCTTT TACTAGCATA |
| | TCAATATCCG TTTCATTGAA AAGTGGGCCC |
| | TTCATTATTC TTCTCAAGTC TCTGACGCG-3′ |
| | (SEQ ID NO: 325) |
| 1218018 | 5′-GTTACTGTTG TCCACAGGC-3′ (SEQ ID NO: 326) |
| 1218019 | 5′-CTTGCTGCAT GGAGACAAGT G-3′ (SEQ ID |
| | NO: 327) |

To determine if the Fot2, FotX or Fot2 and FotX insertion in the yeast genome contained the DNA sequence as expected, PCR was used to amplify the expression cassette integrated at the X-3 locus. The primers used were 1218018 and 1218019 (Table 5). The resulting PCR amplification product was DNA sequenced. The transformants differed from Fot2 (SEQ ID NO: 163) and FotX (SEQ ID NO: 164) as shown in Table 6.

TABLE 6
Protein sequence information for yeast strains with Fot2, FotX, or Fot2 and FotX expression
cassettes at the X-3 locus. The pre-fix “het_” in front of a change indicates that the change was
heterozygous (change occurred on one of the two copies of the expression cassette in the isolated
diploid transformants). Mutations without the “het_” pre-fix are homozygous.

| | | Expressed | |
|---|---|---|---|
| Strain | Background | Gene | Mutation |
| 1 | Ethanol Red ® | Fot2 | S2P, K39E, H232L, I324V |
| 2 | Ethanol Red ® | Fot2 | Al1V, E22V, N481D |
| 3 | Ethanol Red ® | Fot2 | I128N, N208D, K311E |
| 4 | Ethanol Red ® | FotX | F491S |
| 5 | Ethanol Red ® | FotX | V41I, L413L, T442S, W445C |
| 6 | Ethanol Red ® + | Fot2 | T462I |
| | ΔOPT1ΔOPT2ΔYGL114 | | |
| 7 | Ethanol Red ® + | Fot2 | Q73L, R524T |
| | ΔOPT1ΔOPT2ΔYGL114 | | |
| 8 | Ethanol Red ® + | FotX | I113T, A527V |
| | ΔOPT1ΔOPT2ΔYGL114 | | |
| 9 | Ethanol Red ® + | FotX | (no changes) |
| | ΔOPT1ΔOPT2ΔYGL114 | | |
| 10 | Ethanol Red ® + | FotX | N397K, het_F158L, het_I314T, het_M329L |
| | ΔOPT1ΔOPT2ΔYGL114 | | |
| 11 | Ethanol Red ® + | FotX | T442A |
| | ΔOPT1ΔOPT2ΔYGL114 | | |
| 12 | Ethanol Red ® + | Fot2 and | Fot2 changes: L149P, A472V |
| | ΔOPT1ΔOPT2ΔYGL114 | FotX | FotX changes: K354STOP, A471V |
| 13 | Ethanol Red ® + | Fot2 and | Fot2 changes: Y94N, N417D |
| | ΔOPT1ΔOPT2ΔYGL114 | FotX | FotX changes: K25R, L128M, L162S, Q217R, |
| | | | W333R,Y370STOP |

### Example 5: Impact of Deletion of Peptide Transporters on Ethanol and Peptide Uptake During Ethanol Fermentation Using Corn Mash Produced Industrially by a Liquefaction Blend

This example describes the evaluation of yeast strains containing a deletion of one or more genes encoding a transporter or transporters involved in amino nitrogen uptake and metabolism. Particularly, the impact on ethanol kinetics, final ethanol titer and the uptake of peptides during ethanol fermentation with an industrially prepared corn mash is compared among the yeast strains listed in Table 7.

TABLE 7
Strains used in fermentation
4994 (MBG4994)
4994ΔFOT2ΔFOTX
4994ΔOPT1ΔOPT2Δygl114w
4994ΔOPT1ΔOPT2Δygl114wΔFOT2ΔFOTX
ER (Ethanol Reda); Fermentis/Lesaffre, USA)
ERΔOPT1ΔOPT2Δygl114w

### Seed Culture

Cryo-preserved cultures of strains were first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flasks were incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water. The resuspended cells were used to inoculate the corn mash at the beginning of simultaneous saccharification and fermentation (SSF).

### Corn Mash

Industrially prepared corn mash liquefied with Avantec® Amp (commercially available enzyme from Novozymes A/S containing an alpha-amylase and a protease) was obtained from an ethanol plant. The mash contained 34.5% dried solids as measured by Mettler-Toledo HB43-S moisture balance. The mash was supplemented with 3 ppm of antibiotic LACTROL™and its pH was adjusted to 5.0 prior to use in SSF.

### Simultaneous Saccharification and Fermentation (SSF)

All fermentations were carried out in 125-ml baffled flasks with screw caps having a 0.5 mm hole. Flasks were filled with 40-50 g corn mash and inoculated with resuspended seed culture at 10 million cells per gram of mash. A commercially available glucoamylase enzyme blend (Innova® Excel L) was added to flasks at 0.06% (w/w) of dry corn solids. Fermentation was run for 52 hr, during which samples were taken periodically to analyze the residual peptides and ethanol in the fermented corn mash.

### Peptide Analysis

Samples (5 g) removed from flasks during fermentation were transferred into 15 ml conical tubes containing 50 μl of 40% v/v H₂SO₄, vortexed, and centrifuged at 3,500 rpm for 10 min at 22° C. The resulting supernatant was filtered through a 0.2 μm syringe filter. Filtered samples were stored at −20° C. prior to preparation for LCMS analysis. Peptides were derivatized using the AccQTag™ Ultra Derivatization Kit (Waters Inc) according to the following procedure: Mix 10 μl sample with 70 μl AccQ TAG Ultra Borate buffer and 20 μl AccQ TAG Ultra reagent in a microcentrifuge tube. Wait 1 minute and then add 10 μl 100 mM DTT-solution and mix. Incubate for 20 minutes at 60 degrees celcius. Cool samples. Add 5 μl iodoacetamide (500 mM). Wait 30 min for samples to cool in darkness. Derivatized samples are subsequently analyzed on a reverse phase LCMS orbitrap (Thermo Qexactive) in positive ionization scanning mode. Samples are subsequent analyzed on a Accela LC-system equipped with a reverse phase column (ACQUITY UPLC CSH C18 Column, 130 Å, 1.7 μm, 2.1 mm×150 mm) coupled to a Q Exactive Hybrid Quadrupole-Orbitrap Mass spectrometer (Thermo Scientific). The mass spectrometer was set in positive ionization scanning mode from 200 to 1500 m/z. The LC-system was setup with two mobile phases: MQ-water with 0.1% formic acid (A-eluent) and acetonitrile with 0.1% formic acid (B-eluent). The LC-flow was set to 0.25 ml/min running a standard gradient from 99% A-eluent to 65% A-eluent in 25 minutes.

All peaks are annotated (with retention time and exact mass) and integrated in a LCMS peak picking software (GeneData). Identification of peptides is based matching between observed peaks and a) a peptide database with theoretic peptides or b) a database with possible hydrolysis products of a known substrate.

### Ethanol Analysis

Samples (5 g) removed from flasks during fermentation were transferred into 15 ml conical tubes containing 50 μl of 40% v/v H₂SO₄, vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

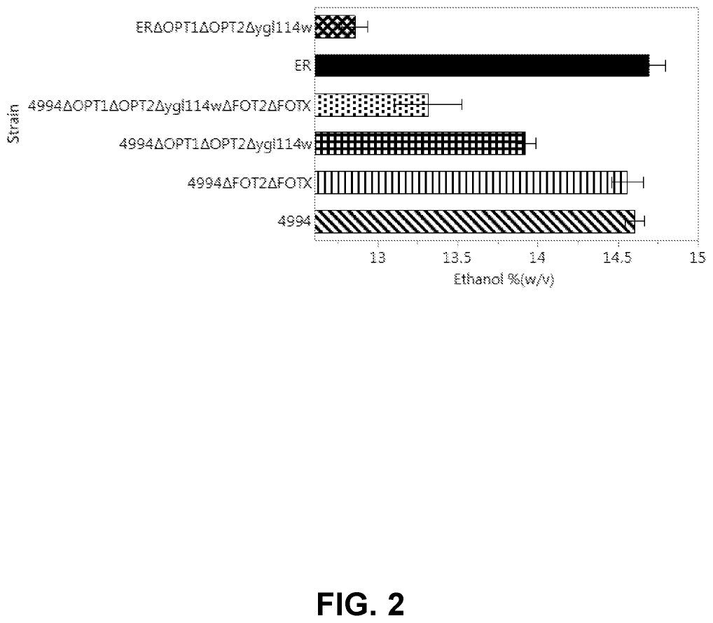

FIG. 2 shows the final ethanol titers of corn mash fermentation by the mutant strains and their corresponding parent strains as listed in Table 7. Deletion of FOTX and FOT2 gens in MBG4994 does not impact fermentation performance of 4994ΔFOT2ΔFOTX compared to MBG4994, likely due to the compensating activity of other oligopeptide transporters encoded by OPT1, OPT2 and WGL114w. The triple deletion of genes encoding for oligopeptide transporters (OPT1, OPT2 and YGL114w) has a greater negative impact on ethanol fermentation kinetics and final titer of Ethanol Red® than the corresponding knockout strain of MBG4994. As shown in FIG. 2, the final ethanol titer with 4994ΔOPT1ΔOPT2Δygl114w strain is higher than the ERΔOPT1ΔOPT2Δygl114w. The difference in kinetics and final ethanol titer between the latter two strains is attributed to the presence of FOTX and FOT2 genes in MBG4994 and 4994ΔOPT1ΔOPT2Δygl114w. The additional deletion of FOTX and FOT2 in 4994ΔOPT1ΔOPT2Δygl114w strains further slows down fermentation kinetics and reduces the final ethanol titer to a level closer to that of ERΔOPT1ΔOPT2Δygl114w strain, suggesting that FOT2 and FOTX improve fermentation performance by enabling yeast to access and uptake more amino nitrogen during corn mash fermentation.

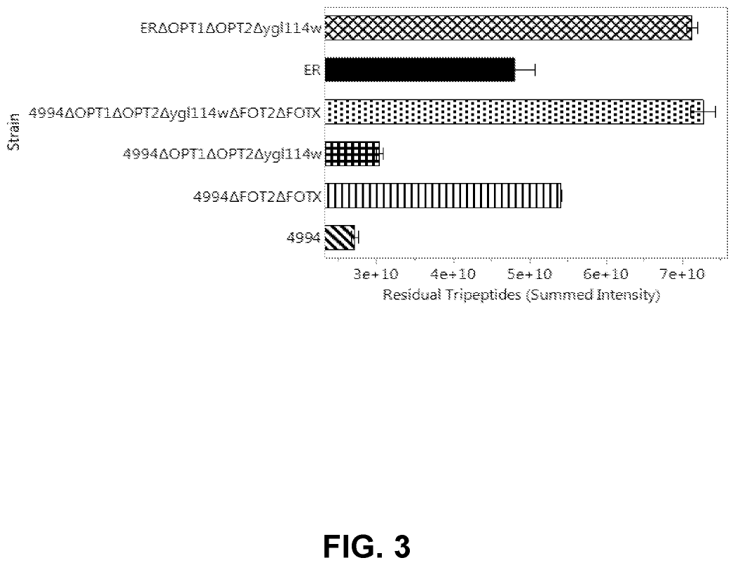

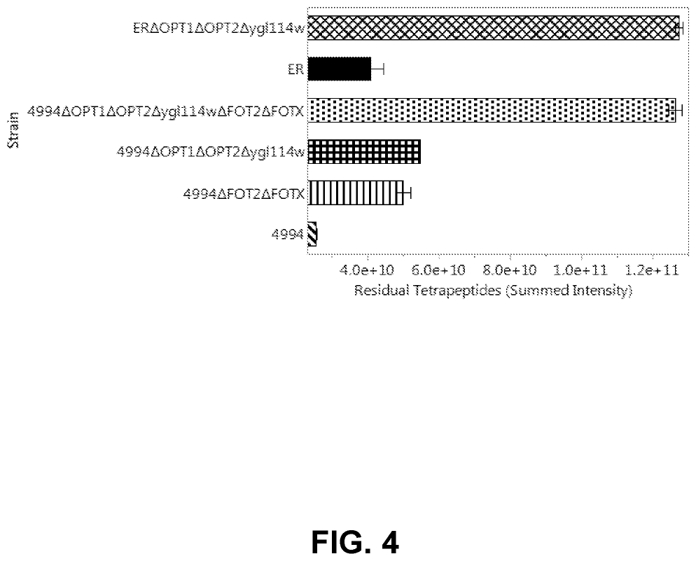

FIGS. 3 and 4 show the residual tripeptide and residual tetrapeptide, respectively, after 29 h fermentation using industrially prepared corn mash with the strains listed in Table 7. After 29 h fermentation, Ethanol Red® has greater residual tri- and tetrapeptides when compared to MBG4994, indicating that MBG4994 can uptake more tri- and tetrapeptides during corn mash fermentation. Deletion of FOTX and FOT2 in MBG4994 leads to increased residual tri- and tetrapeptides similar to the residual tri- and tetrapeptides levels with Ethanol Red®. Uptake of tri- and tetrapeptides by MBG4994 is less impacted than Ethanol Red® when OPT1, OPT2 and ygl114w genes are deleted, which may be attributed to compensating peptide uptake activities from the Fot2 and Fotx transporters. Additional deletion of the FOT2 and FOTX genes to the MBG4994-triple knockout strain (4994ΔOPT1ΔOPT2Δygl114w) resulted in the yeast strain 4994ΔOPT1ΔOPT2Δygl114wΔFOT2ΔFOTX that exhibits a phenotype (i.e. inability to uptake tri- and tetrapeptides) similar to that of Ethanol Red® triple deletion strain (ERΔOPT1ΔOPT2Δygl114w), suggesting that FOT2 and FOTX genes improve the uptake of tri- and tetrapeptides and provide MBG4994 access to an expanded range of peptides that leads to improved fermentation performance.

### Example 6: Impact of Expression of Fundal Oligopeptide Peptide Transporters (FOTX and FOT2) in Recombinant Yeast Strains on Ethanol Fermentation

This example describes the evaluation of recombinant yeast strains containing one or more heterologous genes encoding a fungal oligopeptide transporter or transporters encoded by FOTX or FOT2 which are involved in amino nitrogen uptake and metabolism. Particularly, the impact on ethanol final ethanol titer during ethanol fermentation with an industrially prepared corn mash is compared among the yeast strains listed in Table 8.

TABLE 8

| Strains | Description |
|---|---|
| MBG4994 | Parent strain |
| ER | Ethanol Red ® (Fermentis/Lesaffre, USA) |
| ER::FOT2 A | Ethanol Red ® expressing FOT2 with |
| | S2P, K39E, H232L, I324V |
| ER::FOT2 B | Ethanol Red ® expressing FOT2 with |
| | A11V, E22V, N481D |
| ER::FOT2 C | Ethanol Red ® expressing FOT2 with |
| | I128N, N208D, K311E |
| ER::FOTX A | Ethanol Red ® expressing FOTX with |
| | V41I, L413L, T442S, W445C |

### Seed Culture

Cryo-preserved cultures of strains were first grown in liquid YPD media (Yeast extract, 10 g; Peptone, 20 g; Dextrose, 60 g; dissolved in 1 L of distilled water). Cultivation was done aseptically in a sterile 125-ml Erlenmeyer flask filled with 50 ml YPD media and inoculated with 100 μl of cryo-preserved culture. Flasks were incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water. The resuspended cells were used to inoculate the corn mash at the beginning of simultaneous saccharification and fermentation (SSF).

### Corn Mash

Industrially prepared corn mash liquefied with Avantec® Amp (commercially available enzyme from Novozymes A/S containing an alpha-amylase and a protease) was obtained from an ethanol plant. The mash contained 34.5% dried solids as measured by Mettler-Toledo HB43-S moisture balance. The mash was supplemented with 3 ppm of antibiotic LACTROL™ and its pH was adjusted to 5.0 prior to use in SSF.

### Simultaneous Saccharification and Fermentation (SSF)

Fermentations were carried out in 125-ml baffled flasks with screw caps having a 0.5 mm hole. Flasks were filled with 40-50 g corn mash and inoculated with resuspended seed culture at 10 million cells per gram of mash. A commercially available glucoamylase enzyme blend (Innova® Excel L) was added to flasks at 0.06% (w/w) of dry corn solids. Fermentation was run for 53 hr, during which samples were taken periodically to analyze ethanol in the fermented corn mash.

### Ethanol Analysis

Samples (5 g) removed from flasks during fermentation were transferred into 15 ml conical tubes containing 50 μL of 40% v/v H₂SO₄, vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

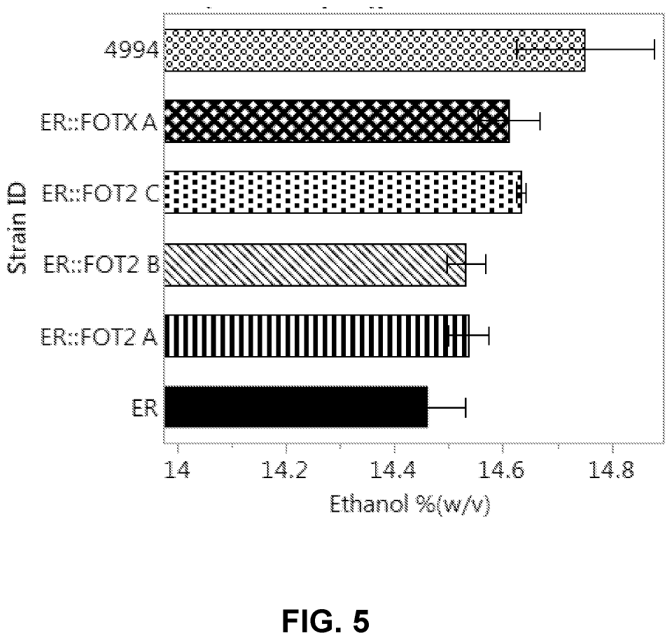

FIG. 5 shows the final ethanol titers after 53 h fermentation of industrially prepared corn mash by the mutant strains and their corresponding parent strains as listed in Table 8. MBG4994 produced higher ethanol titer than Ethanol Red®, and expressing FOTX or FOT2 in Ethanol Red® leads to higher ethanol titer compared to the Ethanol Red® parent strain, demonstrating expression of FOTX or FOT2 improves the performance of yeast during corn mash ethanol fermentation.

### Example 7: Efficient Utilization of Amino Nitrogen by Yeast Enables Significant Urea Reduction in Corn Mash Fermentations Without Impacting Ethanol Yield

This example describes the evaluation of yeast strains MBG4994 (which expresses FOTX and FOT2) compared to Ethanol Red® (which lacks expression of FOTX and FOT2) in corn mash fermentations prepared without any proteases with varied exogenous nitrogen (i.e. urea) concentrations. Particularly, the impact of limited nitrogen availability on final ethanol titer in a corn mash fermentation is studied.

### Seed Culture

Cryo-preserved cultures of strains were first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flasks were incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water. The resuspended cells were used to inoculate the corn mash at the beginning of simultaneous saccharification and fermentation (SSF).

### Corn Mash

Industrially prepared corn mash samples liquefied with either Avantec® Amp (commercially available liquefaction enzyme containing an alpha-amylase and a protease) or Liquozyme® LpH (commercially available liquefaction enzyme containing an alpha-amylase and no protease) were obtained from ethanol plants. The mash samples contained 34-35% dried solids as measured by Mettler-Toledo HB43-S moisture balance. The mash was supplemented with 2 ppm of antibiotic LACTROL™ and its pH was adjusted to 5.0 prior to use in SSF. The mash prepared using Liquozyme® LpH (here referred to as LpH) was augmented with 0, 200, 400, and 800 ppm urea, whereas the mash prepared with Avantec® Amp was not supplemented with any urea.

### Simultaneous Saccharification and Fermentation (SSF)

All fermentations were carried out in 15-ml flip-cap tubes with caps having a 0.5 mm hole. Tubes were filled with 4-5 g corn mash and inoculated with resuspended seed culture at 10 million cells per gram of mash. A commercially available glucoamylase enzyme blend (Spirizyme® Excel) was added to flasks at 0.04% (w/w) of dry corn solids. Fermentation was run for 68 hr. Samples were taken after 68 hours of fermentation to analyze ethanol in the fermented corn mash.

### Ethanol Analysis

To each sample, 50 μL of 40% v/v H₂SO₄ was added to the corn mash. Samples were vortexed and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

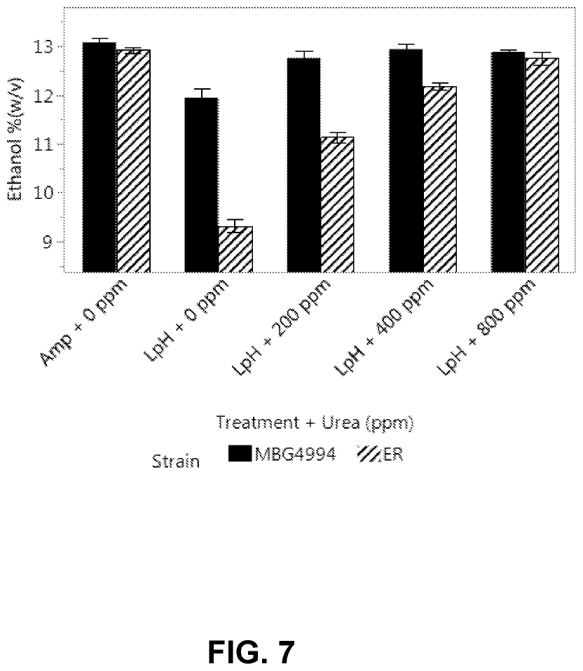

FIG. 7 illustrates the final ethanol titers of Ethanol Red® and MBG4994 after 68 h fermentation in corn mash with varied urea concentrations. Results show MBG4994 ethanol yield is not impacted as significantly as Ethanol Red® by urea reduction. MBG4994 requires up to 50% less urea compared to Ethanol Red® reach maximum ethanol titers. The higher ethanol yield at low urea concentrations compared to Ethanol Red® suggests the FOT genes in MBG4994 allow access to an expanded range of amino nitrogen.

1-25. (canceled)

26. A recombinant Saccharomyces cerevisiae yeast cell comprising:
(1) a heterologous polynucleotide encoding a transporter, and
(2) a heterologous polynucleotide encoding a glucoamylase, alpha-amylase, or protease;
wherein the transporter has at least 60% sequence identity to the amino acid sequence of SEQ ID NO: 163 or SEQ ID NO: 164.

27. The yeast cell of claim 26, wherein the heterologous polynucleotide of (2) encodes a glucoamylase.

28. The yeast cell of claim 26, wherein the heterologous polynucleotide of (2) encodes an alpha amylase.

29. The yeast cell of claim 26, wherein the heterologous polynucleotide encoding the transporter comprises the amino acid sequence of SEQ ID NO: 163 or SEQ ID NO: 164.

30. The yeast cell of claim 26, wherein the heterologous polynucleotide encoding the transporter is introduced into the cell using recombinant techniques.

31. The yeast cell of claim 26, wherein the heterologous polynucleotide encoding the transporter is operably linked to a promoter that is foreign to the polynucleotide.

32. The yeast cell of claim 26, wherein the heterologous polynucleotide encoding the glucoamylase, alpha-amylase, or protease is operably linked to a promoter that is foreign to the polynucleotide.

33. The yeast cell of claim 26, wherein the heterologous polynucleotide encoding the transporter is introduced into the cell using non-recombinant breeding techniques.

34. The yeast cell of claim 26, wherein the cell is capable of maintaining the same yield of a fermentation product with less supplemental nitrogen (e.g., urea, ammonia, ammonium hydroxide) during fermentation, when compared to an otherwise identical fermenting organism lacking the heterologous polynucleotide encoding the transporter.

35. The yeast cell of claim 26, wherein the cell is capable of decreasing residual tripeptides or tetrapeptides following fermentation, when compared to an otherwise identical yeast lacking the heterologous polynucleotide encoding the transporter.

36. The yeast cell of claim 26, wherein the cell comprises multiple copies of the heterologous polynucleotide encoding the transporter.

37. A composition comprising the yeast strain of claim 26, and one or more components selected from the group consisting of: surfactants, emulsifiers, gums, swelling agents and antioxidants.

38. A method of producing a derivative of a yeast strain of claim 26, the method comprising:
(a) providing:
(i) a first yeast strain; and
(ii) a second yeast strain, wherein the second yeast strain is yeast cell of claim 26;
(b) culturing the first yeast strain and the second yeast strain under conditions which permit combining of DNA between the first and second yeast strains;
(c) screening or selecting for a derivative yeast strain comprising the heterologous polynucleotide encoding the transporter of claim 26.

39. A method of producing ethanol, comprising incubating a yeast cell of claim 26 with a substrate comprising a fermentable sugar under conditions which permit fermentation of the fermentable sugar to produce ethanol.

40. A method of producing a fermentation product from a starch-containing or cellulosic-containing material comprising:
(a) saccharifying the starch-containing or cellulosic-containing material; and
(b) fermenting the saccharified material of step (a) with the yeast cell of claim 26.

41. The method of claim 40, comprising liquefying the starch-containing material at a temperature above the initial gelatinization temperature in the presence of an alpha-amylase prior to saccharification.

42. The method of claim 40, comprising adding a protease in liquefaction.

43. The method of claim 40, wherein fermentation and saccharification are performed simultaneously in a simultaneous saccharification and fermentation (SSF).

44. The method of claim 40, comprising recovering the fermentation product from the fermentation.

45. The method of claim 40, wherein the fermentation product is ethanol.
